(19) United States
(12) Patent Application Publication
(10) Pub. No.: US 2004/0219629 A1
(43) Pub. Date: Nov. 4, 2004

(54) Increasing carotenoid production in bacteria via chromosomal integration

(76) Inventors: Qiong Cheng, Wilmington, DE (US); Pierre E. Rouviere, Wilmington, DE (US); Wonchul Suh, Hockessin, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/735,442

(22) Filed: Dec. 12, 2003

Related U.S. Application Data
(60) Provisional application No. 60/434,618, filed on Dec. 19, 2002.

(51) Int. Cl. C12P 23/00; C12N 1/21

(52) U.S. Cl. 435/67; 435/252.33

(57) ABSTRACT

The present invention relates to carotenoid overproducing bacteria The genes of the isoprenoid pathway in the bacterial hosts of the invention have been engineered such that certain genes are either up-regulated or down regulated resulting in the production of carotenoid compounds at a higher level than is found in the un-modified host. Genes that may be up-regulated include the dxs, idi, ispB, lytB and ygbBP genes. Additionally it has been found that a partial disruption of the yjeR gene has the effect of enhancing carotenoid production.

This application claims the benefit of U.S. Provisional Application No. 60/434,618 filed Dec. 19, 2002.

### FIELD OF THE INVENTION

This invention is in the field of microbiology. More specifically, this invention pertains to carotenoid overproducing bacterial strains.

### BACKGROUND OF THE INVENTION

Carotenoids are pigments that are ubiquitous throughout nature and synthesized by all oxygen evolving photosynthetic organisms and in some heterotrophic growing bacteria and fungi. Industrial uses of carotenoids include pharmaceuticals, food supplements, electro-optic applications, animal feed additives, and colorants in cosmetics, to mention a few. Because animals are unable to synthesize carotenoids de novo, they must obtain them by dietary means. Thus, manipulation of carotenoid production and composition in plants or bacteria can provide new or improved sources of carotenoids.

Carotenoids come in many different forms and chemical structures. Most naturally occurring carotenoids are hydrophobic tetraterpenoids containing a C₄₀ methyl-branched hydrocarbon backbone derived from successive condensation of eight C₅ isoprene units (isopentenyl pyrophosphate, IPP). In addition, novel carotenoids with longer or shorter backbones occur in some species of nonphotosynthetic bacteria.

The genetics of carotenoid pigment biosynthesis are well-known (Armstrong et al., J. Bact., 176: 4795-4802 (1994); Armstrong et al., Annu. Rev. Microbiol., 51:629-659 (1997)). This pathway is extremely well-studied in the Gram-negative, pigmented bacteria of the genera Pantoea, formerly known as Erwinia. In both E. herbicola EHO-10 (ATCC 39368) and E. uredovora 20D3 (ATCC 19321), the crt genes are clustered in two operons, crtZ and crtEXYIB (U.S. Pat. No. 5,656,472; U.S. Pat. No. 5,545,816; U.S. Pat. No. 5,530,189; U.S. Pat. No. 5,530,188; and U.S. Pat. No. 5,429,939).

Isoprenoids constitute the largest class of natural products in nature, and serve as precursors for sterols (eukaryotic membrane stabilizers), gibberelinns and abscisic acid (plant hormones), menaquinone, plastoquinones, and ubiquinone (used as carriers for electron transport), tetrapyrroles as well as carotenoids and the phytol side chain of chlorophyll (pigments for photosynthesis). All isoprenoids are synthesized via a common metabolic precursor, isopentenyl pyrophosphate (IPP). Until recently, the biosynthesis of IPP was generally assumed to proceed exclusively from acetyl-CoA via the classical mevalonate pathway. However, the existence of an alternative, mevalonate-independent pathway for IPP formation has been characterized in eubacteria and green algae.

E. coli contains genes that encode enzymes of the mevalonate-independent pathway of isoprenoid biosynthesis (FIG. 1). In this pathway, isoprenoid biosynthesis starts with the condensation of pyruvate with glyceraldehyde-3-phosphate (G3P) to form deoxy-D-xylulose via the enzyme encoded by the dxs gene. A host of additional enzymes are then used in subsequent sequential reactions, converting deoxy-D-xylulose to the final C5 isoprene product, isopentenyl pyrophosphate (IPP). IPP is converted to the isomer dimethylallyl pyrophosphate (DMAPP) via the enzyme encoded by the idi gene. IPP is condensed with DMAPP to form C10 geranyl pyrophosphate (GPP) which is then elongated to C15 farnesyl pyrophosphate (FPP).

FPP synthesis is common in both carotenogenic and non-carotenogenic bacteria. E. coli does not normally contain the genes necessary for conversion of FPP to β-carotene (FIG. 1). Enzymes in the subsequent carotenoid pathway generate carotenoid pigments from the FPP precursor and can be divided into two categories: carotene backbone synthesis enzymes and subsequent modification enzymes. The backbone synthesis enzymes include geranyl geranyl pyrophosphate synthase (CrtE), phytoene synthase (CrtB), phytoene dehydrogenase (CrtI) and lycopene cyclase (CrtY/L), etc. The modification enzymes include ketolases, hydroxylases, dehydratases, glycosylases, etc.

E. coli is a convenient host for heterologous carotenoid production. Most of the carotenogenic genes from bacteria, fungi and higher plants can be functionally expressed in E. coli (Sandmann, G., Trends in Plant Science, 6:14-17 (2001)). Furthermore, many genetic tools are available for use in E. coli, a production host often used for large-scale bioprocesses.

Engineering E. coli for increased carotenoid production has previously focused on overexpression of key isoprenoid pathway genes from multi-copy plasmids. It has been postulated that the total amount of carotenoids produced in non-carotenogenic hosts is limited by the availability of terpenoid precursors (Albrecht et al., Biotechnol. Lett., 21:791-795 (1999)). Several studies have reported between a 1.5× and 50× increase in carotenoid formation in such E. coli systems upon cloning and transformation of plasmids encoding isopentenyl diphosphate isomerase (idi), deoxy-D-xylulose-5-phosphate (DXP) synthase (dxs), DXP reductoisomerase (dxr) from various sources (Kim, S., and Keasling, J., Biotech. Bioeng., 72:408415 (2001); Mathews, P., and Wurtzel, E., Appl. Microbiol. Biotechnol., 53:396-400 (2000); Harker, M., and Bramley, P., FEBS Letter., 448:115-119 (1999); Misawa, N., and Shimada, H., J. Biotechnol., 59:169-181 (1998); Liao et al., Biotechnol. Bioeng., 62:235-241 (1999); and Misawa et al., Biochem. J., 324:421-426 (1997)). In addition, it has also been reported that increasing isoprenoid precursor concentration may be lethal (Sandmann, G., supra).

The highest level of carotenoids produced to date in E. coli are around 1.57 mg/g dry cell weight (DCW). In contrast, engineered strains of Candida utilis produce 7.8 mg of lycopene per gram of dry cell weight of lycopene (Sandmann, supra). It has been speculated that the limits for carotenoid production in a non-carotenogenic host, such as E. coli, had been reached at the level of around 1.5 mg/g DCW due to carotenoid overload of the membranes, disrupting membrane functionality. Because of this, it has been suggested that the future focus of engineering E. coli for high levels of carotenoid production should be on formation of additional membranes (Albrecht et al., supra).

Most of the work to date in the metabolic engineering of isoprenoids has been done using carotenoids primarily because of the easy color screening. Engineering an increased supply of isoprenoid precursors for increased production of carotenoids is necessary. It has been shown that a rate-limiting step in carotenoid biosynthesis is the isomerization of IPP to DMAPP (Kajiwara et al., Biochem. J., 423: 421-426 (1997)). It was also found that the conversion from FPP to GGPP is the first functional limiting step for the production of carotenoids in E. coli (Wang et al., Biotchnol. Prog., 62: 235-241 (1999)). Transformation of E. coli for overexpression of the dxs, dxr, and idi genes was found to increase production of carotenoids by a factor of 3.5 (Albrecht et al., supra). To avoid competition from other pathways and to relieve the limiting steps, a GGPP synthase (gps) from Archaroglobus fulgidus was cloned in a multi-copy expression vector and over-expressed in E. coli, along with the E. coli idi gene (Wang et al., supra). These examples show that a multi-copy expression vector has been widely used for the metabolic engineering for the production of carotenoids.

The problem to be solved, therefore, is to engineer and provide microbial hosts which are capable of producing increased levels of carotenoids. Applicants have solved the stated problem by making modifications to the E. coli chromosome, increasing β-carotene production up to 6 mg per gram dry cell weight (6000 PPM), an increase of 30-fold over initial levels; with no lethal effect.

### SUMMARY OF THE INVENTION

The invention provides a carotenoid overproducing bacteria comprising the genes encoding a functional carotenoid enzymatic biosynthetic pathway wherein the dxs, idi and ygbBP genes are overexpressed and wherein the yjeR gene is down regulated.

Additionally the invention provides a carotenoid overproducing bacteria comprising the genes encoding a functional carotenoid enzymatic biosynthetic pathway wherein the dxs, idi, ygbBP and ispB genes are overexpressed. Optionally the lytB gene may also be overexpressed to further enhance the carotenoid production.

In a preferred embodiment, the invention provides a carotenoid overproducing bacteria selected from the group consisting of a strain having the ATCC identification number PTA-4807 and a strain having the ATCC identification number PTA-4823

In another embodiment the invention provides a method for the production of a carotenoid comprising:

a) growing the carotenoid overproducing bacteria of the invention the bacteria overexpressing at least one gene selected from the group consisting of dxs, idi ygbBP, ispB, lytB, dxr, wherein yjeR is optionally downregulated, for a time sufficient to produce a carotenoid; and

b) optionally recovering the carotenoid from the carotenoid overproducing bacteria of step (a).

### BRIEF DESCRIPTION OF THE DRAWINGS AND SEQUENCE DESCRIPTIONS

FIG. 1 outlines the isoprenoid and carotenoid biosynthetic pathways used for production of β-carotene in E. coli.

FIG. 2 shows the strategy for chromosomal integration of promoter or full gene sequences and stacking the strong promoter-isoprenoid gene fusions.

FIG. 3 shows PCR analysis of chromosomal insertions.

FIG. 4 shows PCR analysis of chromosomal insertions.

FIG. 5 shows PCR analysis of chromosomal insertions.

FIG. 6 shows the plasmid map of pSUH5.

FIG. 7 shows the plasmid map of pPCB15.

FIG. 8 shows the strategy for creating E. coli Tn5 mutants which have increased carotenoid production.

FIG. 9 shows increased β-carotene production from an E. coli Tn5 mutant.

FIG. 10 shows insertion site of Tn5 in the Y15; yjeR::Tn5 mutation.

FIG. 11 shows β-carotene production by the engineered E. coli strains of the present invention.

FIG. 12 shows bacteriophage P1 mediated transduction and parallel combinatorial stacking used in the optimization of β-carotene production.

The invention can be more fully understood from the following detailed description and the accompanying sequence descriptions, which form a part of this application.

The following sequences comply with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

| Gene/Protein | | Nucleotide | Amino Acid |
|---|---|---|---|
| Product | Source | SEQ ID NO | SEQ ID NO |
| CrtE | Pantoea stewartii | 1 | 2 |
| CrtX | Pantoea stewartii | 3 | 4 |
| CrtY | Pantoea stewartii | 5 | 6 |
| CrtI | Pantoea stewartii | 7 | 8 |
| CrtB | Pantoea stewartii | 9 | 10 |
| CrtZ | Pantoea stewartii | 11 | 12 |
| dxs(16a) | Methylomonas 16a | 13 | 14 |
| lytB(16a) | Methylomonas 16a | 15 | 16 |
| dxr(16a) | Methylomonas 16a | 17 | 18 |

SEQ ID NOs:19-20 are oligonucleotide primers used to amplify the carotenoid biosynthesis genes from P. stewartii.

SEQ ID NOs:21-32 are oligonucleotide primers used to create chromosomal integration of the T5 strong promoter (P_{T5}) upstream from E. coli isoprenoid genes in the present invention.

SEQ ID NO:33 is the nucleotide sequence of the P_{T5} promoter sequence inserted in pKD4 to create pSUH5.

SEQ ID NO:34-45 are oligonucleotide primers for creating dxs(16a), dxr(16a), and lytB(16a) gene insertions in the E. coli chromosome.

SEQ ID NO:46-62 are oligonucleotide primers used for screening to confirm correct insertion of chromosomal integrations in the present invention.

SEQ ID NO:63 is the nucleotide sequence of the yjeR::Tn5 mutant gene.

SEQ ID NO:64 is the nucleotide sequence for plasmid pPCB15.

SEQ ID NO:65 is the nucleotide sequence for plasmid pKD46.

SEQ ID NO:66 is the nucleotide sequence for plasmid pSUH5.

### BRIEF DESCRIPTION OF BIOLOGICAL DEPOSITS

The following biological deposit have been made under the terms of the Budapest Treaty on the International Recognition of the Deposit of Microorganisms for the purposes of Patent Procedure:

| Depositor Identification | Int'l. Depository | |
|---|---|---|
| Reference | Designation | Date of Deposit |
| Plasmid pCP20 | ATCC# PTA-4455 | Jun. 13, 2002 |
| Methylomonas 16a | ATCC# PTA-2402 | Aug. 22, 2000 |
| WS#124 E. coli strain P_{T5}-dxs | ATCC# PTA-4807 | Nov. 20, 2002 |
| P_{T5}-idi P_{T5}-ygbBP yjeR::Tn5, | | |
| pPCB15 | | |
| WS#208 E. coli strain P_{T5}-dxs | ATCC# PTA-4823 | Nov. 26, 2002 |
| P_{T5}-idi P_{T5}-ygbBP P_{T5}-ispB, | | |
| pDCQ108 | | |

As used herein, “ATCC” refers to the American Type Culture Collection International Depository Authority located at ATCC, 10801 University Blvd., Manassas, Va. 20110-2209, USA. The “International Depository Designation” is the accession number to the culture on deposit with ATCC.

The listed deposits will be maintained in the indicated international depository for at least thirty (30) years and will be made available to the public upon the grant of a patent disclosing it. The availability of a deposit does not constitute a license to practice the subject invention in derogation of patent rights granted by government action.

### DETAILED DESCRIPTION OF THE INVENTION

In this disclosure, a number of terms and abbreviations are used. The following definitions are provided.

“Open reading frame” is abbreviated ORF.

“Polymerase chain reaction” is abbreviated PCR.

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

The term “isoprenoid” or “terpenoid” refers to the compounds and any molecules derived from the isoprenoid pathway including 10 carbon terpenoids and their derivatives, such as carotenoids and xanthophylls.

A “carotene” refers to a hydrocarbon carotenoid. Carotene derivatives that contain one or more oxygen atoms, in the form of hydroxy-, methoxy-, oxo-, epoxy-, carboxy-, or aldehydic functional groups, or within glycosides, glycoside esters, or sulfates, are collectively known as “xanthophylls”. Carotenoids are furthermore described as being acyclic, monocyclic, or bicyclic depending on whether the ends of the hydrocarbon backbones have been cyclized to yield aliphatic or cyclic ring structures (G. Armstrong, (1999) In Comprehensive Natural Products Chemistry, Elsevier Press, volume 2, pp 321-352).

The terms “λ-Red recombination system”, “λ-Red system” and “λ-Red recombinase” are used interchangeably to describe a group of enzymes encoded by the bacteriophage λ genes exo, bet, and gam. The enzymes encoded by the three genes work together to increase the rate of homologous recombination in E. coli, an organism generally considered to have a relatively low rate of homologous recombination; especially when using linear integration cassettes. The λ-Red system facilitates the ability to use short regions of homology (10-50 bp) flanking linear double-stranded (ds) DNA fragments for homologous recombination. In the present method, the λ-Red genes are expressed on helper plasmid pKD46 (Datsenko and Wanner, PNAS, 97:6640-6645 (2000); SEQ ID NO:65).

The terms “Methylomonas 16a strain” and “Methylomonas 16a” are used interchangeably and refer to a bacterium (ATCC PTA-2402) of a physiological group of bacteria known as methylotrophs, which are unique in their ability to utilize methane as a sole carbon and energy source.

The term “yjeR” refers to the oligo-ribonuclease gene locus.

The term “Dxs” refers to the enzyme D-1-deoxyxylulose 5-phosphate encoded by the dxs gene which catalyzes the condensation of pyruvate and D-glyceraldehyde 3-phosphate to D-1-deoxyxylulose 5-phosphate (DOXP).

The terms “Dxr” or “IspC” refer to the enzyme DOXP reductoisomerase encoded by the dxr or ispC gene that catalyzes the simultaneous reduction and isomerization of DOXP to 2-C-methyl-D-erythritol-4-phosphate. The names of the gene, dxr or ispC, are used interchangeably in this application. The names of gene product, Dxr or IspC are used interchangeably in this application.

The term “YgbP” or “IspD” and refers to the enzyme encoded by the ygbB or ispD gene that catalyzes the CTP-dependent cytidylation of 2-C-methyl-D-erythritol-4-phosphate to 4-diphosphocytidyl-2C-methyl-D-erythritol. The names of the gene, ygbP or ispD, are used interchangeably in this application. The names of gene product, YgbP or IspD are used interchangeably in this application.

The term “YchB” or “IspE” and refers to the enzyme encoded by the ychB or ispE gene that catalyzes the ATP-dependent phosphorylation of 4-diphosphocytidyl-2C-methyl-D-erythritol to 4-diphosphocytidyl-2C-methyl-D-erythritol-2-phosphate. The names of the gene, ychB or ispE, are used interchangeably in this application. The names of gene product, YchB or IspE are used interchangeably in this application.

The term “YgbB” or “IspF” refers to the enzyme encoded by the ygbB or ispF gene that catalyzes the cyclization with loss of CMP of 4-diphosphocytidyl-2C-methyl-D-erythritol to 4-diphosphocytidyl-2C-methyl-D-erythritol-2-phosphate to 2C-methyl-D-erythritol-2,4-cyclodiphosphate. The names of the gene, ygbB or ispF, are used interchangeably in this application. The names of gene product, YgbB or IspF are used interchangeably in this application.

The term “GcpE” or “IspG” refers to the enzyme encoded by the gcpE or ispG gene that is involved in conversion of 2C-methyl-D-erythritol-2,4-cyclodiphosphate to 1-hydroxy-2-methyl-2-(E)-butenyl 4-diphosphate. The names of the gene, gcpE or ispG, are used interchangeably in this application. The names of gene product, GcpE or IspG are used interchangeably in this application.

The term “LytB” or “IspH” refers to the enzyme encoded by the lytB or ispH gene and is involved in conversion of 1-hydroxy-2-methyl-2-(E)-butenyl 4-diphosphate to isopentenyl diphosphate (IPP) and dimethylallyl diphosphate (DMAPP). The names of the gene, lytB or ispH, are used interchangeably in this application. The names of gene product, LytB or IspH are used interchangeably in this application.

The term “Idi” refers to the enzyme isopentenyl diphosphate isomerase encoded by the idi gene that converts isopentenyl diphosphate to dimethylallyl diphosphate.

The term “IspA” refers to the enzyme farnesyl pyrophosphate (FPP) synthase encoded by the ispA gene.

The term “IspB” refers to the enzyme octaprenyl diphosphate synthase, which supplies the precursor of the side chain of the isoprenoid quinones encoded by the ispB gene.

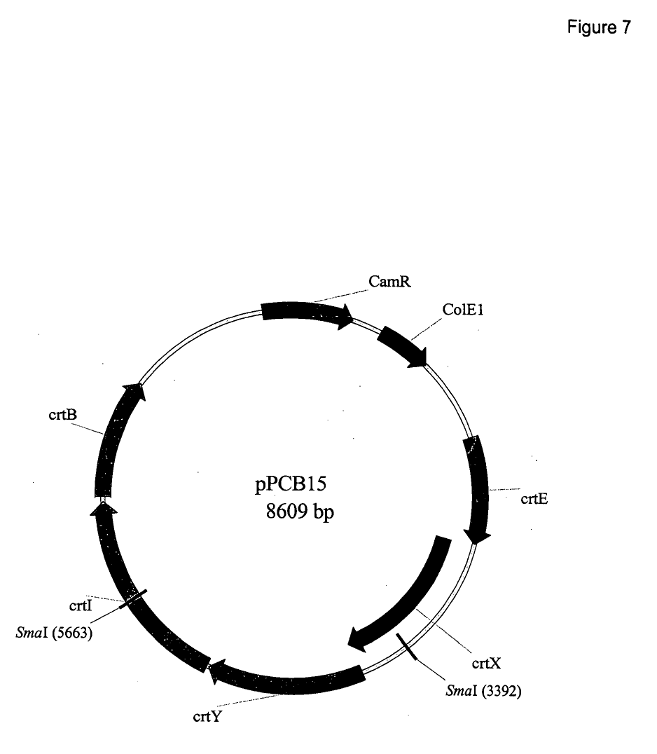

The term “pPCB15” refers to the plasmid (FIG. 7; SEQ ID NO:64) containing β-carotene synthesis genes Pantoea crtEXYIB, using as a reporter plasmid for monitoring β-carotene production in E. coli genetically engineered via the present method.

The term “pKD46” refers to the plasmid (SEQ ID NO:65; Datsenko and Wanner, supra) having GenBank® Accession number AY048746. Plasmid pKD46 expresses the components of the λ-Red Recombinase system.

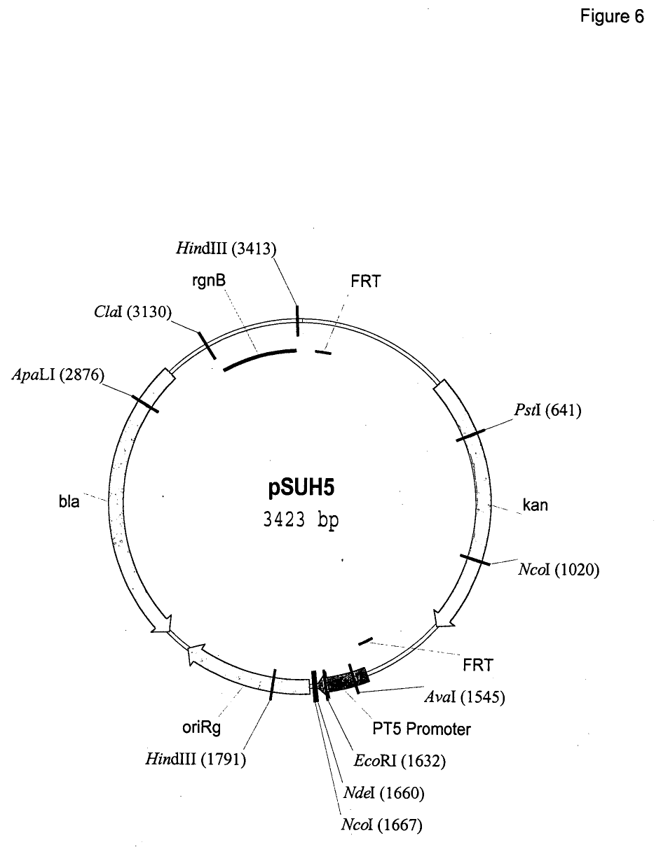

The term “pSUH5” refers to the plasmid (FIG. 6; SEQ ID NO:66) that was constructed by cloning a phage T5 promoter (P_{T5}) region into the NdeI restriction endonuclease site of pKD4 (Datsenko and Wanner, supra). It was used as a template plasmid for PCR amplification of a fused kanamycin selectable marker/phage T5 promoter linear DNA nucleotide.

The term “triple homologous recombination” in the present invention refers to a genetic recombination between two linear (PCR-generated) DNA fragments and the target chromosome via their homologous sequences resulting in chromosomal integration of the two linear nucleic acid fragments into the target chromosome.

The term “homology arm” refers to a nucleotide sequence which enables homologous recombination between two nucleic acids having substantially the same nucleotide sequence in a particular region of two different nucleic acids. The preferred size range of the nucleotide sequence of the homology arm is from about 10 to about 100 nucleotides.

The term “site-specific recombinase” is used in the present invention to describe a system comprised of one or more enzymes which recognize specific nucleotide sequences (recombination target sites) and which catalyze recombination between the recombination target sites. Site-specific recombination provides a method to rearrange, delete, or introduce exogenous DNA. Examples of site-specific recombinases and their associated recombination target sites are: Cre-lox, FLP/FRT, R/RS, Gin/gix, Xer/dif, In/att, a pSR1 system, a cer system, and a fim system. The present invention illustrates the use of a site-specific recombinase to remove selectable markers. Antibiotic resistance markers, flanked on both sides by FRT recombination target sites, are removed by expression of the FLP site-specific recombinase.

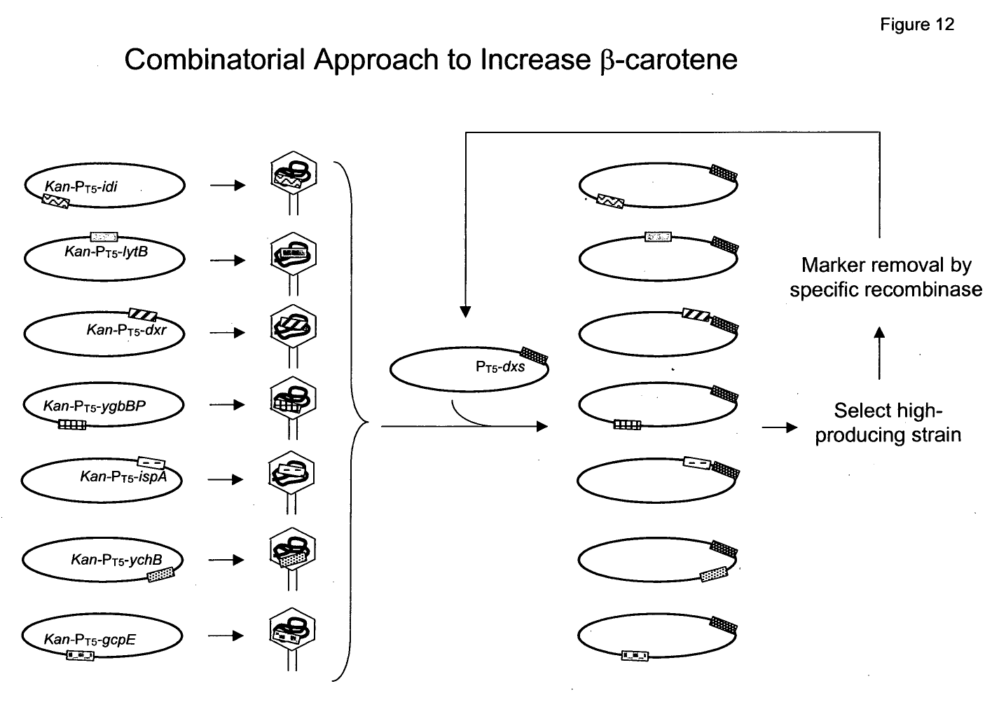

The terms “stacking”, “combinatorial stacking”, “chromosomal stacking”, and “trait stacking” are used interchangeably and refer to the repeated process of stacking multiple genetic traits into one E. coli host using bacteriophage P1 transduction in combination with the site-specific recombinase system for removal of selection markers (FIG. 12).

The term “parallel combinatorial fashion” refers to the P1 transduction with the P1 lysate mixture made from various donor cells, so that multiple genetic traits can move the recipient cell in parallel.

The term “integration cassette” and “recombination element” refers to a linear nucleic acid construct useful for the transformation of a recombination proficient bacterial host. Recombination elements of the invention may include a variety of genetic elements such as selectable markers, expressible DNA fragments, and recombination regions having homology to regions on a bacterial chromosome or on other recombination elements. Expressible DNA fragments can include promoters, coding sequences, genes, and other regulatory elements specifically engineered into the recombination element to impart a desired phenotypic change upon recombination.

The term “expressible DNA fragment” means any DNA that influences phenotypic changes in the host cell. An “expressible DNA fragment” may include for example, DNA comprising regulatory elements, isolated promoters, open reading frames, coding sequences, genes, or combinations thereof.

The term “pDCQ108” refers to the plasmid containing β-carotene synthesis genes Pantoea crtEXYIB used as a reporter plasmid for monitoring β-carotene production in E. coli that were genetically engineered via the present method (ATCC PTA-4823).

The terms “P_{T5} promoter” and “phage T5 promoter” are used interchangeably and refer to the nucleotide sequence that comprises the −10 and −35 consensus sequences, lactose operator (lacO), and ribosomal binding site (rbs) from phage T5 (SEQ ID NO:33).

The term “helper plasmid” refers to either pKD46 encoding λ-Red recombinase or pCP20 encoding FLP site-specific recombinase (ATCC PTA-4455; Datsenko and Wanner, supra; and Cherepanov and Wackernagel, Gene, 158:9-14 (1995)).

The term “carotenoid overproducing bacteria” refers to a bacteria of the invention which has been genetically modified by the up-regulation or down-regulation of various genes to produce a carotenoid compound a levels greater than the wildtype or unmodified host.

The term “E. coli” refers to Escherichia coli strain K-12 derivatives, such as MG1655 (ATCC 47076) and MC1061 (ATCC 53338).

The term “Pantoea stewartii subsp. stewartii” is abbreviated as “Pantoea stewartii” and is used interchangeably with Erwinia stewartii (Mergaert et al., Int J. Syst. Bacteriol., 43:162-173 (1993)).

The term “Pantoea ananatas” is used interchangeably with Erwinia uredovora (Mergaert et al., supra).

The term “Pantoea crtEXYIB cluster” refers to a gene cluster containing carotenoid synthesis genes crtEXYIB amplified from Pantoea stewartii ATCC 8199. The gene cluster contains the genes crtE, crtX, crtY, crtI, and crtB. The cluster also contains a crtZ gene organized in opposite orientation and adjacent to crtB gene.

The term “CrtE” refers to geranylgeranyl pyrophosphate synthase enzyme encoded by crtE gene which converts trans-trans-farnesyl diphosphate+isopentenyl diphosphate to pyrophosphate+geranylgeranyl diphosphate.

The term “CrtY” refers to lycopene cyclase enzyme encoded by crtY gene which converts lycopene to β-carotene.

The term “CrtI” refers to phytoene dehydrogenase enzyme encoded by crtI gene which converts phytoene into lycopene via the intermediaries of phytofluene, zeta-carotene and neurosporene by the introduction of 4 double bonds

The term “CrtB” refers to phytoene synthase enzyme encoded by crtB gene which catalyzes reaction from prephytoene diphosphate (geranylgeranyl pyrophosphate) to phytoene.

The term “CrtX” refers to zeaxanthin glucosyl transferase enzyme encoded by crtX gene which converts zeaxanthin to zeaxanthin-β-diglucoside.

The term “CrtZ” refers to the β-carotene hydroxylase enzyme encoded by crtZ gene which catalyses hydroxylation reaction from β-carotene to zeaxanthin.

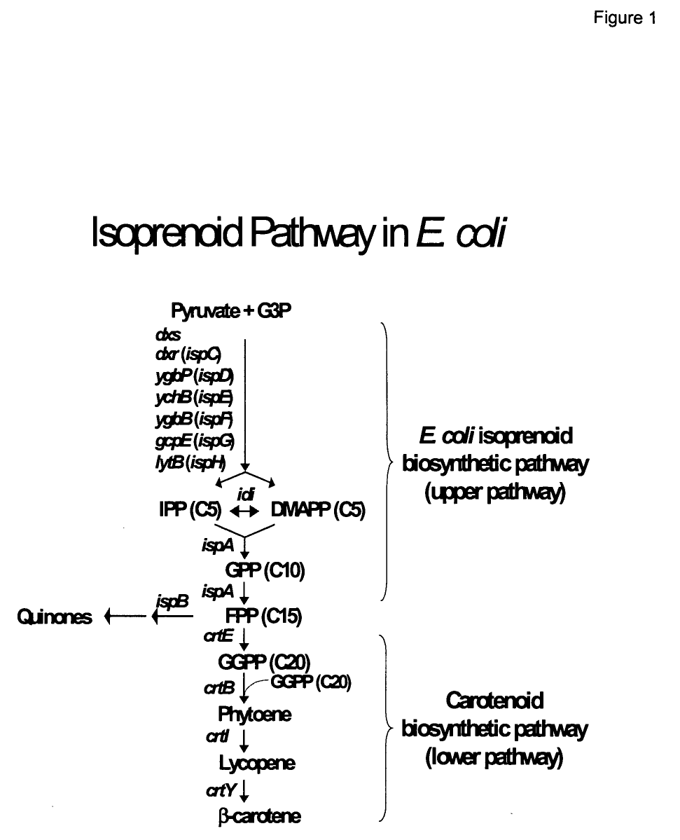

The term “carotenoid biosynthetic pathway” refers to those genes comprising members of the upper and/or lower isoprenoid pathways of the present invention as illustrated in FIG. 1. In the present invention, the terms “upper isoprenoid pathway” and “upper pathway” will be use interchangeably and will refer the enzymes involved in converting pyruvate and glyceraldehyde-3-phosphate to farnesyl pyrophosphate (FPP). These enzymes include, but are not limited to Dxs, Dxr (IspC), YgpP (IspD), YchB (IspE), YgbB (IspF), GcpE (IspG), LytB (IspH), Idi, IspA, and optionally IspB. In the present invention, the terms “lower carotenoid pathway” and “lower pathway” will be used interchangeably and refer to those enzymes which convert FPP to carotenoids, especially β-carotene (FIG. 1). The enzymes in this pathway include, but are not limited to CrtE, CrtY, CrtI, CrtB, CrtX, and CrtZ. In the present invention, the “lower pathway” genes are expressed on reporter plasmids pPCB15 or pDCQ108.

The term “carotenoid biosynthetic enzyme” is an inclusive term referring to any and all of the enzymes encoded by the Pantoea crtEXYIB cluster. The enzymes include CrtE, CrtY, CrtI, CrtB, and CrtX.

The terms “P1 donor cell” and “donor cell” are used interchangeably in the present invention and refer to a bacterial strain susceptible to infection by a bacteriophage or virus, and which serves as a source for the nucleic acid fragments packaged into the transducing particles. Typically the genetic make up of the donor cell is similar or identical to the “recipient cell” which serves to receive P1 lysate containing transducing particles or virus produced by the donor cell.

The terms “P1 recipient cell” and “recipient cell” are used interchangeably in the present invention and refer to a bacterial strain susceptible to infection by a bacteriophage or virus and which serves to receive lysate containing transducing particles or virus produced by the donor cell.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “genetic end product” means the substance, chemical or material (i.e. isoprenoids, carotenoids) that is produced as the result of the activity of a gene product. Typically a gene product is an enzyme and a genetic end product is the product of that enzymatic activity on a specific substrate. A genetic end product may the result of a single enzyme activity or the result of a number of linked activities, such as found in a biosynthetic pathway (several enzyme activites).

“Operon”, in bacterial DNA, is a cluster of contiguous genes transcribed from one promoter that gives rise to a polycistronic mRNA.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site(s), effector binding site(s), and stem-loop structure(s).

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions (“inducible promoters”). Promoters which cause a gene to be expressed in most cell types at most times are commonly referred to as “constitutive promoters”. Promoters can be further classified by the relative strength of expression observed by their use (i.e. weak, moderate, or strong). It is further recognized that since in most cases the exact boundaries of regulatory sequences have not been completely defined, DNA fragments of different lengths may have identical promoter activity.

The “3′ non-coding sequences” refer to DNA sequences located downstream of a coding sequence and include regulatory signals capable of affecting mRNA processing or gene expression.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “Sense” RNA refers to RNA transcript that includes the mRNA and so can be translated into protein by the cell. “Antisense RNA” refers to an RNA transcript that is complementary to all or part of a target primary transcript or mRNA and that blocks the expression of a target gene (U.S. Pat. No. 5,107,065; WO 99/28508). The complementarity of an antisense RNA may be with any part of the specific gene transcript, i.e., at the 5′ non-coding sequence, 3′ non-coding sequence, or the coding sequence.

“Functional RNA” refers to antisense RNA, ribozyme RNA, or other RNA that is not translated yet has an effect on cellular processes.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide.

“Transformation” refers to the transfer of a nucleic acid fragment into the genome of a host organism, resulting in genetically stable inheritance. Host organisms containing the transformed nucleic acid fragments are referred to as “transgenic”, “recombinant” or “transformed” organisms.

The terms “transduction” and “generalized transduction” are used interchangeably and refer to a phenomenon in which bacterial DNA is transferred from one bacterial cell (the donor) to another (the recipient) by a phage particle containing bacterial DNA (FIG. 12). The bacterial DNA fragment from the donor can undergo homologous recombination with the recipient cell's chromosome, stably integrating the donor cell's DNA fragment into the recipient's chromosome.

The terms “plasmid”, “vector” and “cassette” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitates transformation of a particular host cell. “Expression cassette” refers to a specific vector containing a foreign gene and having elements in addition to the foreign gene that allow for enhanced expression of that gene in a foreign host.

The term “sequence analysis software” refers to any computer algorithm or software program that is useful for the analysis of nucleotide or amino acid sequences. “Sequence analysis software” may be commercially available or independently developed. Typical sequence analysis software will include but is not limited to the GCG suite of programs (Wisconsin Package Version 9.0, Genetics Computer Group (GCG), Madison, WI), BLASTP, BLASTN, BLASTX (Altschul et al., J. Mol. Biol. 215:403-410 (1990), and DNASTAR (DNASTAR, Inc. 1228 S. Park St. Madison, Wis. 53715 USA), and the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

The present invention relates to carotenoid overproducing bacteria. The genes of the isoprenoid pathway in the bacterial hosts of the invention have been engineered such that certain genes are either up-regulated or down regulated resulting in the production of carotenoid compounds at a higher level than is found in the unmodified host. In some instances the genes that are regulated are directly involved in the carotenoid biosynthetic pathway. In other instances the genes involved are chromosomal genes that have no understood relationship to the carotenoid biosynthetic pathway.

It has been found that over-expression of certain combinations of carotenoid biosynthetic genes will give an unexpectedly high level of carotenoid production. Examples of genes useful in this manner which are part of the carotenoid biosynthetic pathway are the dxs gene, (catalyzing the condensation of pyruvate and D-glyceraldehyde 3-phosphate to D-1-deoxyxylulose 5-phosphate), the idi gene (converting isopentenyl diphosphate to dimethylallyl diphosphate), the ygbB (ispF) gene (catalyzing the cyclization with loss of CMP of 4-diphophocytidyl-2C-methyl-D-erythritol to 4-diphosphocytidyl-2C-methyl-D-erythritol-2-phosphate to 2C-methyl-D-erythritol-2,4-cyclodiphosphate), the ygbP (ispD) gene (catalyzeing the CTP-dependent cytidylation of 2-C-methyl-D-erythritol-4-phosphate to 4-diphophocytidyl-2C-methyl-D-erythritol) and together referred to as the ygbBP gene, the lytB (ispH) gene (involved in conversion of 2C-methyl-D-erythritol-2,4-cyclodiphosphate to dimethylallyl diphosphate and isopentenyl diphosphate), and the ispB gene encoding the enzyme octaprenyl diphosphate synthase. When these genes are selectively over expressed under the control of a strong promoter the result is an unexpectedly high level of carotenoid production. It is important to note that it is the combination of the over-expression of these genes that has been shown to give the desired effect.

Alternatively, it has also been found that certain essential chromosomal genes, when mutated, will alter the output of the carotenoid biosynthetic pathway. One such gene is the yjeR gene (defining a oligo-ribonuclease locus). It has been found that a partial mutation in this gene will unexpectedly increase carotenoid production in a host cell capable of cartenoid biosynthesis.

Genes Involved in Carotenoid Production.

The enzyme pathway involved in the biosynthesis of carotenoids can be conveniently viewed in two parts, the upper isoprenoid pathway providing for the conversion of pyruvate and glyceraldehyde-3-phosphate to farnesyl pyrophosphate (FPP) and the lower carotenoid biosynthetic pathway, which provides for the synthesis of phytoene and all subsequently produced carotenoids. The upper pathway is ubiquitous in many non-carotogenic microorganisms and in these cases it will only be necessary to introduce genes that comprise the lower pathway for the biosynthesis of the desired carotenoid. The key division between the two pathways concerns the synthesis of farnesyl pyrophosphate. Where FPP is naturally present, only elements of the lower carotenoid pathway will be needed. However, it will be appreciated that for the lower pathway carotenoid genes to be effective in the production of carotenoids, it will be necessary for the host cell to have suitable levels of FPP within the cell. Where FPP synthesis is not provided by the host cell, it will be necessary to introduce the genes necessary for the production of FPP. Each of these pathways will be discussed below in detail.

The Upper Isoprenoid Pathway

Isoprenoid biosynthesis occurs through either of two pathways, generating the common C5 isoprene sub-unit, isopentenyl pyrophosphate (IPP). First, IPP may be synthesized through the well-known acetate/mevalonate pathway. However, recent studies have demonstrated that the mevalonate-dependent pathway does not operate in all living organisms. An alternate mevalonate-independent pathway for IPP biosynthesis has been characterized in bacteria and in green algae and higher plants (Horbach et al., FEMS Microbiol. Lett., 111:135-140 (1993); Rohmer et al., Biochem., 295: 517-524 (1993); Schwender et al., Biochem., 316: 73-80 (1996); and Eisenreich et al., Proc. Natl. Acad. Sci. USA, 93: 6431-6436 (1996)).

Many steps in the mevalonate-independent isoprenoid pathway are known (FIG. 1). For example, the initial steps of the alternate pathway leading to the production of IPP have been studied in Mycobacterium tuberculosis by Cole et al. (Nature, 393:537-544 (1998)). The first step of the pathway involves the condensation of two 3-carbon molecules (pyruvate and D-glyceraldehyde 3-phosphate) to yield a 5-carbon compound known as D-1-deoxyxylulose-5-phosphate. This reaction occurs by the DXS enzyme, encoded by the dxs gene. Next, the isomerization and reduction of D-1-deoxyxylulose-5-phosphate yields 2-C-methyl-D-erythritol-4-phosphate. One of the enzymes involved in the isomerization and reduction process is D-1-deoxyxylulose-5-phosphate reductoisomerase (DXR), encoded by the gene dxr (ispC). 2-C-methyl-D-erythritol-4-phosphate is subsequently converted into 4-diphosphocytidyl-2C-methyl-D-erythritol in a CTP-dependent reaction by the enzyme encoded by the non-annotated gene ygbP. Recently, however, the ygbP gene was renamed as ispD as a part of the isp gene cluster (SwissProtein Accession #Q46893).

Next, the 2nd position hydroxy group of 4-diphosphocytidyl-2C-methyl-D-erythritol can be phosphorylated in an ATP-dependent reaction by the enzyme encoded by the ychB gene. YchB phosphorylates 4-diphosphocytidyl-2C-methyl-D-erythritol, resulting in 4-diphosphocytidyl-2C-methyl-D-erythritol 2-phosphate. The ychB gene was renamed as ispE, also as a part of the isp gene cluster (SwissProtein Accession #P24209). YgbB converts 4-diphosphocytidyl-2C-methyl-D-erythritol 2-phosphate to 2C-methyl-D-erythritol 2,4-cyclodiphosphate in a CTP-dependent manner. This gene has also been recently renamed, and belongs to the isp gene cluster. Specifically, the new name for the ygbB gene is ispF (SwissProtein Accession #P36663).

The enzymes encoded by the gcpE (ispG) and lytB (ispH) genes (and perhaps others) are thought to participate in the reactions leading to formation of isopentenyl pyrophosphate (IPP) and dimethylallyl pyrophosphate (DMAPP). IPP may be isomerized to DMAPP via IPP isomerase, encoded by the idi gene. However, this enzyme is not essential for survival and may be absent in some bacteria using 2-C-methyl-D-erythritol 4-phosphate (MEP) pathway. Recent evidence suggests that the MEP pathway branches before IPP and separately produces IPP and DMAPP via the lytB gene product. A lytB knockout mutation is lethal in E. coli except in media supplemented with both IPP and DMAPP.

The synthesis of FPP occurs via the isomerization of IPP to dimethylallyl pyrophosphate. This reaction is followed by a sequence of two prenyltransferase reactions catalyzed by ispA, leading to the creation of geranyl pyrophosphate (GPP; a 10-carbon molecule) and farnesyl pyrophosphate (FPP; a 15-carbon molecule).

Genes encoding elements of the upper pathway are known from a variety of plant, animal, and bacterial sources, as shown in Table 1.

TABLE 1
Sources of Genes Encoding the Upper Isoprene Pathway

| | GenBank Accession Number and |
|---|---|
| Gene | Source Organism |
| dxs (D-1- | AF035440, Escherichia coli |
| deoxyxylulose 5- | Y18874, Synechococcus PCC6301 |
| phosphate | AB026631, Streptomyces sp. CL190 |
| synthase) | AB042821, Streptomyces griseolosporeus |
| | AF111814, Plasmodium falciparum |
| | AF143812, Lycopersicon esculentum |
| | AJ279019, Narcissus pseudonarcissus |
| | AJ291721, Nicotiana tabacum |
| dxr (ispC) (1- | AB013300, Escherichia coli |
| deoxy-D- | AB049187, Streptomyces griseolosporeus |
| xylulose 5- | AF111813, Plasmodium falciparum |
| phosphate | AF116825, Mentha x piperita |
| reductoisomerase) | AF148852, Arabidopsis thaliana |
| | AF182287, Artemisia annua |
| | AF250235, Catharanthus roseus |
| | AF282879, Pseudomonas aeruginosa |
| | AJ242588, Arabidopsis thaliana |
| | AJ250714, Zymomonas mobilis strain ZM4 |
| | AJ292312, Klebsiella pneumoniae, |
| | AJ297566, Zea mays |
| ygbP (ispD) (2- | AB037876, Arabidopsis thaliana |
| C-methyl-D- | AF109075, Clostridium difficile |
| erythritol 4- | AF230736, Escherichia coli |
| phosphate | AF230737, Arabidopsis thaliana |
| cytidylyltransferase) | |
| ychB (ispE) (4- | AF216300, Escherichia coli |
| diphosphocytidyl- | AF263101, Lycopersicon esculentum |
| 2-C-methyl-D- | AF288615, Arabidopsis thaliana |
| erythritol kinase) | |
| ygbB (ispF) (2- | AB038256, Escherichia coli mecs gene |
| C-methyl-D- | AF230738, Escherichia coli |
| erythritol 2,4- | AF250236, Catharanthus roseus (MECS) |
| cyclodiphosphate | AF279661, Plasmodium falciparum |
| synthase) | AF321531, Arabidopsis thaliana |
| gcpE (ispG) (1- | O67496, Aquifex aeolicus |
| hydroxy-2- | P54482, Bacillus subtilis |
| methyl-2-(E)- | Q9pky3, Chlamydia muridarum |
| butenyl 4- | Q9Z8H0, Chlamydophila pneumoniae |
| diphosphate | O84060, Chlamydia trachomatis |
| synthase) | P27433, Escherichia coli |
| | P44667, Haemophilus influenzae |
| | Q9ZLL0, Helicobacter pylori J99 |
| | O33350, Mycobacterium tuberculosis |
| | S77159, Synechocystis sp. |
| | Q9WZZ3, Thermotoga maritima |
| | O83460, Treponema pallidum |
| | Q9JZ40, Neisseria meningitidis |
| | Q9PPM1, Campylobacter jejuni |
| | Q9RXC9, Deinococcus radiodurans |
| | AAG07190, Pseudomonas aeruginosa |
| | Q9KTX1, Vibrio cholerae |
| lytB (ispH) | AF027189, Acinetobacter sp. BD413 |
| | AF098521, Burkholderia pseudomallei |
| | AF291696, Streptococcus pneumoniae |
| | AF323927, Plasmodium falciparum gene |
| | M87645, Bacillus subtillis |
| | U38915, Synechocystis sp. |
| | X89371, C. jejunisp O67496 |
| lspA (FPP | AB003187, Micrococcus luteus |
| synthase) | AB016094, Synechococcus elongatus |
| | AB021747, Oryza sativa FPPS1 gene for farnesyl |
| | diphosphate synthase |
| | AB028044, Rhodobacter sphaeroides |
| | AB028046, Rhodobacter capsulatus |
| | AB028047, Rhodovulum sulfidophilum |
| | AF112881 and AF136602, Artemisia annua |
| | AF384040, Mentha x piperita |
| | D00694, Escherichia coli |
| | D13293, B. stearothermophilus |
| | D85317, Oryza sativa |
| | X75789, A. thaliana |
| | Y12072, G. arboreum |
| | Z49786, H. brasiliensis |
| | U80605, Arabidopsis thaliana farnesyl diphosphate |
| | synthase precursor (FPS1) mRNA, complete cds |
| | X76026, K. lactis FPS gene for farnesyl |
| | diphosphate synthetase, QCR8 gene for bc1 |
| | complex, subunit VIII X82542, P. argentatum |
| | mRNA for farnesyl diphosphate synthase |
| | (FPS1) X82543, P. argentatum mRNA for |
| | farnesyl diphosphate synthase (FPS2) |
| | BC010004, Homo sapiens, farnesyl diphosphate |
| | synthase (farnesyl pyrophosphate synthetase, |
| | dimethylallyltranstransferase, |
| | geranyltranstransferase), clone MGC 15352 |
| | IMAGE, 4132071, mRNA, complete cds |
| | AF234168, Dictyostelium discoideum farnesyl |
| | diphosphate synthase (Dfps) |
| | L46349, Arabidopsis thaliana farnesyl diphosphate |
| | synthase (FPS2) mRNA, complete cds |
| | L46350, Arabidopsis thaliana farnesyl diphosphate |
| | synthase (FPS2) gene, complete cds |
| | L46367, Arabidopsis thaliana farnesyl diphosphate |
| | synthase (FPS1) gene, alternative products, |
| | complete cds |
| | M89945, Rat farnesyl diphosphate synthase gene, |
| | exons 1-8 |
| | NM_002004, Homo sapiens farnesyl diphosphate |
| | synthase (farnesyl pyrophosphate synthetase, |
| | dimethylallyltranstransferase, |
| | geranyltranstransferase) (FDPS), mRNA |
| | U36376, Artemisia annua farnesyl diphosphate |
| | synthase (fps1) mRNA, complete cds |
| | XM_001352, Homo sapiens farnesyl diphosphate |
| | synthase (farnesyl pyrophosphate synthetase, |
| | dimethylallyltranstransferase, |
| | geranyltranstransferase) (FDPS), mRNA |
| | XM_034497, Homo sapiens farnesyl diphosphate |
| | synthase (farnesyl pyrophosphate synthetase, |
| | dimethylallyltranstransferase, |
| | geranyltranstransferase) (FDPS), mRNA |
| | XM_034498, Homo sapiens farnesyl diphosphate |
| | synthase (farnesyl pyrophosphate synthetase, |
| | dimethylallyltranstransferase, |
| | geranyltranstransferase) (FDPS), mRNA |
| | XM_034499, Homo sapiens farnesyl diphosphate |
| | synthase (farnesyl pyrophosphate synthetase, |
| | dimethylallyltranstransferase, |
| | geranyltranstransferase) (FDPS), mRNA |
| | XM_0345002, Homo sapiens farnesyl diphosphate |
| | synthase (farnesyl pyrophosphate synthetase, |
| | dimethylallyltranstransferase, |
| | geranyltranstransferase) (FDPS), mRNA |

The most preferred source of genes for the upper isoprene pathway in the present invention is from Methylomonas 1 6a (ATCC PTA-2402). Methylomonas 16a is particularly well-suited for the present invention, as the methanotroph is naturally pink-pigmented, producing a 30-carbon carotenoid. Thus, the organism possesses the genes of the upper isoprene pathway. Sequences of these preferred genes are presented as the following SEQ ID numbers: the dxs(16a) gene (SEQ ID NO:13), the dxr(16a) gene (SEQ ID NO:17), and the lytB(16a) gene (SEQ ID NO:15).

The Lower Carotenoid Biosynthetic Pathway

The division between the upper isoprenoid pathway and the lower carotenoid pathway is somewhat subjective. Because FPP synthesis is common in both carotenogenic and non-carotenogenic bacteria, the first step in the lower carotenoid biosynthetic pathway is considered to begin with the prenyltransferase reaction converting farnesyl pyrophosphate (FPP) to geranylgeranyl pyrophosphate (GGPP). The gene crtE, encoding GGPP synthetase, is responsible for this prenyltransferase reaction which adds IPP to FPP to produce the 20-carbon molecule GGPP. A condensation reaction of two molecules of GGPP occurs to form phytoene (PPPP), the first 40-carbon molecule of the lower carotenoid biosynthesis pathway. This enzymatic reaction is catalyzed by crtB, encoding phytoene synthase.

Lycopene, which imparts a “red” colored spectra, is produced from phytoene through four sequential dehydrogenation reactions by the removal of eight atoms of hydrogen, catalyzed by the gene crtI (encoding phytoene desaturase). Intermediaries in this reaction are phytofluene, zeta-carotene, and neurosporene.

Lycopene cyclase (crtY) converts lycopene to β-carotene. In the present invention, a reporter plasmid is used which produces β-carotene as the genetic end product. However, additional genes may be used to create a variety of other carotenoids. For example, β-carotene is converted to zeaxanthin via a hydroxylation reaction resulting from the activity of β-carotene hydroxylase (encoded by the crtZ gene). β-cryptoxanthin is an intermediate in this reaction.

β-carotene is converted to canthaxanthin by β-carotene ketolase encoded by either the crtW or crtO gene. Echinenone in an intermediate in this reaction. Canthaxanthin can then be converted to astaxanthin by β-carotene hydroxylase encoded by the crtZ or crtR gene. Adonbirubrin is an intermediate in this reaction.

Zeaxanthin can be converted to zeaxanthin-β-diglucoside. This reaction is catalyzed by zeaxanthin glucosyl transferase (crtX).

Zeaxanthin can be converted to astaxanthin by β-carotene ketolase encoded by crtW, crtO or bkt. The BKT/CrtW enzymes synthesized canthaxanthin via echinenone from β-carotene and 4-ketozeaxanthin. Adonixanthin is an intermediate in this reaction.

Spheroidene can be converted to spheroidenone by spheroidene monooxygenase encoded by crtA.

Neurosporene can be converted spheroidene and lycopene can be converted to spirilloxanthin by the sequential actions of hydroxyneurosporene synthase, methoxyneurosporene desaturase and hydroxyneurosporene-O-methyltransferase encoded by the crtC, crtD and crtF genes, respectively.

β-carotene can be converted to isorenieratene by β-carotene desaturase encoded by crtU.

Genes encoding elements of the lower carotenoid biosynthetic pathway are known from a variety of plant, animal, and bacterial sources, as shown in Table 2.

TABLE 2
Sources of Genes Encoding the Lower Carotenoid Biosynthetic Pathway

| | GenBank Accession Number and |
|---|---|
| Gene | Source Organism |
| crtE (GGPP | AB000835, Arabidopsis thaliana |
| Synthase) | AB016043 and AB019036, Homo sapiens |
| | AB016044, Mus musculus |
| | AB027705 and AB027706, Daucus carota |
| | AB034249, Croton sublyratus |
| | AB034250, Scoparia dulcis |
| | AF020041, Helianthus annuus |
| | AF049658, Drosophila melanogaster signal |
| | recognition particle 19 kDa protein (srp19) gene, partial |
| | sequence; and geranylgeranyl pyrophosphate |
| | synthase (quemao) gene, complete cds |
| | AF049659, Drosophila melanogaster geranylgeranyl |
| | pyrophosphate synthase mRNA, complete cds |
| | AF139916, Brevibacterium linens |
| | AF279807, Penicillium paxilli geranylgeranyl |
| | pyrophosphate synthase (ggs1) gene, complete |
| | AF279808, Penicillium paxilli dimethylallyl tryptophan |
| | synthase (paxD) gene, partial cds; and cytochrome |
| | P450 monooxygenase (paxQ), cytochrome P450 |
| | monooxygenase (paxP), PaxC (paxC), |
| | monooxygenase (paxM), geranylgeranyl |
| | pyrophosphate synthase (paxG), PaxU (paxU), and |
| | metabolite transporter (paxT) genes, complete cds |
| | AJ010302, Rhodobacter sphaeroides |
| | AJ133724, Mycobacterium aurum |
| | AJ276129, Mucor circinelloides f. lusitanicus carG |
| | gene for geranylgeranyl pyrophosphate synthase, |
| | exons 1-6 |
| | D85029, Arabidopsis thaliana mRNA for |
| | geranylgeranyl pyrophosphate synthase, partial cds |
| | L25813, Arabidopsis thaliana |
| | L37405, Streptomyces griseus geranylgeranyl |
| | pyrophosphate synthase (crtB), phytoene desaturase |
| | (crtE) and phytoene synthase (crtI) genes, complete cds |
| | U15778, Lupinus albus geranylgeranyl |
| | pyrophosphate synthase (ggps1) mRNA, complete cds |
| | U44876, Arabidopsis thaliana pregeranylgeranyl |
| | pyrophosphate synthase (GGPS2) mRNA, complete cds |
| | X92893, C. roseus |
| | X95596, S. griseus |
| | X98795, S. alba |
| | Y15112, Paracoccus marcusii |
| crtX (Zeaxanthin | D90087, E. uredovora |
| glucosylase) | M87280 and M90698, Pantoea agglomerans |
| crtY (Lycopene-β- | AF139916, Brevibacterium linens |
| cyclase) | AF152246, Citrus x paradisi |
| | AF218415, Bradyrhizobium sp. ORS278 |
| | AF272737, Streptomyces griseus strain IFO13350 |
| | AJ133724, Mycobacterium aurum |
| | AJ250827, Rhizomucor circinelloides f. lusitanicus |
| | carRP gene for lycopene cyclase/phytoene synthase, exons 1-2 |
| | AJ276965, Phycomyces blakesleeanus carRA gene |
| | for phytoene synthase/lycopene cyclase, exons 1-2 |
| | D58420, Agrobacterium aurantiacum |
| | D83513, Erythrobacter longus |
| | L40176, Arabidopsis thaliana lycopene cyclase |
| | (LYC) mRNA, complete cds |
| | M87280, Pantoea agglomerans |
| | U50738, Arabodopsis thaliana lycopene epsilon |
| | cyclase mRNA, complete cds |
| | U50739, Arabidosis thaliana lycopene β cyclase |
| | mRNA, complete cds |
| | U62808, Flavobacterium ATCC21588 |
| | X74599, Synechococcus sp. Icy gene for lycopene cyclase |
| | X81787, N. tabacum CrtL-1 gene encoding lycopene cyclase |
| | X86221, C. annuum |
| | X86452, L. esculentum mRNA for lycopene β-cyclase |
| | X95596, S. griseus |
| | X98796, N. pseudonarcissus |
| crtI (Phytoene | AB046992, Citrus unshiu CitPDS1 mRNA for |
| desaturase) | phytoene desaturase, complete cds |
| | AF039585, Zea mays phytoene desaturase (pds1) |
| | gene promoter region and exon 1 |
| | AF049356, Oryza sativa phytoene desaturase |
| | precursor (Pds) mRNA, complete cds |
| | AF139916, Brevibacterium linens |
| | AF218415, Bradyrhizobium sp. ORS278 |
| | AF251014, Tagetes erecta |
| | AF364515, Citrus x paradisi |
| | D58420, Agrobacterium aurantiacum |
| | D83514, Erythrobacter longus |
| | L16237, Arabidopsis thaliana |
| | L37405, Streptomyces griseus geranylgeranyl |
| | pyrophosphate synthase (crtB), phytoene desaturase |
| | (crtE) and phytoene synthase (crtI) genes, complete cds |
| | L39266, Zea mays phytoene desaturase (Pds) |
| | mRNA, complete cds |
| | M64704, Soybean phytoene desaturase |
| | M88683, Lycopersicon esculentum phytoene |
| | desaturase (pds) mRNA, complete cds |
| | S71770, carotenoid gene cluster |
| | U37285, Zea mays |
| | U46919, Solanum lycopersicum phytoene desaturase |
| | (Pds) gene, partial cds |
| | U62808, Flavobacterium ATCC21588 |
| | X55289, Synechococcus pds gene for phytoene desaturase |
| | X59948, L. esculentum |
| | X62574, Synechocystis sp. pds gene for phytoene desaturase |
| | X68058, C. annuum pds1 mRNA for phytoene desaturase |
| | X71023, Lycopersicon esculentum pds gene for |
| | phytoene desaturase |
| | X78271, L. esculentum (Ailsa Craig) PDS gene |
| | X78434, P. blakesleeanus (NRRL1555) carB gene |
| | X78815, N. pseudonarcissus |
| | X86783, H. pluvialis |
| | Y14807, Dunaliella bardawil |
| | Y15007, Xanthophyllomyces dendrorhous |
| | Y15112, Paracoccus marcusii |
| | Y15114, Anabaena PCC7210 crtP gene |
| | Z11165, R. capsulatus |
| crtB (Phytoene | AB001284, Spirulina platensis |
| synthase) | AB032797, Daucus carota PSY mRNA for phytoene |
| | synthase, complete cds |
| | AB034704, Rubrivivax gelatinosus |
| | AB037975, Citrus unshiu |
| | AF009954, Arabidopsis thaliana phytoene synthase |
| | (PSY) gene, complete cds |
| | AF139916, Brevibacterium linens |
| | AF152892, Citrus x paradisi |
| | AF218415, Bradyrhizobium sp. ORS278 |
| | AF220218, Citrus unshiu phytoene synthase (Psy1) |
| | mRNA, complete cds |
| | AJ010302, Rhodobacter |
| | AJ133724, Mycobacterium aurum |
| | AJ278287, Phycomyces blakesleeanus carRA gene |
| | for lycopene cyclase/phytoene synthase, |
| | AJ304825, Helianthus annuus mRNA for phytoene |
| | synthase (psy gene) |
| | AJ308385, Helianthus annuus mRNA for phytoene |
| | synthase (psy gene) |
| | D58420, Agrobacterium aurantiacum |
| | L23424, Lycopersicon esculentum phytoene synthase |
| | (PSY2) mRNA, complete cds |
| | L25812, Arabidopsis thaliana |
| | L37405, Streptomyces griseus geranylgeranyl |
| | pyrophosphate synthase (crtB), phytoene desaturase |
| | (crtE) and phytoene synthase (crtl) genes, complete cds |
| | M38424, Pantoea agglomerans phytoene synthase |
| | (crtE) gene, complete cds |
| | M87280, Pantoea agglomerans |
| | S71770, Carotenoid gene cluster |
| | U32636, Zea mays phytoene synthase (Y1) gene, |
| | complete cds |
| | U62808, Flavobacterium ATCC21588 |
| | U87626, Rubrivivax gelatinosus |
| | U91900, Dunaliella bardawil |
| | X52291, Rhodobacter capsulatus |
| | X60441, L. esculentum GTom5 gene for phytoene synthase |
| | X63873, Synechococcus PCC7942 pys gene for |
| | phytoene synthase |
| | X68017, C. annuum psy1 mRNA for phytoene synthase |
| | X69172, Synechocystis sp. pys gene for phytoene synthase |
| | X78814, N. pseudonarcissus |
| crtZ (β-carotene | D58420, Agrobacterium aurantiacum |
| hydroxylase) | D58422, Alcaligenes sp. |
| | D90087, E. uredovora |
| | M87280, Pantoea agglomerans |
| | U62808, Flavobacterium ATCC21588 |
| | Y15112, Paracoccus marcusil |
| crtW (β-carotene | AF218415, Bradyrhizobium sp. ORS278 |
| ketolase) | D45881, Haematococcus pluvialis |
| | D58420, Agrobacterium aurantiacum |
| | D58422, Alcaligenes sp. |
| | X86782, H. pluvialis |
| | Y15112, Paracoccus marcusii |
| crtO (β-C4- | X86782, H. pluvialis |
| ketolase) | Y15112, Paracoccus marcusii |
| crtU (β-carotene | AF047490, Zea mays |
| dehydrogenase) | AF121947, Arabidopsis thaliana |
| | AF139916, Brevibacterium linens |
| | AF195507, Lycopersicon esculentum |
| | AF272737, Streptomyces griseus strain IFO13350 |
| | AF372617, Citrus x paradisi |
| | AJ133724, Mycobacterium aurum |
| | AJ224683, Narcissus pseudonarcissus |
| | D26095 and U38550, Anabaena sp. |
| | X89897, C. annuum |
| | Y15115, Anabaena PCC7210 crtQ gene |
| crtA (spheroidene | AJ010302, Rhodobacter sphaeroides |
| monooxygenase) | Z11165 and X52291, Rhodobacter capsulatus |
| crtC | AB034704, Rubrivivax gelatinosus |
| (hydroxyneurosporene | AF195122 and AJ010302, Rhodobacter sphaeroides |
| synthase) | AF287480, Chlorobium tepidum |
| | U73944, Rubrivivax gelatinosus |
| | X52291 and Z11165, Rhodobacter capsulatus |
| | Z21955, M. xanthus |
| crtD (carotenoid | AJ010302 and X63204, Rhodobacter sphaeroides |
| 3,4-desaturase) | U73944, Rubrivivax gelatinosus |
| | X52291 and Z11165, Rhodobacter capsulatus |
| crtF | AB034704, Rubrivivax gelatinosus |
| (1-OH-carotenoid | AF288602, Chloroflexus aurantiacus |
| methylase) | AJ010302, Rhodobacter sphaeroides |
| | X52291 and Z11165, Rhodobacter capsulatus |

The most preferred source of crt genes is from Pantoea stewartii. Sequences of these preferred genes are presented as the following SEQ ID numbers: the crtE gene (SEQ ID NO:1), the crtX gene (SEQ ID NO:3), crtY (SEQ ID NO:5), the crtI gene (SEQ ID NO:7), the crtB gene (SEQ ID NO:9) and the crtZ gene (SEQ ID NO:11).

By using various combinations of the genes presented in Table 2 and the preferred genes of the present invention, innumerable different carotenoids and carotenoid derivatives could be made using the methods of the present invention, provided that sufficient sources of FPP are available in the host organism. For example, the gene cluster crtEXYIB enables the production of β-carotene. Addition of the crtZ to crtEXYIB enables the production of zeaxanthin.

It is envisioned that useful products of the present invention will include any carotenoid compound as defined herein including, but not limited to antheraxanthin, adonixanthin, astaxanthin, canthaxanthin, capsorubrin, β-cryptoxanthin, didehydrolycopene, didehydrolycopene, β-carotene, ζ-carotene, δ-carotene, γ-carotene, keto-γ-carotene, γ-carotene, ε-carotene, β,ψ-carotene, torulene, echinenone, gamma-carotene, zeta-carotene, alpha-cryptoxanthin, diatoxanthin, 7,8-didehydroastaxanthin, fucoxanthin, fucoxanthinol, isorenieratene, β-isorenieratene lactucaxanthin, lutein, lycopene, neoxanthin, neurosporene, hydroxyneurosporene, peridinin, phytoene, rhodopin, rhodopin glucoside, siphonaxanthin, spheroidene, spheroidenone, spirilloxanthin, uriolide, uriolide acetate, violaxanthin, zeaxanthin-p-diglucoside, zeaxanthin, and C30-carotenoids.

Methods for Optimizing the Carotenoid Biosynthetic Pathway

Metabolic engineering generally involves the introduction of new metabolic activities into the host organism or the improvement of existing processes by engineering changes such as adding, removing, or modifying genetic elements (Stephanopoulos, G., Metab. Eng., 1: 1-11 (1999)). One such modification is genetically engineering modulations to the expression of relevant genes in a metabolic pathway.

There are a variety of ways to modulate gene expression. Microbial metabolic engineering generally involves the use of multi-copy vectors to express a gene of interest under the control of a constitutive or inducible promoter. This method of metabolic engineering for industrial use has several drawbacks. It is sometimes difficult to maintain the vectors due to segregational instability. Deleterious effects on cell viability and growth are often observed due to the vector burden. It is also difficult to control the optimal expression level of desired genes on a vector. To avoid the undesirable effects of using a multi-copy vector, a chromosomal integration approach using homologous recombination via a single insertion of bacteriophage λ, transposons, or other suitable vectors containing the gene of interest has been used. However, this method also has drawbacks such as the need for multiple cloning steps in order to get the gene of interest into a suitable vector prior to recombination. Another drawback is the instability associated with the inserted genes, which can be lost due to excision. Lastly, these methods have a limitation associated with the number of possible insertions and the inability to control the location of the insertion site on a chromosome.

Several processes are involved in the regulation of gene expression. The main steps are (1) the initiation of transcription, (2) the termination of transcription, (3) the processing of transcripts, and (4) translation. Among these, the transcription initiation is a major step for controlling gene expression. The transcription initiation is determined by the sequence of the promoter region that includes a binding site for RNA polymerase together with possible binding sites for one or more transcription factors.

Strong promoters are widely used for constitutive overexpression of key genes in a metabolic pathway. Strong and moderately strong promoters that are useful for expression in E. coli include lac, trp, λP_{L}, λP_{R}, T7, tac, T5 (P_{T5}), and trc. A conventional way to regulate the amount and the timing of protein expression is to use an inducible promoter. An inducible promoter is not always active the way constitutive promoters are (e.g. viral promoters). Inducible promoters are normally activated in response to certain environmental or chemical stimuli (i.e. heat shock promoter, isopropyl-β-thiogalactopyranoside (IPTG) responsive promoters, and tetracycline (tet) responsive promoters, to name a few).

Promoters of the stationary phase πS regulon, which are active under stress conditions and at the onset of the stationary phase, control expression of about 100 genes involved in the protection of the cell against various stresses. The promoters of the πS regulon genes may also be useful for the expression of the desired genes when the metabolite products inhibit a cell growth. The πS-dependent stationary phase promoters includes rpoS, bolA, appY, dps, cyxAB-appA, csgA, treA, osmB, katE, xthA, otsBA, glgS, osmY, pex, and mcc, to name a few.

Termination control regions may also be derived from various genes native to the preferred hosts. Optionally, a termination site may be unnecessary, however, it is most preferred if included.

Alternatively, it may be necessary to reduce or eliminate the expression of certain genes in the target pathway or in competing pathways that may serve as competing sinks for energy or carbon. Methods of down-regulating genes for this purpose have been explored. Where the sequence of the gene to be disrupted is known, one of the most effective methods of gene down-regulation is targeted gene disruption, a process where foreign DNA is inserted into a structural gene so as to disrupt transcription. This can be effected by the creation of genetic cassettes comprising the DNA to be inserted (often a genetic marker) flanked by sequence having a high degree of homology to a portion of the gene to be disrupted. Introduction of the cassette into the host cell results in insertion of the foreign DNA into the structural gene via the native DNA replication mechanisms of the cell or by the λ-Red recombination system used in the present invention. (See for example Hamilton et al., J. Bacteriol., 171:4617-4622 (1989); Balbas et al., Gene, 136:211-213 (1993); Gueldener et al., Nucleic Acids Res., 24:2519-2524 (1996); and Smith et al., Methods Mol. Cell. Biol., 5:270-277 (1996)) Antisense technology is another method of down regulating genes where the sequence of the target gene is known. To accomplish this, a nucleic acid segment from the desired gene is cloned and operably linked to a promoter such that the anti-sense strand of RNA will be transcribed. This construct is then introduced into the host cell and the antisense strand of RNA is produced. Antisense RNA inhibits gene expression by preventing the accumulation of mRNA which encodes the protein of interest. A person of skill in the art will know that special considerations are associated with the use of antisense technologies in order to reduce expression of particular genes. For example, the proper level of expression of antisense genes may require the use of different chimeric genes utilizing different regulatory elements known to the skilled artisan.

Although targeted gene disruption and antisense technology offer effective means of down regulating genes where the sequence is known, other less specific methodologies have been developed that are not sequence based. For example, cells may be exposed to UV radiation and then screened for the desired phenotype. Mutagenesis with chemical agents is also effective for generating mutants and commonly used substances include chemicals that affect non-replicating DNA such as HNO₂ and NH₂OH, as well as agents that affect replicating DNA such as acridine dyes, notable for causing frame-shift mutations. Specific methods for creating mutants using radiation or chemical agents are well documented in the art. See for example Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36, 227, (1992).

Another non-specific method of gene disruption is the use of transposable elements or transposons. Transposons are genetic elements that insert randomly into DNA but can be latter retrieved on the basis of sequence to determine where the insertion has occurred. Both in vivo and in vitro transposition methods are known. Both methods involve the use of a transposable element in combination with a transposase enzyme. When the transposable element or transposon is contacted with a nucleic acid fragment in the presence of the transposase, the transposable element will randomly insert into the nucleic acid fragment. The technique is useful for random mutageneis and for gene isolation, since the disrupted gene may be identified on the basis of the sequence of the transposable element. Kits for in vitro transposition are commercially available (see for example The Primer Island Transposition Kit, available from Perkin Elmer Applied Biosystems, Branchburg, N.J., based upon the yeast Ty1 element; The Genome Priming System, available from New England Biolabs, Beverly, Mass.; based upon the bacterial transposon Tn7; and the EZ::TN Transposon Insertion Systems, available from Epicentre Technologies, Madison, Wis., based upon the Tn5 bacterial transposable element). Transposon-mediated random insertion in the chromosome can be used for isolating mutants for any number of applications including enhanced production of any number of desired products including enzymes or other proteins, amino acids, or small organic molecules including alcohols.

The present invention has made use of this last method of pathway modulation to cause mutations in various essential genes to test whether there was any effect on the output of the carotenoid biosynthetic pathway. Transposon mutagenesis was used to create an E. coli mutant having a partial disruption in the yjeR gene. The precise sequence of the mutated gene is given as SEQ ID NO:63. This yjeR mutation (yjeR::Tn5 resulted in increased β-carotene production through an increase in plasmid copy number of the carotenoid producing plasmid (pPCB15 or pDCW108). The effect of mutation of this locus on plasmids is novel and could not have been predicted from known studies. Stacking the yjeR mutation (yjeR::Tn5) into the engineered E. coli strains that were made by chromosomal engineering of a non-endogenous promoter upstream of isoprenoid genes and chromosomally integrating non-endogenous isoprenoid pathway genes allowed further increases of β-carotene production.

The general methods described herein for pathway modulation are useful and enable the skilled person to practice the present invention. It will be appreciated that other, less traditional methods may be envisioned that will allow the practitioner to make the necessary modifications in the isoprenoid pathway. One such method involving chromosomal promoter replacement using a bacteriophage transduction system was used herein to good effect and is described below.

Optimization of Carotenoid Production in E. coli by Bacteriophage Transduction.

The present method combines promoter replacement via homologous recombination (in a recombination proficient host) with a bacteriophage transducing system. The method allows for the rapid insertion of strong promoters upstream of desired elements for increased gene expression. The method also facilitates the production of libraries to assess which combinations of expressable genetic elements will optimize production of the desired genetic end product (FIG. 12). In this way, genes not normally associated with a particular biosynthetic pathway may be identified which unexpectedly have significant effects on the production of the desired genetic end product.

Integration Cassettes

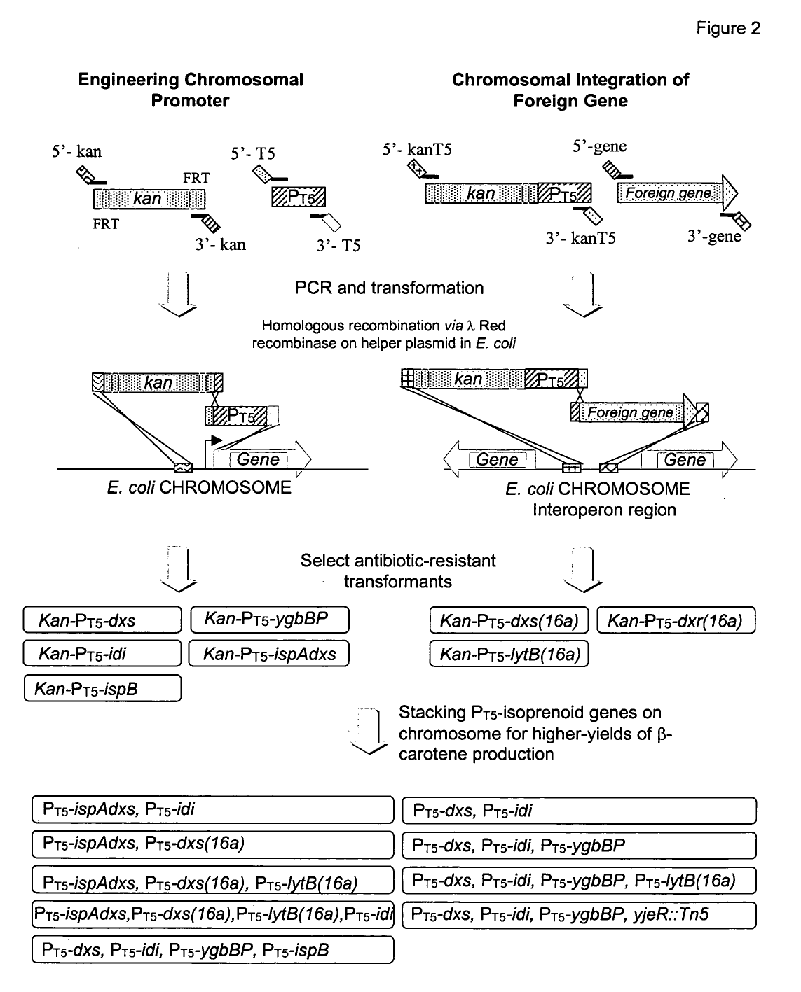

One aspect of the promoter replacement method is the use of an integration cassette. As used in the present invention, “integration cassettes” are the linear double-stranded DNA fragments chromosomally integrated by homologous recombination via the use of two PCR-generated fragments or one PCR-generated fragment as seen in FIG. 2. The integration cassette comprises a nucleic acid integration fragment that contains an expressible DNA fragment and a selectable marker bounded by specific recombinase sites responsive to a site-specific recombinase, and homology arms having homology to different portions of the host cell's chromosome. Typically, the integration cassette will have the general structure: 5′-RR1-RS-SM-RS-Y-RR2-3′ wherein

(i) RR1 is a first homology arm;

(ii) RS is a recombination site responsive to a site-specific recombinase;

(iii) SM is a DNA fragment encoding a selectable marker;

(iv) Y is a first expressible DNA fragment; and

(v) RR2 is a second homology arm.

Expressible DNA fragments of the invention are those that will be useful in genetically engineering biosynthetic pathways. For example, it may be useful to engineer a strong promoter in place of a native promoter in certain pathways. Virtually any promoter is suitable for the present invention including, but not limited to lac, ara, tet, trp, λP_{L}, λP_{R}, T7, tac, P_{T5}, and trc (useful for expression in Escherichia coli) as well as the amy, apr, npr promoters and various phage promoters useful for expression in Bacillus, for example.

Alternatively, different coding regions may be introduced downstream of existing native promoters. In this manner, new coding regions comprising a biosynthetic pathway may be introduced that either complete or enhance a pathway already in existence in the host cell. These coding regions may be genes which retain their native promoters or may be chimeric genes operably linked to an inducible or constitutive strong promoter for increased expression of the genes in the targeted biosynthetic pathway. Preferred in the present invention are the genes of the isoprenoid/carotenoid biosynthetic pathway, which include dxs, dxr, ygbP, ychB, ygbB, idi, ispA, lytB, gcpE, ispB, gps, crtE, crtY, crtI, crtB, crtX, and crtZ, as defined above and illustrated in FIG. 1. In the present invention, it is preferred if the expressible DNA fragment is a promoter or a coding region useful for modulation of a biosynthetic pathway. Exemplified in the present invention is the phage T5 strong promoter used for the modulation of the isoprenoid biosynthetic pathway in a recombinant proficient E. coli host. In some situations the expressible DNA fragment may be in antisense orientation where it is desired to down-regulate certain elements of the pathway.

Generally, the preferred length of the homology arms is about 10 to about 100 base pairs in length. Given the relatively short lengths of the homology arms used in the present invention for homologous recombination, one would expect that the level of acceptable mismatched sequences should be kept to an absolute minimum for efficient recombination, preferably using sequences which are identical to those targeted for homologous recombination. From 20 to 40 base pairs of homology, the efficiency of homologous recombination increases by four orders of magnitude (Yu et al. PNAS. 97:5978-5983. (2000)). Therefore, multiple mismatching within homology arms may decrease the efficiency of homologous recombination; however, one skilled in the art can easily ascertain the acceptable level of mismatching.

The present invention makes use of a selectable marker on one of the two recombination elements (integration cassettes). Selectable markers are known in the art including, but are not limited to antibiotic resistance markers such as ampicillin, kanamycin, and tetracycline resistance. Selectable markers may also include amino acid biosynthesis enzymes (for selection of auxotrophs normally requiring the exogenously supplied amino acid of interest) and enzymes which catalyze visible changes in appearance such as β-galactosidase in lac⁻ bacteria. As used herein, the markers are flanked by site-specific recombinase recognition sequences. After selection and construct verification, a site-specific recombinase is used to remove the marker. The steps of the present invention can then be repeated with additional in vivo chromosomal modifications. The integration cassette used to engineer the chromosomal modification includes a promoter and/or gene, and a selection marker flanked by site-specific recombinase sequences. Site-specific recombinases, such as the use of flippase (FLP) recombinase in the present invention, recognize specific recombination sequences (i.e. FRT sequences) and allow for the excision of the selectable marker. This aspect of the invention enables the repetitive use of the present process for multiple chromosomal modifications. The invention is not limited to the FLP-FRT recombinase system as several examples of site specific recombinases and their associated specific recognition sequences are know in the art. Examples of other suitable site-specific recombinases and their corresponding recognition sequences include: Cre-lox, R/RS, Gin/gix, Xer/dif, Int/att, a pSR1 system, a cer system, and a fim system.

Recombination Proficient Host Cells

The present invention makes use of a recombination proficient host cell that is able to mediate efficient homologous recombination between the integration cassettes and the host cell chromosome. Some organisms mediate homologous recombination very effectively (yeast for example) while others require genetic intervention. For example E. coli, a host generally considered as one which does not undergo efficient transformation via homologous recombination naturally, may be altered to make it a recombination proficient host. Transformation with a helper plasmid containing the λ-Red recombinase system increases the rate of homologous recombination several orders of magnitude (Murphy et al., Gene, 246:321-330 (2000); Murphy, K., J. Bacteriol., 180:2063-2071; Poteete and Fenton, J. Bacteriol., 182:2336-2340 (2000); Poteete, A., FEMS Microbiology Lett., 201:9-14 (2001); Datsenko and Wanner, supra; Yu et al., supra; Chaveroche et al., Nucleic Acids Research, 28:e97:1-6 (2000); U.S. Pat. No. 6,355,412; U.S. Pat. No. 6,509,156; and U.S. SN 60/434602). The λ-Red system can also be chromosomally integrated into the host. The λ-Red system contains three genes (exo, bet, and gam) which change the normally recombination deficient E. coli into a recombination proficient host.

Normally, E. coli efficiently degrades linear double stranded DNA via its RecBCD endonuclease, resulting in transformation efficiencies not useful for chromosomal engineering. The gam gene encodes for a protein that binds to the E.coli RecBCD complex, inhibiting endonuclease activity. The exo gene encodes for a λ-exonuclease which processively degrades the 5′ end strand of double stranded DNA and creates 3′ single stranded overhangs. The protein encoded by bet complexes with the λ-exonuclease and binds to the single-stranded DNA overhangs and promotes renaturation of complementary strands and is capable of mediating exchange reactions. The λ-Red recombinase system enables the use of homologous recombination as a tool for in vivo chromosomal engineering in hosts, such as E. coli, normally considered difficult to transform by homologous recombination. The λ-Red system works in other bacteria as well (Poteete, A., supra, 2001). Use of the λ-Red recombinase system should be applicable to other hosts generally used for industrial production. These additional hosts include, but are not limited to Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Paracoccus, Escherichia, Bacillus, Myxococcus, Salmonella, Yersinia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus. Preferred hosts are selected from the group consisting of Escherichia, Bacillus, and Methylomonas.

λ-Red Recombinase System

The λ-Red recombinase system used in the present invention is contained on a helper plasmid (pKD46) and is comprised of three essential genes, exo, bet, and gam (Datsenko and Wanner, supra). The exo gene encodes an λ-exonuclease, which processively degrades the 5′ end strand of double-stranded (ds) DNA and creates 3′ single-stranded overhangs. Bet encodes for a protein which complexes with the λ-exonuclease and binds to the single stranded DNA and promotes renaturation of complementary strands and is capable of mediating exchange reactions. Gam encodes for a protein that binds to the E.coli's RecBCD complex and blocks the complex's endonuclease activity.

The λ-Red system is used in the present invention because homologous recombination in E.coli occurs at a very low frequency and usually requires extensive regions of homology. The λ-Red system facilitates the ability to use short regions of homology (10-100 bp) flanking linear dsDNA fragments for homologous recombination. Additionally, the RecBCD complex normally expressed in E.coli prevents the use of linear dsDNA for transformation as the complex's exonuclease activity efficiently degrades linear dsDNA. Inhibition of the RecBCD complex's endonuclease activity by gam is essential for efficient homologous recombination using linear dsDNA fragments.

Combinatorial P1 Transduction System

Transduction is a phenomenon in which bacterial DNA is transferred from one bacterial cell (the donor) to another (the recipient) by a phage particle containing bacterial DNA. When a population of donor bacteria is infected with a phage, the events of the phage lytic cycle may be initiated. During lytic infection, the enzymes responsible for packaging viral DNA into the bacteriophage sometimes package host DNA. The resulting particle is called a transducing particle. Upon lysis of the cell, a mixture (“P1 lysate”) of transducing particles and normal virions are released. When this lysate is used to infect a population of recipient cells, most of the cells become infected with normal virus. However, a small proportion of the population receives transducing particles that inject the DNA they received from the previous host bacterium. This DNA can undergo genetic recombination with the DNA of the other host. Conventional P1 transduction can move only one genetic trait (i.e. gene) at a time (donor to receipient cell).

It will be appreciated that a number of host systems may be used for purposes of the present invention including, but not limited to those with known transducing phages such as Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Paracoccus, Escherichia, Bacillus, Myxococcus, Salmonella, Yersinia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus. Phages suitable for use in the present method may include, but are not limited to P1, P2, lambda, φ80, φ3538, T1, T4, P22, P22 derivatives, ES18, Felix “o”, P1-CmCs, Ffm, PY20, Mx4, Mx8, PBS-1, PMB-1, and PBT-1.

The present method provides a system for moving multiple genetic traits into a single E. coli host in a parallel combinatorial fashion using the bacteriophage P1 mixtures in combination with the site-specific recombinase system for removal of selection markers (FIG. 12). After P1 transduction with the P1 lysate mixture made from various donor cells, the transduced recipient cells are screened for antibiotic resistance and assayed for increased production of the desired genetic end product. After selection for the optimized transductants, the antibiotic resistance marker is removed by a site-specific recombinase. The selected transductants can be used again as a recipient cell in additional rounds of P1 transduction in order to engineer multiple chromosomal modifications, optimizing the production of the desired genetic end product. The present combinatorial P1 transduction method enables quick and easy chromosomal trait stacking for optimal production of the desired genetic end product.

Using the method described above, the promoters of the key isoprenoid genes that encode for rate-limiting enzymes involved in the isoprenoid pathway were engineered. Replacement of the endogenous promoters with a strong promoter (P_{T5}) resulted in increased β-carotene production.

An advantage of the present method of promoter replacement is that it allows for multiple chromosomal modifications within the host cell. The system is a means for moving multiple genetic traits into a single host cell using the bacteriophage P1 transduction in combination with a site-specific recombinase for removal of selection markers (FIGS. 2 and 12).

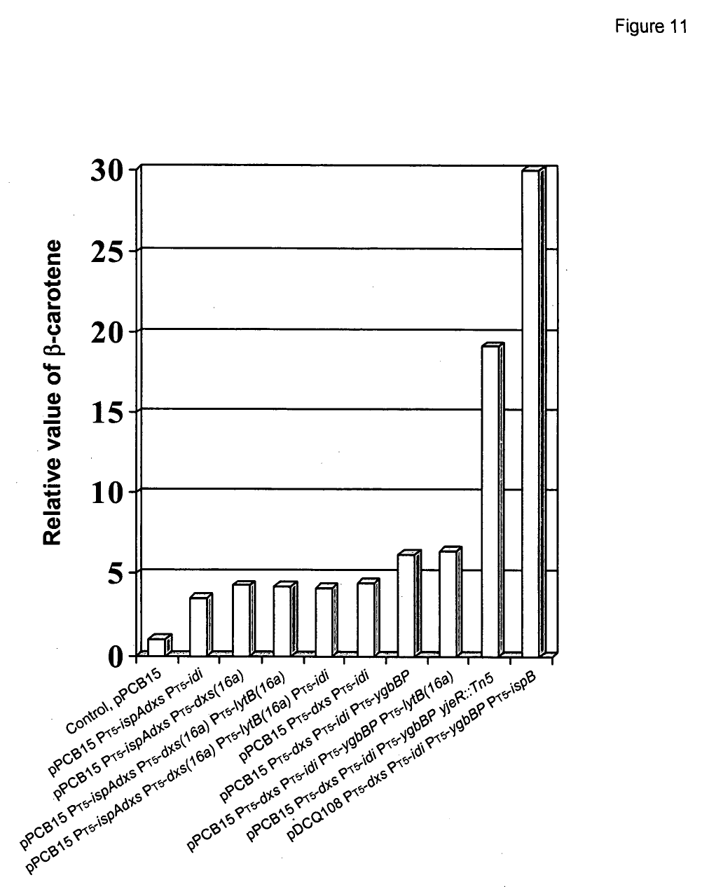

The present combinatorial P1 transduction method for promoter replacement enabled isolation and identification of the ispB gene and its effect on increasing the production of β-carotene when placed under the control of the strong promoter. The effect of ispB on increasing the production of β-carotene was an unexpected and non-obvious result. IspB (octaprenyl diphosphate synthase), which synthesizes the precursor of the side chain of the isoprenoid quinones, drains away the FPP substrate from the carotenoid biosynthetic pathway (FIG. 1). The mechanism of how overexpression of ispB gene under the control of phage T5 strong promoter increases the β-carotene production is not clear yet. However, the result suggests that IspB may increase the flux of the carotenoid biosynthetic pathway. Stacking the ispB gene under the control of a strong promoter into the chromosome of the engineered E. coli strains faciliated a further increase in β-carotene production (FIG. 11).

Measurement of the Carotenoid End Product

If the desired genetic end product is a colored product then transformants can be selected for on the basis of colored colonies, and the product can be quantitated by UV/vis spectrometry at the product's characteristic λₘₐₓ peaks. Alternative analytical methods can also be used including, but not limited to HPLC, CE, GC and GC-MS.

In the present invention, β-carotene was measured by UV/vis spectrometry at β-carotene's characteristic λₘₐₓ peaks at 425, 450 and 478 nm. The carotenoid was extracted by acetone from the cell pellet. The host strain included a reporter plasmid for the expression of genes involved in the synthesis of D-carotene. The reporter plasmid (pPCB15 or pDCQ108) carried the Pantoea stewartii crtEXYIB gene cluster. The gene cluster facilitated the production of β-carotene. Therefore, an increase of carbon flux through the isoprenoid upper pathway will result in an increase in the amount of β-carotene produced; resulting in colonies with more intense color on agar plates when compared to the strain that does not have T5 promoters engineered upstream of the isoprenoid genes. The amount of carotenoid produced was measured by HPLC analysis. Detection of β-carotene was measured by absorption at 450 nm at its respective retention time using HPLC under particular solvent conditions. Quantitative analysis was carried out by comparing the peak area for β-carotene to a known β-carotene standard.

### DESCRIPTION OF THE PREFERRED EMBODIMENTS

E. coli has been genetically modified to create several strains capable of enhanced production of β-carotene. One of the strains has been shown to produce up to 6 mg β-carotene per gram of dry cell weight.

Promoter replacement was accomplished using an easy one-step method of bacterial in vivo chromosomal engineering using two linear (PCR-generated) DNA fragments in order to increase carotenoid production in a host cell. The fragments were designed to contain short flanking regions of homology between the fragments and the target site on the host (E. coli) chromosome. The phage λ-Red recombinase system was expressed on a helper plasmid and under control of an arabinose-inducible promoter for controllable and efficient in vivo triple homologous recombination between the two PCR-generated DNA fragments and the host cell's chromosome. At least one of the two linear double stranded (ds) DNA fragments used during recombination was designed to contain a selective marker (kanamycin) flanked by site-specific recombinase sequences (FRT) (Example 1). The selectable marker permitted the identification and selection of the cells that had undergone the desired recombination event. The constructs of the selected recombinants were verified by sequence analysis. The selective marker was excised by a second helper plasmid (pCP20) containing the site-specific recombinase gene under the control of the PR promoter of λ phage (Examples 6-12 and 17).

A strong promoter (phage P_{T5}) was placed upstream of the E.coli target genes dxs, idi, ygbBygbP, ispB, ispAdxs (Example 1) via triple homologous recombination using two (PCR-generated) linear dsDNA fragments and the targeted chromosomal DNA (FIGS. 2). In each example, one of the two fragments contained a kanamycin resistance marker flanked by site-specific FRT recombinase sequences. Flanking the site-specific recombinase sequences were homology arms which contained short (approximately 10-50 bp) regions of homology. A first recombination region (homology arm #1) was linked to the 5′-end of the first fragment. A second recombination region (homology arm #2) was linked to the 3′-end of the first fragment. The second PCR generated linear dsDNA fragment contained the P_{T5} strong promoter. The third recombination region (homology arm #3) was linked to the 3′-end of the second fragment. The first recombination region (homology arm #1) had homology to an upstream portion of the native bacterial chromosomal promoter targeted for replacement. The second recombination region (homology arm #2 located on the 3′-end of the first fragment) had homology to the 5′-end portion of the second fragment. The third recombination region (homology arm #3) had homology to a downstream portion of the native bacterial chromosomal promoter targeted for replacement (FIG. 2).

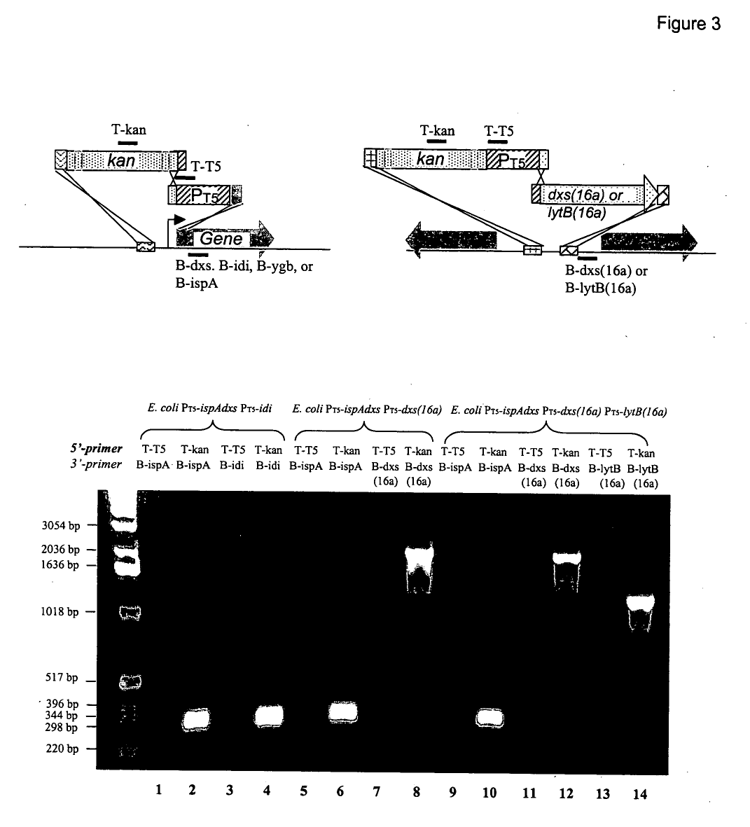

The recombination proficient E.coli host (containing the λ-Red recombination system on the helper plasmid pKD46) was transformed with the two PCR-generated fragments resulting in the chromosomal replacement of the targeted native promoter with the construct containing the kanamycin selectable marker of the first fragment and the P_{T5} strong promoter of the second fragment (Examples 1 and 6-12, FIG. 2). The promoter replacement resulted in the formation of an augmented E.coli chromosomal gene (either dxs, idi, ygbBygbP, ispB or ispAdxs genes), operably linked to the introduced non-native promoter. The bacterial host cells that had undergone the desired recombination event were selected according to the expression of the selectable marker and their ability to grow in selected media. The selected recombinants were then transformed with a second helper plasmid, pCP20 (Cherepanov and Wackernagel, supra), expressing the flippase (Flp) site-specific recombinase which excised the selectable marker (Examples 6-12). The constructs were confirmed via PCR fragment analysis (FIGS. 3-5). The recombinant bacterial host cell containing the augmented isoprenoid genes (dxs, idi, ygbBygbP, ispB or ispAdxs) and the carotenoid reporter plasmid (pPCB15) was then tested for increased production of β-carotene. Placement of one or more of the E. coli dxs, idi, ygbBygbP, ispB or ispAdxs genes (normally expressed at very low levels) under control of the strong P_{T5} promoter resulted in significant increases in β-carotene production (Examples 18-19, FIG. 11).

In another embodiment, the method was used to simultaneously add a foreign gene and promoter. The first of the two PCR-generated fragments was designed so that it contained the fusion product of a selectable marker (kanamycin) and promoter (P_{T5}) (Example 2, FIG. 2)). The second PCR-generated fragment contained the fusion product of a selectable marker (kan-P_{T5}) and the Methylomonas 16a dxs(16a) (SEQ ID NO:13), dxr(16a) (SEQ ID NO:17) or lytB(16a) (SEQ ID NO:15) genes (foreign to E. coli). Once again, homology arms were designed to allow for precise incorporation into the host bacterial chromosome. The desired recombinants were selected by methods previously described. The selectable marker was then removed by a site-specific recombinase as previously described. The recombinant constructs were confirmed by PCR fragment analysis. β-carotene production in the transformed E. coli reporter strain was measured as previously described. Cells containing the Methylomonas 16a dxs(16a) and/or lytB(16a) genes (homologous to the E. coli dxs and lytB genes) under the control of the P_{T5} promoter exhibited an increase in β-carotene production (FIG. 11). The present method was useful in the simultaneous addition of a foreign promoter and gene. Subsequent removal of the selectable marker is required so that the process can be repeated, if desired, to engineer bacterial biosynthetic pathways for increased production of the desired product.

In another embodiment, the bacterial host strain was engineered to contain multiple chromosomal modifications, including multiple promoter and gene additions or replacements so that the production efficiency of the desired final product is increased. In a preferred embodiment, the incorporated or augmented chromosomal genes encode for enzymes useful for the production of carotenoids.

In another preferred embodiment the constructs made by chromosomal engineering of non-endogenous promoters upstream of isoprenoid genes and chromosomally integrating non-endogenous isoprenoid pathway genes into the host chromosome are combined into a single strain. The phage T5 strong promoter (P_{T5})-ispAdxs P_{T5}-idi, P_{T5}-ispAdxs P_{T5}-dxs(16a), P_{T5}-ispAdxs P_{T5}-dxs(16a) P_{T5}-lytB(16a), P_{T5}-ispAdxs P_{T5}-dxs(16a) P_{T5}-lytB(16a) P_{T5}-idi, P_{T5}-dxs P_{T5}-idi, P_{T5}-dxs P_{T5}-idi P_{T5}-ygbBygbP, P_{T5}-dxs P_{T5}-idi P_{T5}-ygbBygbP P_{T5}-lytB(16a), P_{T5}-dxs P_{T5}-idi P_{T5}-ygbBygbP yjeR::Tn5, and P_{T5}-dxs P_{T5}-idi P_{T5}-ygbBygbP P_{T5}-ispB were constructed by combinatorial stacking. Stacking of these constructs in a combinatorial manner facilitated the development of engineered host strains capable of significantly increased carotenoid production.

In another embodiment, gene loci carrying transposon insertions that confer the ability to increase carotenoid production were engineered into the host chromosome. The E. coli yjeR gene carrying a Tn5 transposon insertion sequence (yjeR::Tn5; SEQ ID NO:63) was stacked in combination with P_{T5}-dxs, P_{T5}-idi and P_{T5}-ygbBygbP to create a strain producing 19-fold higher levels of β-carotene (ATCC PTA-4807).

In another embodiment, an E. coli reporter strain was constructed for assaying β-carotene production. Briefly, the reporter strain was created by cloning the gene cluster crtEXYIB from Pantoea stewartii into a reporter plasmid (pPCB15) that was subsequently used to transform the E.coli host (FIG. 7). The cluster contained many of the genes required for the synthesis of carotenoids, producing β-carotene in the transformed E. coli. It should be noted that the crtZ gene (β-carotene hydroxylase) was included in the gene cluster. However, since no promoter was present to express the crtZ gene (organized in opposite orientation and adjacent to crtB gene), no zeaxanthin was produced. The zeaxanthin glucosyl transferase enzyme (encoded by the crtX gene located within the gene cluster) had no substrate for its reaction. Increases in β-carotene production were reported as increases relative to the control strain production (FIG. 11).

In another embodiment, a new reporter plasmid was created. Reporter plasmid pPCB15, used for many of the experiments, is considered a low copy number plasmid. A new medium-copy number reporter plasmid was generated, (pDCQ108) that also contained the Pantoea stewartii crtEXYIB gene cluster (Example 19). Plasmid pDCQ108 was then used as the reporter plasmid in E.coli P_{T5} dxs P_{T5}-idi P_{T5}-ygbBygbP P_{T5}-ispB leading to an approximately 30-fold increase in β-carotene production when compared to the control strain (FIG. 11; Examples 20 and 21; Table 9)).

It has been speculated that the limits for carotenoid production in non-carotenogenic host such as E. coli had been reached at the level of around 1.5 mg/g cell dry weight (1,500 ppm) due to overload of the membranes and blocking of membrane functionality (Albrecht et al., supra). The present method has solved the stated problem by making modifications on the E. coli chromosome that resulted in increased β-carotene production of up to 6 mg per gram dry cell weight (6,000 ppm), an increase of 30-fold over initial levels with no lethal effect. The bacterial production of 6,000 ppm carotenoids is much higher than the maximum accepted limit (1,600 ppm) for carotneoid production in bacteria.

One of skill in the art will recognize that the present method can be applied to a variety of hosts in addition to E. coli. Use of the present method in other hosts is supported by the fact that: 1) the isoprenoid pathway is common in bacteria, 2) the λ-Red system has been reported to work in a variety of hosts, and 3) phage transduction is known to occur in many hosts.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### General Methods

Standard recombinant DNA and molecular cloning techniques used in the Examples are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987).

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989). All reagents, restriction enzymes and materials used for the growth and maintenance of bacterial cells were obtained from Aldrich Chemicals (Milwaukee, Wis.), DIFCO Laboratories (Detroit, Mich.), GIBCO/BRL (Gaithersburg, Md.), or Sigma Chemical Company (St. Louis, Mo.) unless otherwise specified.

Manipulations of genetic sequences were accomplished using the suite of programs available from the Genetics Computer Group Inc. (Wisconsin Package Version 9.0, Genetics Computer Group (GCG), Madison, Wis.). Where the GCG program “Pileup” was used the gap creation default value of 12, and the gap extension default value of 4 were used. Where the CGC “Gap” or “Besffit” programs were used the default gap creation penalty of 50 and the default gap extension penalty of 3 were used. Multiple alignments were created using the FASTA program incorporating the Smith-Waterman algorithm (W. R. Pearson, Comput. Methods Genome Res., [Proc. Int. Symp.] (1994), Meeting Date 1992, 111-120. Editor(s): Suhai, Sandor. Publisher: Plenum, New York, N.Y.). In any case where program parameters were not prompted for, in these or any other programs, default values were used.

The meaning of abbreviations is as follows: “h” means hour(s), “min” means minute(s), “sec” means second(s), “d” means day(s), “μL” means microliter(s), “mL” means milliliter(s), “L” means liter(s), and “rpm” means revolutions per minute.

### Example 1

### Construction of E. coli Strains with the phage P_{T5} Promoter Chromosomally-integrated Upstream of the Isoprenoid Genes (Promoter Replacement)

The native promoters of the E. coli isoprenoid genes dxs, idi, ygbBygbP, ispB, and ispAdxs, (FIG. 1) were replaced with the (P_{T5}) promoter using two PCR-fragments chromosomal integration method as described in FIG. 2. The method for replacement is based on homologous recombination via the λ-Red recombinase encoded on a helper plasmid. Recombination occurs between the E. coli chromosome and two PCR fragments that contain 20-50 bp homology patches at both ends of PCR fragments (FIG. 2). For integration of the P_{T5} promoter upstream of these genes, a two PCR fragment method was employed. In this method, the two linear fragments included a DNA fragment (1489 bp) containing a kanamycin selectable marker (kan) flanked by site-specific recombinase target sequences (FRT) and a DNA fragment (154 bp) containing a phage T5 promoter (P_{T5}) comprising the −10 and −35 consensus promoter sequences, lac operator (lacO), and a ribosomal binding site (rbs).

By using the two PCR fragment method, the kanamycin selectable marker and P_{T5} promoter (kan-P_{T5}) were integrated upstream of the dxs, idi, ygbBP, ispB, and ispAdxs genes, yielding kan-P_{T5}-dxs, kan-P_{T5}-idi, kan-P_{T5}-ygbBP, kan-P_{T5}-ispB, and kan-P_{T5}-ispAdxs. The linear DNA fragment (1489 bp) containing a kanamycin selectable marker was synthesized by PCR from plasmid pKD4 (Datsenko and Wanner, supra) with primer pairs as follows in Table 3.

TABLE 3

Primers for Amplification of the Kanamycin
Selectable Marker

| | | SEQ ID |
|---|---|---|
| Primer Name | Primer Sequence | NO: |
| 5′-kan(dxs) | TGGAAGCGCTAGCGGACTACATCATCCA | 21 |
| | GCGTAATAAATAACGTCTTGAGCGATTGT | |
| | GTAG¹ | |
| 5′-kan(idi) | TCTGATGCGCAAGCTGAAGAAAAATGAGC | 22 |
| | ATGGAGAATAATATGACGTCTTGAGCGAT | |
| | TGTGTAG¹ | |
| 5′- | GACGCGTCGAAGCGCGCACAGTCTGCGG | 23 |
| kan(ygbBP) | GGCAAAACAATCGATAACGTCTTGAGCGA | |
| | TTGTGTAG¹ | |
| 5′- | ACCATGACGGGGCGAAAAATATTGAGAG | 24 |
| kan(ispAdxs) | TCAGACATTCATGTGTAGGCTGGAGCTGC | |
| | TTC¹ | |
| 3′-kan | GAAGACGAAAGGGCCTCGTGATACGCCT | 25 |
| | ATTTTTATAGGTTATATGAATATCCTCCTT | |
| | AGTTCC² | |

¹The underlined sequences illustrate each respective homology arm chosen to match sequences in the upstream region of the chromosomal integration site, while the remainder is the priming sequence
²The underlined sequences illustrate homology arm chosen to match sequences in the 5′-end region of the T5 promoter DNA fragment

The second linear DNA fragment (154 bp) containing the P_{T5} promoter was synthesized by PCR from pQE30 (QIAGEN, Inc. Valencia, Calif.) with primer pairs as follows in Table 4.

TABLE 4
Primers for Amplification of the P_{T5} Promoter

| | | SEQ ID |
|---|---|---|
| Primer Name | Primer Sequence | NO: |
| 5′-T5 | CTAAGGAGGATATTCATATAACCTATAAAA | 26 |
| | ATAGGCGTATCACGAGGCCC¹ | |
| 3′-T5(dxs) | GGAGTCGACCAGTGCCAGGGTCGGGTATT | 27 |
| | TGGCAATATCAAAACTCATAGTTAATTTCTC | |
| | CTCTTTAATG² | |
| 3′-T5(idi) | TGGGAACTCCCTGTGCATTCAATAAAATGA | 28 |
| | CGTGTTCCGTTTGCATAGTTAATTTCTCCT | |
| | CTTTAATG² | |
| 3′- | CGGCCGCCGGAACCACGGCGCAAACATC | 29 |
| T5(ygbBP) | CAAATGAGTGGTTGCCATAGTTAATTTCTC | |
| | CTCTTTAATG² | |
| 3′- | CCTGCTTAACGCAGGCTTCGAGTTGCTGC | 30 |
| T5(ispAdxs) | GGAAAGTCCATAGTTAATTTCTCCTCTTTA | |
| | ATG² | |

¹The underlined sequences illustrate homology arm chosen to match sequences in the 3′-end region of the kanamycin DNA fragment
²The underlined sequences illustrate each respective homology arm chosen to match sequences in the downstream region of the chromosomal integration site

The linear DNA fragment (1,647 bp) containing fused kanamycin selectable marker-phage T5 promoter is synthesized by PCR from pSUH5 with primer pairs as follows in Table 5. The pSUH5 plasmid (FIG. 6; SEQ ID NO:66) was constructed by cloning a phage T5 promoter (P_{T5}) region (SEQ ID NO:33) into the NdeI restriction endonuclease site of pKD4 (Datsenko and Wanner, supra).

TABLE 5

Primers for Amplification of the Fused
Kanamycin Selectable Marker-Phage
P_{T5} Promoter

| | | SEQ ID |
|---|---|---|
| Primer Name | Primer Sequence | NO: |
| 5′- | ACCATAAACCCTAAGTTGCCTTTGTTCACA | 31 |
| kanT5(ispB) | GTAAGGTAATCGGGGCGTCTTGAGCGATT | |
| | GTGTAG¹ | |
| 3′- | CGCCATATCTTGCGCGGTTAACTCATTGA | 32 |
| kanT5(ispB) | TTTTTTCTAAATTCATAGTTAATTTCTCCTC | |
| | TTTAATG² | |

¹The underlined sequences illustrate each respective homology arm chosen to match sequences in the upstream region of the chromosomal integration site.
²The underlined sequences illustrate each respective homology arm chosen to match sequences in the downstream region of the chromosomal integration site.

Standard PCR conditions were used to amplify the linear DNA fragments with AmpliTaq Gold® polymerase (Applied Biosystems, Foster City, Calif.) as follows:

| PCR reaction: | PCR reaction mixture: |
|---|---|
| Step1 94° C. 3 min | 0.5 μL plasmid DNA |
| Step2 93° C. 30 sec | 12 5 μL 10X PCR buffer |
| Step3 55° C. 1 min | 1 μL dNTP mixture (10 mM) |
| Step4 72° C. 3 min | 1 μL 5′-primer (20 μM) |
| Step5 Go To Step2, 30 cycles | 1 μL 3′-primer (20 μM) |
| Step6 72° C. 5 min | 0.5 μL AmpliTaq Gold ® |
| polymerase | 41 μL sterilized dH₂O |

After completing the PCR reactions, 50 μL of each PCR reaction mixture was run on a 1% agarose gel and the PCR products were purified using the QIAquick Gel Extraction Kit™ as per the manufacturer's instructions (Cat. #28704, QIAGEN Inc., Valencia, Calif.). The PCR products were eluted with 10 μL of distilled water. The DNA Clean & Concentrator™ kit (Zymo Research, Orange, Calif.) was used to further purify the PCR product fragments as per the manufacturer's instructions. The PCR products were eluted with 6-8 μL of distilled water to a concentration of 0.5-1.0 μg/μL.

The E. coli MC1061 strain, carrying the λ-Red recombinase expression plasmid pKD46 (amp^{R}) (SEQ ID NO:65) was used as a host strain for the chromosomal integration of the PCR fragments. The strain was constructed by transformation of E. coli strain MC1061 with the λ-Red recombinase expression plasmid, pKD46 (amp^{R}). Transformants were selected on 100 μg/mL ampicillin LB plates at 30° C.

For transformation, electroporation was performed using 1-5 μg of the purified PCR products carrying the kanamycin marker and P_{T5} promoter. Approximately one-half of the cells transformed were spread on LB plates containing 25 μg/mL kanamycin in order to select antibiotic-resistant transformants. After incubating the plate at 37° C. overnight, antibiotic-resistance transformants were selected as follows: 10 colonies of kan-P_{T5}-dxs, 12 colonies of kan-P_{T5}-idi, 10 colonies of kan-P_{T5}-ygbBP, 3 colonies of kan-P_{T5}-ispB, and 19 colonies of kan-P_{T5}-ispA.

PCR analysis was used to confirm the integration of both the kanamycin selectable marker and the P_{T5} promoter in the correct location on the E. coli chromosome. For PCR, a colony was resuspended in 50 μL of PCR reaction mixture containing 200 μM dNTPs, 2.5 U AmpliTaq™ (Applied Biosytems), and 0.4 μM of specific primer pairs. Test primers were chosen to match sequences of the regions located in the kanamycin (5′-primer) and the early coding-region of each isoprenoid gene (3′-primer) (FIG. 3). Sequences of these primers are listed in Tables 3, 4, and 5 above and the PCR reaction was performed as described above. The resultant E. coli strains carrying each kan-P_{T5}-isoprenoid gene fusion on the chromosome were used for stacking multiple kan-P_{T5}-isoprenoid gene fusions on the chromosome to construct E. coli strain for increasing β-carotene production as described in Examples 6-12 and 17.

### Example 2

### Construction of E. coli Strains with Methylomonas 16A dxs(16A), dxr(16A) and lytB(16A) Genes Chromosomally-integrated

Methylomonas 16a (ATCC PTA-2402) isoprenoid genes dxs, dxr and lytB (WO 02/20733 A2), with dxs (denoted as “dxs(16a)” and described as SEQ ID NO:13), dxr (denoted as “dxr(16a)” and described as SEQ ID NO:17), and lytB (denoted as “lytB(16a)” and described by SEQ ID NO:15), and the fused kan-P_{T5} promoter were co-integrated into the inter-operon regions located at 30.9, 78.6 and 18.1 min, respectively, of the E. coli chromosome using the two PCR-fragments chromosomal integration method as described in FIG. 2. The principle for chromosomal integration of foreign gene is same as described in Example 1.

The linear DNA fragment (1,647 bp) containing fused kanamycin selectable marker-P_{T5} promoter was synthesized by PCR from pSUH5 with primer pairs as follows in Table 6. The pSUH5 plasmid (FIG. 6) was constructed by cloning a P_{T5} promoter region (SEQ ID NO:33) into the NdeI restriction endonuclease site of pKD4 (Datsenko and Wanner, supra).

TABLE 6

Primers for Amplification of the Fused
Kanamycin Selectable Marker-P_{T5} Promoter

| | | SEQ ID |
|---|---|---|
| Primer Name | Primer Sequence | NO: |
| 5′- | CACTAACGCCCGCACATTGCTGCGGGC | 34 |
| kanT5(dxs16a) | TTTTTGATTCATTTCGCACGTCTTGAGC | |
| | GATTGTGTAG¹ | |
| 5′- | TAAAGGGCTAAGAGTAGTGTGCTCTTA | 35 |
| kanT5(dxr16a) | GCCCTTAATTACGTTTCCCGTCTTGAGC | |
| | GATTGTGTAG¹ | |
| 5′- | CTACAACTGGCGAGATGCATAGCGAGT | 36 |
| kanT5(lytB16a) | ATAATTTGTATTTTGCGTCGTCTTGAGC | |
| | GATTGTGTAG¹ | |
| 3′- | AGTAGAGGGAAGTCTTTGGAAAGAGCC | 37 |
| kanT5(dxs16a) | ATAGTTAATTTCTCCTCTTTAATG² | |
| 3′- | ACGGTGCCGCCGCAATGATGCTGTCCA | 38 |
| kanT5(dxr16a) | CCAGTTAATTTCTCCTCTTTAATG² | |
| 3′- | CCACGGGGGTTTGCGAGTACGATTTGC | 39 |
| kanT5(lytB16a) | ATAGTTAATTTCTCCTCTTTAATG² | |

¹The underlined sequences illustrate each respective homology arm chosen to match sequences in the upstream region of the chromosomal integration site, while the remainder is the priming sequence
²The underlined sequences illustrate homology arm chosen to match sequences in the 5′-end region of the foreign gene DNA fragment

The linear DNA fragment containing Methylomonas 16a dxs, dxr or lytB gene was synthesized by PCR from Methylomonas 16a (ATCC PTA-2402) genomic DNA with primer pairs as follows in Table 7.

TABLE 7
Primers for Amplification of the Foreign Gene

| | | SEQ ID |
|---|---|---|
| Primer Name | Primer Sequence | NO: |
| 5′-dxs16a | ACAGAATTCATTAAAGAGGAGAAATTAACT | 40 |
| | ATGGCTCTTTCCAAAGAC TTCCCTC¹ | |
| 5′-dxr16a | ACAGAATTCATTAAAGAGGAGAAATTAACT | 41 |
| | GGTGGACAGCATCATTGCGGCGGCA¹ | |
| 5′-lytB16a | ACAGAATTCATTAAAGAGGAGAAATTAACT | 42 |
| | ATGCAAATCGTACTCGCAAACCCCC¹ | |
| 3′-dxs16a | AGGAGCGAAGTGATTATCAGTATGCTGTTC | 43 |
| | ATATAGCCTCGAATTATCAAGCGCAAAACT | |
| | GTTCGATG² | |
| 3′-dxr16a | GGCATTTTCACTCTGGCAATGCGCATAAAC | 44 |
| | GCTTTCAAAGTCCTGTTAAGCTACCAAGGT | |
| | CTTGATG² | |
| 3′-lytB16a | AGTGGCGGACGGGCAAACAAGGGTAACAT | 45 |
| | AGGATCAATGAGGGTTATTGATCACGCTTG | |
| | CATATGTTT² | |

¹The underlined sequences illustrate homology arm chosen to match sequences in the 3′-end region of the fused kanamycin-phage P_{T5} promoter DNA fragment
²The underlined sequences illustrate each respective homology arm chosen to match sequences in the downstream region of the chromosomal integration site

The PCR reaction, purification and electro-transformation were performed as described in Example 1. Kanamycin-resistance transformants were selected including 7 colonies of E. coli kan-P_{T5}-dxs(16a), 3 colonies of E. coli kan-P_{T5}-dxr(16a) and 12 colonies of E. coli kan-P_{T5}-lytB(16a). Among these, the colonies that have a correct integration of kan-P_{T5}-dxs(16a), kan-P_{T5}-dxr(16a) or kan-P_{T5}-lytB(16a) into the target site of E. coli chromosome was selected by PCR analysis (FIG. 3, 4, and 5).

### Example 3

### Cloning of β-Carotene Production Genes from Pantoea stewartii

Primers were designed using the sequence from Erwinia uredovora to amplify a fragment by PCR containing the crt genes. These sequences included 5′-3′:

| | |
|---|---|
| ATGACGGTCTGCGCAAAAAAACACG | SEQ ID NO:19 |
| GAGAAATTATGTTGTGGATTTGGAATGC | SEQ ID NO:20 |

Chromosomal DNA was purified from Pantoea stewartii (ATCC no. 8199) and Pfu Turbo polymerase (Stratagene, La Jolla, Calif.) was used in a PCR amplification reaction under the following conditions: 94° C., 5 min; 94° C. (1 min)-60° C. (1 min)-72° C. (10 min) for 25 cycles, and 72° C. for 10 min. A single product of approximately 6.5 kb was observed following gel electrophoresis. Taq polymerase (Perkin Elmer, Foster City, Calif.) was used in a ten minute 72° C. reaction to add additional 3′ adenosine nucleotides to the fragment for TOPO cloning into pCR4-TOPO (Invitrogen, Carlsbad, Calif.) to create the plasmid pPCB13. Following transformation to E. coli DH5α (Life Technologies, Rockville, Md.) by electroporation, several colonies appeared to be bright yellow in color indicating that they were producing a carotenoid compound. Following plasmid isolation as instructed by the manufacturer using the Qiagen (Valencia, Calif.) miniprep kit, the plasmid containing the 6.5 kb amplified fragment was transposed with pGPS1.1 using the GPS-1 Genome Priming System kit (New England Biolabs, Inc., Beverly, Mass.). A number of these transposed plasmids were sequenced from each end of the transposon. Sequence was generated on an ABI Automatic sequencer using dye terminator technology (U.S. Pat. No. 5,366,860; EP 272007) using transposon specific primers. Sequence assembly was performed with the Sequencher program (Gene Codes Corp., Ann Arbor Mich.).

### Example 4

### Identification and Characterization of Bacterial Genes

Genes encoding crtE, X, Y, I, B, and Z were identified by conducting BLAST (Basic Local Alignment Search Tool; Altschul, S. F., et al., J. Mol. Biol. 215:403-410 (1993)) searches for similarity to sequences contained in the BLAST “nr” database (comprising all non-redundant GenBank® CDS translations, sequences derived from the 3-dimensional structure Brookhaven Protein Data Bank, the SWISS-PROT protein sequence database, EMBL, and DDBJ databases). The sequences obtained in Example 3 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish, W. and States, D., Nature Genetics, 3:266-272 (1993)) provided by the NCBI.

All comparisons were done using either the BLASTNnr or BLASTXnr algorithm. The results of the BLAST comparison are given in Table 7 which summarize the sequences to which they have the most similarity. Table 7 displays data based on the BLASTXnr algorithm with values reported in expect values. The Expect value estimates the statistical significance of the match, specifying the number of matches, with a given score, that are expected in a search of a database of this size absolutely by chance.

TABLE 8

| | | | SEQ ID | SEQ ID | | | | |
|---|---|---|---|---|---|---|---|---|
| ORF | Gene | | No. | No. | % | % | | |
| Name | Name | Similarity Identified | base | Peptide | Identity^{a} | Similarity^{b} | E-value^{c} | Citation |
| 1 | crtE | Geranylgeranyl pryophosphate synthetase (or | 1 | 2 | 83 | 88 | e−137 | Misawa et al., J. |
| | | GGPP synthetase, or farnesyltranstransferase) | | | | | | Bacteriol. 172 |
| | | EC 2.5.1.29 | | | | | | (12), 6704-6712 |
| | | | | | | | | (1990) |
| | | gi\|117509\|sp\|P21684\|CRTE₋ PANAN | | | | | | |
| | | GERANYLGERANYL PYROPHOSPHATE | | | | | | |
| | | SYNTHETASE (GGPP SYNTHETASE) | | | | | | |
| | | (FARNESYLTRANSTRANSFERASE) | | | | | | |
| 2 | crtX- | Zeaxanthin glucosyl transferase EC 2.4.1.- | 3 | 4 | 75 | 79 | 0.0 | Lin et al., Mol. |
| | | | | | | | | Gen. Genet. |
| | | gi\|1073294\|pir∥S52583 crtX protein - | | | | | | |
| | | Erwinia herbicola | | | | | | 245 (4), 417-423 |
| | | | | | | | | (1994) |
| 3 | crtY | Lycopene cyclase | 5 | 6 | 83 | 91 | 0.0 | Lin et al., Mol. |
| | | | | | | | | Gen. Genet. |
| | | gi\|1073295\|pir∥S52585 lycopene cyclase - | | | | | | |
| | | Erwinia herbicola | | | | | | 245 (4), 417-423 |
| | | | | | | | | (1994) |
| 4 | crtI | Phytoene desaturaseEC 1.3.-.- | 7 | 8 | 89 | 91 | 0.0 | Lin et al., Mol. |
| | | | | | | | | Gen. Genet. |
| | | gi\|1073299\|pir∥S52586 phytoene | | | | | | |
| | | dehydrogenase (EC 1.3.-.-) - Erwinia herbicola | | | | | | 245 (4), 417-423 |
| | | | | | | | | (1994) |
| 5 | crtB | Phytoene synthaseEC2.5.1.- | 9 | 10 | 88 | 92 | e−150 | Lin et al., Mol. |
| | | | | | | | | Gen. Genet. |
| | | gi\|1073300\|pir∥S52587 prephytoene | | | | | | |
| | | pyrophosphate synthase - Erwinia herbicola | | | | | | 245 (4), 417-423 |
| | | | | | | | | (1994) |
| 6 | crtZ | Beta-carotene hydroxylase | 11 | 12 | 88 | 91 | 3e−88 | Misawa et al., J. |
| | | | | | | | | Bacteriol. 172 |
| | | gi\|117526\|sp\|P21688\|CRTZ₋ PANAN BETA- | | | | | | |
| | | CAROTENE HYDROXYLASE | | | | | | (12), 6704-6712 |
| | | | | | | | | (1990) |

^{a}% Identity is defined as percentage of amino acids that are identical between the two proteins.
^{b}% Similarity is defined as percentage of amino acids that are identical or conserved between the two proteins.
^{c}Expect value. The Expect value estimates the statistical significance of the match, specifying the number of matches, with a given score, that are expected in a search of a database of this size absolutely by chance.

### Example 5

### Analysis of Gene Function by Transposon Mutagenesis

Several plasmids carrying transposons which were inserted into each coding region including crtE, crtX, crtY, crtI, crtB, and crtZ were chosen using sequence data generated in Example 3. These plasmid variants were transformed to E. coli MG1655 and grown in 100 mL Luria-Bertani broth in the presence of 100 μg/mL ampicillin. Cultures were grown for 18 hr at 26° C., and the cells were harvested by centrifugation. Carotenoids were extracted from the cell pellets using 10 mL of acetone. The acetone was dried under nitrogen and the carotenoids were resuspended in 1 mL of methanol for HPLC analysis. A Beckman System Gold® HPLC with Beckman Gold Nouveau Software (Columbia, Md.) was used for the study. The crude extraction (0.1 mL) was loaded onto a 125×4 mm RP8 (5 μm particles) column with corresponding guard column (Hewlett-Packard, San Fernando, Calif.). The flow rate was 1 mL/min, while the solvent program used was: 0-11.5 min 40% water/60% methanol; 11.5-20 min 100% methanol; 20-30 min 40% water/60% methanol. The spectrum data were collected by the Beckman photodiode array detector (model 168).

In the clone with wild type crtEXYIBZ, the carotenoid was found to have a retention time of 15.8 min and an absorption spectra of 450 nm, 475 nm. This was the same value observed in comparison to the β-carotene standard. This suggested that crtZ gene organized in the opposite orientation was not expressed in this construct. The transposon insertion in crtZ had no effect as expected (data not shown).

HPLC spectral analysis also revealed that a clone with transposon insertion in crtX also produced β-carotene. This is consistent with the proposed function of crtX encoding a zeaxanthin glucosyl transferase enzyme at a later step of the carotenoid pathway following synthesis of β-carotene.

The transposon insertion in crtY did not produce β-carotene. The carotenoid's elution time (15.2 min) and absorption spectra (443 nm, 469 nm, 500 nm) agree with those of the lycopene standard. Accumulation of lycopene in the crtY mutant confirmed the role crtY as a lycopene cyclase encoding gene.

The crtI extraction, when monitored at 286 nm, had a peak with retention time of 16.3 min and with absorption spectra of 276 nm, 286 nm, 297 nm, which agrees with the reported spectrum for phytoene. Detection of phytoene in the crtI mutant confirmed the function of the crtI gene as one encoding a phytoene dehydrogenase enzyme.

The acetone extracted from the crtE mutant or crtB mutant was clear. Loss of pigmented carotenoids in these mutants indicated that both the crtE gene and crtB genes are essential for carotenoid synthesis. No carotenoid was observed in either mutant, which is consistent with the proposed function of crtB encoding a prephytoene pyrophosphate synthase and crtE encoding a geranylgeranyl pyrophosphate synthetase.

Both enzymes are required for β-carotene synthesis. Results of the transposon mutagenesis experiments are shown below in Table 9. The site of transposon insertion into the gene cluster crtEXYIB is recorded, along with the color of the E. coli colonies observed on LB plates, the identity of the carotenoid compound (as determined by HPLC spectral analysis), and the experimentally assigned function of each gene.

TABLE 9
Transposon Insertion Analysis of Carotenoid Gene Function

| | | Carotenoid | |
|---|---|---|---|
| Transposon | Colony | observed by | |
| insertion site | color | HPLC | Assigned gene function |
| Wild Type (with | Yellow | β-carotene | |
| no transposon | | | |
| insertion) | | | |
| crtE | White | None | Geranylgeranyl pyrophosphate |
| | | | synthetase |
| crtB | White | None | Prephytoene pyrophosphate |
| | | | synthase |
| crtI | White | Phytoene | Phytoene dehydrogenase |
| crtY | Pink | Lycopene | Lycopene cyclase |
| crtZ | Yellow | β-carotene | β-carotene hydroxylase |
| crtX | Yellow | β-carotene | Zeaxanthin glucosyl |
| | | | transferase |

### Example 6

### Construction of E. coli P_{T5}-ispAdxs P_{T5}-idi Strain for Increased β-Carotene Production

In order to characterize the effect of the chromosomal integration of the P_{T5} promoter in the front of the isoprenoid genes on β-carotene production, a strain (E. coli P_{T5}-ispAdxs P_{T5}-idi) containing a chromosomally integrated P_{T5} promoter upstream from ispAdxs and idi genes and capable of producing β-carotene was constructed.

First, P1 lysate of the E. coli kan-P_{T5}-ispAdxs strain was prepared by infecting a growing culture of bacteria with the P1 phage and allowing the cells to lyse. For P1 infection, E. coli kan-P_{T5}-ispAdxs strain was inoculated in 4 mL LB medium with 25 μg/mL kanamycin, grown at 37° C. overnight, and then sub-cultured with 1:100 dilution of an overnight culture in 10 mL LB medium containing 5 mM CaCl₂. After 20-30 min of growth at 37° C., 10⁷ P1ᵥᵢᵣ phages were added. The cell-phage mixture was aerated for 2-3 h at 37° C. until lysed, several drops of chloroform were added and the mixture vortexed for 30 sec and incubated for an additional 30 min at room temp. The mixture was then centrifuged for 10 min at 4500 rpm, and the supernatant transferred into a new tube to which several drops of chloroform were added.

Second, P1 lysate made on E. coli kan-P_{T5} ispAdxs strain was transduced into the recipient strain, E. coli MG1655 containing a β-carotene biosynthesis expression plasmid pPCB15 (cam^{R}) (FIG. 6). The plasmid pPCB15 (cam^{R}) encodes the carotenoid biosynthesis gene cluster (crtEXYIB) from Pantoea Stewartii (ATCC no. 8199). The pPCB15 plasmid was constructed from ligation of SmaI digested pSU18 (Bartolome et al., Gene, 102:75-78 (1991)) vector with a blunt-ended PmeI/NotI fragment carrying crtEXYIB from pPCB13 (Example 3). The E. coli MG1655 pPCB15 recipient cells were grown to mid-log phase (1-2×10⁸ cells/ml) in 4 mL LB medium with 25 μg/mL chloramphenicol at 37° C. Cells were spun down for 10 min at 4500 rpm and resuspended in 2 mL of 10 mM MgSO₄ and 5 mM CaCl₂. Recipient cells (100 μL) were mixed with 1 μL, 10 μL, or 100 μL of P1 lysate stock (10⁷ pfu/μL) made from the E. coli kan-P_{T5}-ispAdxs strain and incubated at 30° C. for 30 min. The recipient cell-lysate mixture was spun down at 6500 rpm for 30 sec, resuspended in 100 μL of LB medium with 10 mM of sodium citrate, and incubated at 37° C. for 1 h. Cells were plated on LB plates containing both 25 μg/mL kanamycin and 25 μg/mL chloramphenicol in order to select for antibiotic-resistant transductants and incubated at 37° C. for 1 or 2 days. Six kanamycin-resistance transductants were selected.

To eliminate kanamycin selectable marker from the chromosome, a FLP recombinase expression plasmid pCP20 (amp^{R}) (ATCC PTA-4455) (Cherepanov and Wackernagel, supra), which has a temperature-sensitive replication of origin, was transiently transformed into one of the kanamycin-resistant transductants by electroporation. Cells were spread onto LB agar containing 100 μg/mL ampicillin and 25 μg/mL chloramphenicol LB plates, and grown at 30° C. for 1 day. Colonies were picked and streaked on 25 μg/mL chloramphenicol LB plates without ampicillin antibiotics and incubated at 43° C. overnight. Plasmid pCP20 has a temperature sensitive origin of replication and was cured from the host cells by culturing cells at 43° C. The colonies were tested for ampicillin and kanamycin sensitivity to test loss of pCP20 and kanamycin selectable marker by streaking colonies on 100 μg/mL ampicillin LB plate or 25 μg/mL kanamycin LB plate. In this manner the E. coli P_{T5}-ispAdxs strain was constructed

In order to further stack kan-P_{T5}-idi on chromosome of E. coli P_{T5}-ispAdxs, P1 lysate made on E. coli kan-P_{T5}-idi strain was transduced into the recipient strain, E. coli P_{T5}-ispAdxs, as described above. Approximately 85 transductants were selected. After transduction, the kanamycin selectable marker was eliminated from the chromosome as described above, yielding E. coli P_{T5}-ispAdxs P_{T5}-idi strain.

For the E. coli P_{T5}-ispAdxs P_{T5}-idi strain, the correct integration of the P_{T5} promoter in the front of ispAdxs and idi genes, and elimination of the kanamycin selectable marker from the E. coli chromosome were confirmed by PCR analysis. A colony of the E. coli P_{T5}-ispAdxs P_{T5}-idi strain was resuspended in 50 μL of PCR reaction mixture containing 200 μM dNTPs, 2.5 U AmpliTaq™ (Applied Biosytems), and 0.4 μM of different combination of specific primer pairs, T-kan (5′-ACCGGATATCACCACTTAT CTGCTC-3′; SEQ ID NO:46) and B-ispA (5′-CCTAATAATGCGCCATACTGCATGG-3′; SEQ ID NO:47), T-T5 (5′-TAACCTATAAAAATAGGCGTATCACGAGGCCC-3′; SEQ ID NO:48) and B-ispA, T-kan and B-idi (5′-CAGCCAACTGGAGAACGCGAGATGT-3′; SEQ ID NO:49), T-T5 and B-idi. Test primers were chosen to amplify regions located either in the kanamycin marker or the P_{T5} promoter and the early region of ispAdxs or idi gene (FIG. 3). The PCR reaction was performed as described in Example 1. The PCR results indicated the elimination of the kanamycin selectable marker from the E. coli chromosome (FIG. 3, lane 2 and 4). The chromosomal integration of the P_{T5} promoter fragment upstream of the ispAdxs and idi gene was confirmed based on the expected sizes of PCR products, 285 bp and 274 bp, respectively (FIG. 3, lane 1 and 3).

### Example 7

### Construction of E. coli P_{T5}-ispAdxs P_{T5}-dxs(16a) Strain for Increased β-Carotene Production

In order to construct the E. coli P_{T5}-ispAdxs P_{T5}-dxs(16a) strain containing a chromosomally-integrated P_{T5} promoter upstream from ispAdxs genes and Methylomonas 16a dxs (dxs(16a)), P1 lysate made on E. coli kan-P_{T5}-dxs(16a) strain was transduced into the recipient strain, E. coli kan-P_{T5}-ispAdxs containing a β-carotene biosynthesis expression plasmid pPCB15 (cam^{R}), described in Example 3. Seventy-eight kanamycin-resistance transductants were selected. The kanamycin selectable marker was eliminated from the chromosome of the transductants using a FLP recombinase expression system as described in Example 3, yielding the E. coli P_{T5}-ispAdxs P_{T5}-dxs(16a) strain.

In the E. coli P_{T5}-ispAdxs P_{T5}-dxs(16a) strain the correct integration of the phage T5 promoter in the front of ispAdxs genes and P_{T5}-dxs(16a) at inter-operon region located at 30.9 min on the E. coli chromosome, and elimination of the kanamycin selectable marker were confirmed by PCR analysis. A colony of the E. coli P_{T5}-ispAdxs P_{T5}-dxs(16a) strain was tested by PCR with different combination of specific primer pairs, T-kan and B-ispA, T-T5 and B-ispA, T-kan and B-dxs(16a) (5′-GCGATATTGTATGTCTGATTCAGGA-3′; SEQ ID NO:50), T-T5 and B-dxs(16a). Test primers were chosen to amplify regions located either in the kanamycin resistance gene or the P_{T5} promoter and the downstream region of the chromosomal integration site (FIG. 3). The PCR reaction was performed as described in Example 1. The PCR results indicated the elimination of the kanamycin selectable marker from the E. coli chromosome (FIG. 3, lane 6 and 8). The chromosomal integration of the P_{T5} promoter fragment upstream of the ispAdxs gene and the integration of the P_{T5}-dxs(16a) gene at the inter-operon region was confirmed based on the expected sizes of PCR products, 285 bp and 2184 bp, respectively (FIG. 3, lane 5 and 7).

### Example 8

### Construction of E. coli P_{T5}-ispAdxs P_{T5}-dxs(16a) P_{T5}-lytB(16a) Strain for Increased β-Carotene Production

In order to create a bacterial strain capable of increased carotenoid production, the Methylomonas 16a lytB (lytB(16a)) gene under the control of a P_{T5} promoter was further stacked into the E. coli P_{T5}-ispAdxs P_{T5}-dxs(16a) strain by P1 transduction in combination with the FLP recombination system. P1 lysate made on E. coli kan-P_{T5}-lytB(16a) strain was transduced into the recipient strain, E. coli kan-P_{T5}-ispAdxs kan-P_{T5}-dxs(16a) containing the β-carotene biosynthesis expression plasmid pPCB15 (cam^{R}). Forty-two kanamycin-resistance transductants were selected. The kanamycin selectable marker was eliminated from the chromosome of the transductants using a FLP recombinase expression system as described in Example 6, yielding E. coli P_{T5}-ispAdxs P_{T5}-dxs(16a) P_{T5}-lytB(16a).

For the E. coli P_{T5}-ispAdxs P_{T5}-dxs(16a) P_{T5}-lytB(¹⁶a) strain, the correct integration of the P_{T5} promoter upstream of ispAdxs genes and the addition of the P_{T5}-dxs(16a) and P_{T5}-lytB(16a) genes at inter-operon region located at 30.9 min and 18.1 min, respectively, on the E. coli chromosome, and elimination of the kanamycin selectable marker were confirmed by PCR analysis. A colony of the E. coli P_{T5}-ispAdxs P_{T5}-dxs(16a) P_{T5}-lytB(16a) strain was tested by PCR with different combination of specific primer pairs, T-kan and B-ispA, T-T5 and B-ispA, T-kan and B-dxs(16a), T-T5 and B-dxs(16a), T-kan and B-lytB(16a) (5′-TCCACTGGATGCGGGAAGCTGGCAG-3′; SEQ ID NO:51), T-T5 and B-lytB(16a). Test primers were chosen to amplify regions located either in the kanamycin resistance gene or the P_{T5} promoter and the downstream region of the chromosomal integration site (FIG. 3). The PCR reaction was performed as described in Example 1. The PCR results indicated the elimination of the kanamycin selectable marker from the E. coli chromosome (FIG. 3, lane 10, 12 and 14). The chromosomal integration of the P_{T5} promoter fragment upstream of the ispAdxs gene and integration of the P_{T5}-dxs(16a) and P_{T5}-lytB(16a) genes at the inter-operon region was confirmed based on the expected sizes of PCR products, 285 bp, 2184 bp, and 1282 bp, respectively (FIG. 3, lane 9, 11 and 13).

### Example 9

### Construction of E. coli P_{T5}-ispAdxs P_{T5}-dxs(16a) P_{T5}-lytB(16a) P_{T5}-idi Strain for Increased β-Carotene Production

In order to create a bacterial strain capable of increased carotenoid production, the P_{T5}-idi gene was further stacked into the E. coli P_{T5}-ispAdxs P_{T5}-dxs(16a) P_{T5}-lytB(16a) strain by P1 transduction in combination with the FLP recombination system. P1 lysate made from E. coli kan-P_{T5}-idi strain was transduced into the recipient strain, E. coli kan-P_{T5}-ispAdxs kan-P_{T5}-dxs(16a) P_{T5}-lytB(16a) containing the β-carotene biosynthesis expression plasmid pPCB15. Approximately 450 kanamycin-resistance transductants were selected. The kanamycin selectable marker was eliminated from the chromosome of the transductants using a FLP recombinase expression system as described in Example 6, yielding E. coli P_{T5}-ispAdxs P_{T5}-dxs(16a) P_{T5}-lytB(16a) P_{T5}-idi.

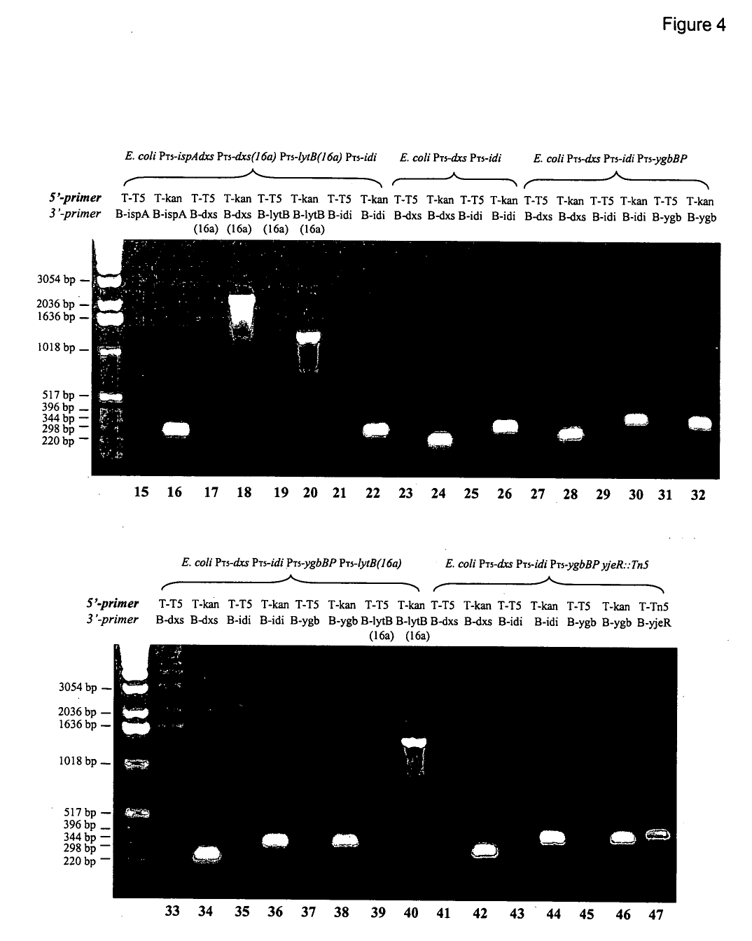

For the E. coli P_{T5}-ispAdxs P_{T5}-dxs(16a) P_{T5}-lytB(16a) P_{T5}-idi strain, the correct integration of the P_{T5} promoter upstream of ispAdxs and idi genes and the integration of the P_{T5}-dxs(16a) and P_{T5}-lytB(16a) genes at inter-operon region located at 30.9 min and 18.1 min, respectively, on the E. coli chromosome, and elimination of the kanamycin selectable marker were confirmed by PCR analysis. A colony of the E. coil P_{T5}-ispAdxs P_{T5}-dxs(16a) P_{T5}-lytB(16a) P_{T5}-idi strain was tested by PCR with different combination of specific primer pairs, T-kan and B-ispA, T-T5 and B-ispA, T-kan and B-dxs(16a), T-T5 and B-dxs(16a), T-kan and B-lytB(16a), T-T5 and B-lytB(16a), T-kan and B-idi, T-T5 and B-idi. Test primers were chosen to amplify regions located either in the kanamycin resistance gene or the P_{T5} promoter and the downstream region of the chromosomal integration site (FIG. 3). The PCR reaction was performed as described in Example 1. The PCR results indicated the elimination of the kanamycin selectable marker from the E. coli chromosome (FIG. 4, lane 16, 18, 20, and 22). The chromosomal integration of the P_{T5} promoter fragment upstream of the ispAdxs and idi genes and the integration of the P_{T5}-dxs(16a) and P_{T5}-lytB(16a) constructs at the inter-operon region was confirmed based on the expected sizes of PCR products, 285 bp, 274 bp, 2184 bp, and 1282 bp, respectively (FIG. 4, lane 15, 17, 19 and 21).

### Example 10

### Construction of E. coli P_{T5}-dxs P_{T5}-idi Strain for Increased β-Carotene Production

In order to characterize the effect of the chromosomal integration of P_{T5} strong promoter in the front of the dxs and idi genes on β-carotene production, E. coli P_{T5}-dxs P_{T5}-idi, capable of producing β-carotene, was constructed.

P1 lysate made with the E. coli kan-P_{T5}-dxs strain was transduced into the recipient strain, E. coli MG1655 containing a β-carotene biosynthesis expression plasmid pPCB15 (cam^{R}) as described in Example 6. Sixteen kanamycin-resistance transductants were selected. The kanamycin selectable marker was eliminated from the chromosome of the transductants using a FLP recombinase expression system, yielding E. coli P_{T5}-dxs strain.

In order to stack kan-P_{T5}-idi on chromosome of E. coli P_{T5}-dxs, P1 lysate made on E. coli kan-P_{T5}-idi strain was transduced into the recipient strain, E. coli P_{T5}-dxs, as described above. Approximately 450 kanamycin-resistance transductants were selected. After transduction, the kanamycin selectable marker was eliminated from the chromosome as described above, yielding E. coli P_{T5}-dxs P_{T5}-idi strain.

For the E. coli P_{T5}-dxs P_{T5}-idi strain, the correct integration of the phage P_{T5} promoter upstream of dxs and idi genes on the E. coli chromosome, and elimination of the kanamycin selectable marker were confirmed by PCR analysis. A colony of the E. coli P_{T5}-dxs P_{T5}-idi strain was tested by PCR with different combination of specific primer pairs, T-kan and B-dxs (5′-TGGCAACAGTCGTAGCTCCTGGGTGG-3′; SEQ ID NO:52), T-T5 and B-dxs, T-kan and B-idi, T-T5 and B-idi. Test primers were chosen to amplify regions located either in the kanamycin or the P_{T5} promoter and the downstream region of the chromosomal integration site (FIG. 3). The PCR reaction was performed as described in Example 1. The PCR results indicated the elimination of the kanamycin selectable marker from the E. coli chromosome (FIG. 4, lane 24 and 26). The chromosomal integration of the P_{T5} promoter fragment upstream of the dxs and idi gene was confirmed based on the expected sizes of PCR products, 229 bp and 274 bp, respectively (FIG. 4, lane 23 and 25).

### Example 11

### Construction of E. coli P_{T5}-dxs P_{T5}-idi P_{T5}-ygbBP Strain for Increased β-Carotene Production

In order to create a bacterial strain capable of increased carotenoid production, P_{T5}-ygbBP gene was further stacked into the E. coli P_{T5}-dxs P_{T5}-idi strain by P1 transduction in combination with the FLP recombination system. P1 lysate was with E. coli kan-P_{T5}-ygbBP strain was transduced into the recipient strain, E. coli kan-P_{T5}-dxs kan-P_{T5}-idi containing the β-carotene biosynthesis expression plasmid pPCB15 (cam^{R}), as described above. Twenty-one kanamycin-resistance transductants were selected. The kanamycin selectable marker was eliminated from the chromosome of the transductants using a FLP recombinase expression system, yielding E. coli P_{T5}-dxs P_{T5}-idi P_{T5}-ygbBP strain.

For the E. coli P_{T5}-dxs P_{T5}-idi P_{T5}-ygbBP strain, the correct integration of the P_{T5} promoter upstream of dxs, idi and ygbBP genes on the E. coli chromosome, and elimination of the kanamycin selectable marker were confirmed by PCR analysis. A colony of the E. coli P_{T5}-dxs P_{T5}-idi P_{T5}-ygbBP strain was tested by PCR with different combination of specific primer pairs, T-kan and B-dxs, T-T5 and B-dxs, T-kan and B-idi, T-T5 and B-idi, T-kan and B-ygb (5′-CCAGCAGCGCATGCACCGAGTGTTC-3′) (SEQ ID NO:53), T-T5 and B-ygb. Test primers were chosen to amplify regions located either in the kanamycin resistance marker or the P_{T5} promoter and the downstream region of the chromosomal integration site (FIG. 3). The PCR reaction was performed as described in Example 1. The PCR results indicated the elimination of the kanamycin selectable marker from the E. coli chromosome (FIG. 4, lane 28, 30 and 32). The chromosomal integration of the P_{T5} promoter fragment upstream of the dxs, idi and ygbBP gene was confirmed based on the expected sizes of PCR products, 229 bp, 274 bp, and 296 bp, respectively (FIG. 4, lane 27, 29, and 31).

### Example 12

### Construction of E. coli P_{T5}-DXS P_{T5}-IDI P_{T5}-ygbBP P_{T5}-lytB(16a) Strain for Increased β-carotene Production

In order to create a bacterial strain capable of increased carotenoid production, the Methylomonas 16a lytB(lytB(16a)) gene under the control of a P_{T5} promoter was further stacked into the E. coli P_{T5}-dxs P_{T5}-idi P_{T5}-ygbBP strain by P1 transduction in combination with the FLP recombination system. P1 lysate made with E. coli kan-P_{T5}-lytB(16a) strain was transduced into the recipient strain, E. coli kan-P_{T5}-dxs kan-P_{T5}-idi P_{T5}-ygbBP containing the β-carotene biosynthesis expression plasmid pPCB15 (cam^{R}), described previously. Approximately 300 kanamycin-resistance transductants were selected. The kanamycin selectable marker was eliminated from the chromosome of the transductants using a FLP recombinase expression system, yielding E. coli P_{T5}-dxs P_{T5}-idi P_{T5}-ygbBP P_{T5}-lytB(¹⁶a) strain.

For the E. coli P_{T5}-dxs P_{T5}-idi P_{T5}-ygbBP P_{T5}-lytB(16a) strain, the correct integration of the P_{T5} promoter upstream of dxs, idi and ygbBP genes and integration of the P_{T5}-lytB(16a) gene at inter-operon region located at 18.1 min on the E. coli chromosome, and elimination of the kanamycin selectable marker were confirmed by PCR analysis. A colony of the E. coli P_{T5}-dxs P_{T5}-idi P_{T5}-ygbBP P_{T5}-lytB(16a) strain was tested by PCR with different combination of specific primer pairs, T-kan and B-dxs, T-T5 and B-dxs, T-kan and B-idi, T-T5 and B-idi, T-kan and B-ygb, T-T5 and B-ygb, T-kan and B-lytB(16a), T-T5 and B-lytB(16a). Test primers were chosen to amplify regions located either in the kanamycin resistance marker or the phage P_{T5} promoter and the downstream region of the chromosomal integration site (FIG. 3). The PCR reaction was performed as described in Example 1. The PCR results indicated the elimination of the kanamycin selectable marker from the E. coli chromosome (FIG. 4, lane 34, 36, 38 and 40). The chromosomal integration of the P_{T5} promoter fragment upstream of the dks, idi and ygbBP gene and the integration of P_{T5}-lytB(16a) gene was confirmed based on the expected sizes of PCR products, 229 bp, 274 bp, 296 bp, and 1282 bp, respectively (FIG. 4, lane 33, 35, 37, and 39).

### Example 13

### Isolation of Chromosomal Mutations that Increase Carotenoid Production

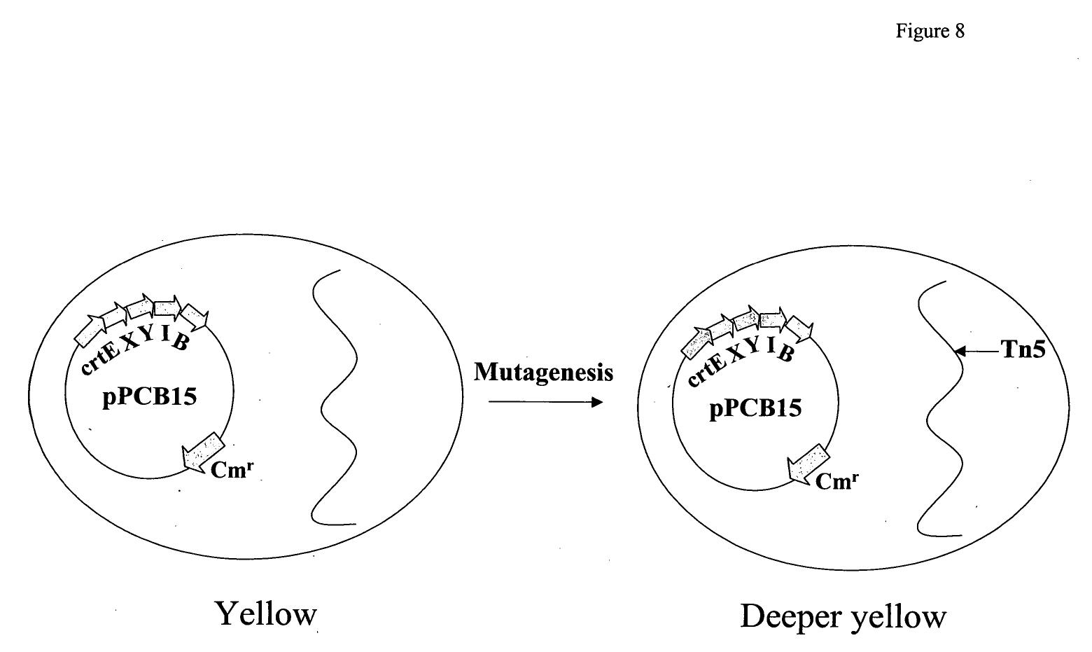

Wild type E. coli is non-carotenogenic and synthesizes only the farnesyl pyrophosphate precursor for carotenoids. When the crtEXYIB gene cluster from Pantoea stewartii was introduced into E. coli, β-carotene was synthesized and the cells exhibit a yellow color characteristic of β-carotene. E. coli chromosomal mutations which increase carotenoid production should result in colonies that have are more intensely pigmented or deeper yellow in color (FIG. 8).

The plasmid pPCB15 (cam^{R}) encodes the carotenoid biosynthesis gene cluster (crtEXYIB) from Pantoea Stewartii (ATCC no. 8199). The pPCB15 plasmid was constructed from ligation of SmaI digested pSU18 (Bartolomeet al., Gene, 102:75-78 (1991)) vector with a blunt-ended PmeI/NotI fragment carrying crtEXYIB from pPCB13 (Example 3). E. coli MG1655 transformed with pPCB15 was used for transposon mutagenesis. Mutagenesis was performed using EZ:TN™ <KAN-2> Tnp Transposome™ kit (Epicentre Technologies, Madison, Wis.) according to manufacture's instructions. The transposon (1 μL) was electroporated into 50 μL of highly electro-competent MG1655 (pPCB15) cells. The mutant cells were spread onto LB-Noble Agar (Difco laboratories, Detroit, Mich.) plates with 25 μg/mL kanamycin and 25 μg/mL chloramphenicol, and grown at 37° C. overnight. Tens of thousands of mutant colonies were visually examined for production of increased levels of β-carotene as evaluated by deeper yellow color development. The candidate mutants were re-streaked to fresh LB-Noble Agar plates and glycerol frozen stocks made for further characterization.

### Example 14

### Quantitation of Carotenoid Production

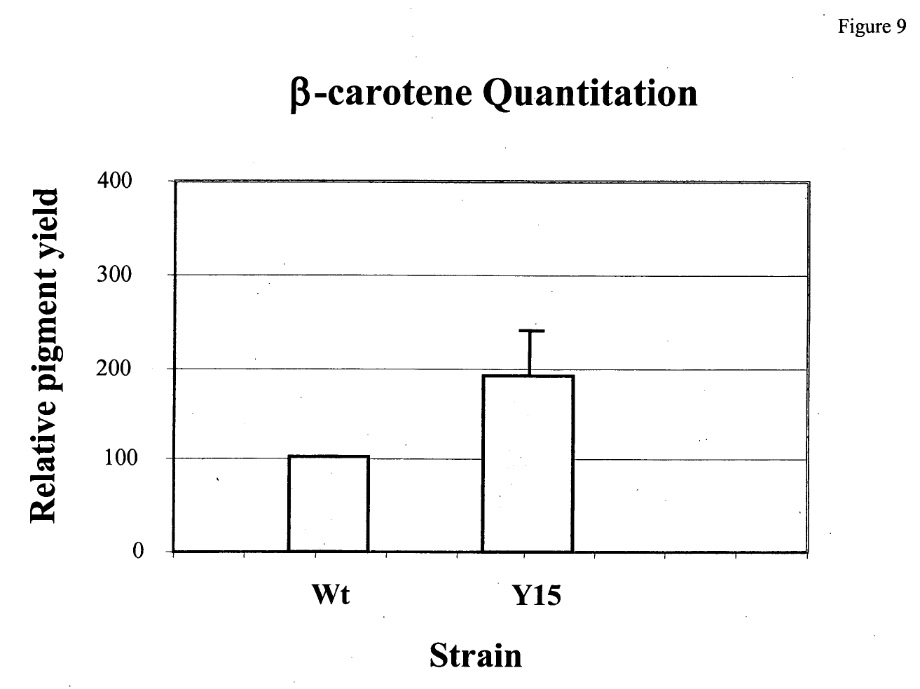

To confirm that the mutants selected for increased production β-carotene by visually screening for deeper yellow colonies in Example 13 indeed produced more β-carotene, the carotenoids were extracted from cultures grown from each mutant strain and quantified spectrophotometrically. Each candidate mutant strain was cultured in 10 mL LB medium with 25 μg/mL chloramphenicol in 50 mL flasks overnight shaking at 250 rpm. MG1655 (pPCB15) was used as the control. Carotenoids were extracted from each cell pellet for 15 min into 1 mL acetone, and the amount of β-carotene produced was measured at 455 nm. Cell density was measured at 600 nm. The ratio OD455/OD600 was used to normalize β-carotene production for different cultures. β-carotene production was also verified by HPLC. Among the mutant clones tested, eight showed increased β-carotene production (FIG. 9). Mutant Y15 showed almost two-fold increase in β-carotene production as shown in FIG. 8 which represents the averages of three independent measurements with standard deviations calculated and indicated as standard deviation bars.

### Example 15

### Mapping of the Transposon Insertions on the E. coli Chromosome

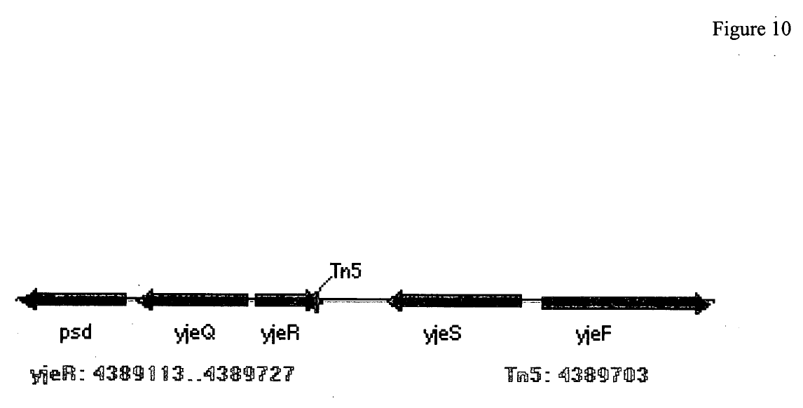

The transposon insertion site in each mutant was identified by PCR and sequencing directly from chromosomal DNA of the mutant strains. A modified single-primer PCR method (Karlyshev et al., BioTechniques, 28:1078-82, 2000) was used. For this method, a 100 μL volume of overnight culture was heated at 99° C. for 10 min in a PCR machine. Cell debris was removed by centrifugation at 4000 g for 10 min. A 1 μL volume of supernatant was used in a 50 μL PCR reaction using either Tn5PCRF (5′-GCTGAGTTGAAGGATCAGATC-3′; SEQ ID NO:54) or Tn5PCRR (5′-CGAGCMGACGTTTCCCGTTG-3′; SEQ ID NO:55) primer. PCR was carried out as follows: 5 min at 95° C.; 20 cycles of 92° C. for 30 sec, 60° C. for 30 sec, 72° C. for 3 min; 30 cycles of 92° C. for 30 sec, 40° C. for 30 sec, 72° C. for 2 min; 30 cycles of 92° C. for 30 sec, 60° C. for 30 sec, 72° C. for 2 min. A 10-μL volume of each PCR product was electrophoresed on an agarose gel to evaluate product length. A 40 μL volume of each PCR product was purified using the Qiagen PCR cleanup kit, and sequenced using sequencing primers Kan-2 FP-1 (5′-ACCTACMCAAAGCTCTCATCMCC-3′; SEQ ID NO:56) or Kan-2 RP-1 (5′-GCMTGTMCATCAGAGATTTTGAG-3′; SEQ ID NO:57) provided by the EZ:TN™ <KAN-2> Tnp Transposome™ kit. The chromosomal insertion site of the transposon was identified as the junction between the Tn5 transposon and MG1655 chromosome DNA by aligning the sequence obtained from each mutant with the E. coli MG1655 genomic sequence. Mutant Y15 carried a Tn5 insertion in yjeR (Ghosh, S., PNAS, 96:4372-4377 (1999)). The Tn5 cassette was located very close to the carboxy terminal end of the gene (FIG. 10) and most likely resulted in functional although truncated protein product.

### Example 16

### Confirmation of Transposon Insertions in E. coli Chromosome

To confirm the transposon insertion sites in Example 15, chromosome specific primers were designed 400-800bp upstream and downstream from the transposon insertion site for each mutant. Primers Y15_F (5′-GGATCGATCTTGAGATGACC-3′; SEQ ID NO:58) and Y15_R (5′-GCTTTCGTAATTTTCGCATTTCTG-3′; SEQ ID NO:59) were used to screen the Y15 mutant. Three sets of PCR reactions were performed for each mutant. The first set (named as PCR 1) uses a chromosome specific upstream primer with a chromosome specific downstream primer. The second set (PCR 2) uses a chromosome specific upstream primer with a transposon specific primer (either Kan-2 FP-1 or Kan-2 RP-1, depending on the orientation of the transposon in the chromosome). The third set (PCR 3) uses a chromosome specific downstream primer with a transposon specific primer. PCR conditions are: 5 min at 95° C.; 30 cycles of 92° C. for 30 sec, 55° C. for 30 sec, 72° C. for 1 min; then 5 min at 72° C. Wild type MG1655 (pPCB15) cells served as control cells. For the control cells, the expected wild type bands were detected in PCR1, and no mutant band was detected in PCR2 or PCR3. For all the eight mutants, no wild type bands were detected in PCR1, and the expected mutant bands were detected in both PCR2 and PCR3. The size of the products in PCR2 and PCR3 correlated well with the insertion sites in each specific gene. Therefore, the mutants contained the transposon insertions as indicated in Example 15.

### Example 17

### Construction of E. coli P_{T5}-dxs P_{T5}-idi P_{T5}-ygbBP yjeR::Tn5 Strain for Increased β-Carotene Production

In order to create a bacterial strain capable of increased carotenoid production, a gene, yjeR::Tn5 (SEQ ID NO:63) partially knocked-out by transposon (Tn5) (kan^{R}) as discovered by experiments outlined in Examples 13-16, was further stacked into the E. coli P_{T5}-dxs P_{T5}-idi P_{T5}-ygbBP strain by P1 transduction. The yjeR gene encoding oligoribonuclease that has a 3′-to-5′ exoribonuclease activity for small oligoribonucleotides has been isolated by random transposon (Tn5)-insertional mutagenesis for increasing β-carotene production. P1 lysate made on E. coli yjeR::Tn5 strain was transduced into the recipient strain, E. coli P_{T5}-dxs P_{T5}-idi P_{T5}-ygbBP containing the β-carotene biosynthesis expression plasmid pPCB15 (cam^{R}), described previously. Six kanamycin-resistance transductants were selected.

For the E. coli P_{T5}-dxs P_{T5}-idi P_{T5}-ygbBP yjeR::Tn5 strain, the correct integration of the P_{T5} promoter upstream of dxs, idi and ygbBP genes and integration of the yjeR::Tn5 gene on the E. coli chromosome was confirmed by PCR fragment analysis. A colony of the E. coli P_{T5}-dxs P_{T5}-idi P_{T5}-ygbBP yjeR::Tn5 strain was tested by PCR with different combination of specific primer pairs, T-kan and B-dxs, T-T5 and B-dxs, T-kan and B-idi, T-T5 and B-idi, T-kan and B-ygb, T-T5 and B-ygb, T-Tn5yjeR (5′-GCAATGTMCATCAGAGATTTTGAG-3′; SEQ ID NO:60) and B-yjeR (5′-GCTTTCGTAATTTTCGCATTTCTG-3′; SEQ ID NO:61). Test primers were chosen to amplify regions located either in the kanamycin selection marker or the P_{T5} promoter and the downstream region of the chromosomal integration site (FIG. 3). The PCR reaction was performed as described in Example 1. The PCR results indicated the elimination of the kanamycin selectable marker from the E. coli chromosome (FIG. 4, lane 42, 44, and 46). The chromosomal integration of the P_{T5} promoter fragment upstream of the dxs, idi and ygbBP genes and the integration of the transposon (Tn5) into yjeR gene (yjeR::Tn5) was confirmed based on the expected sizes of PCR products, 229 bp, 274 bp, 296 bp, and 285 bp, respectively (FIG. 4, lane 41, 43, 45, and 47).

### Example 18

### Construction of E. coli P_{T5}-dxs P_{T5}-idi P_{T5}-ygbBP P_{T5}-ispB Strain for Increased β-Carotene Production

The E. coli P_{T5}-dxs P_{T5}-idi P_{T5}-ygbBP P_{T5}-ispB strain was constructed by P1 transduction in the combination of the Flp site-specific recombinase for marker removal. P1 lysate made from E. coli kan-P_{T5}-ispB strain was transduced into the recipient strain, E. coli P_{T5}-dxs P_{T5}-idi P_{T5}-ygbBP containing the β-carotene biosynthesis expression plasmid pPCB15 (cam^{R}). Thirty-six kanamycin-resistance transductants were selected. A kanamycin selectable marker was eliminated from the chromosome as described at Example 6, yielding E. coli P_{T5}-dxs P_{T5}-idi P_{T5}-ygbBP P_{T5}-ispB.

The stacking of ispB gene under the control of the P_{T5} strong promoter resulted in unexpected increase of β-carotene production. This was a non-obvious result because IspB (octaprenyl diphosphate synthase), which supplies the precursor of the side chain of the isoprenoid quinones, drains away the FPP precursor from the carotenoid biosynthetic pathway (FIG. 1). The mechanism of how overexpression of ispB gene under the control of P_{T5} promoter increases the β-carotene production is not clear yet. However, the result suggests that IspB may increase the flux of the carotenoid biosynthetic pathway.

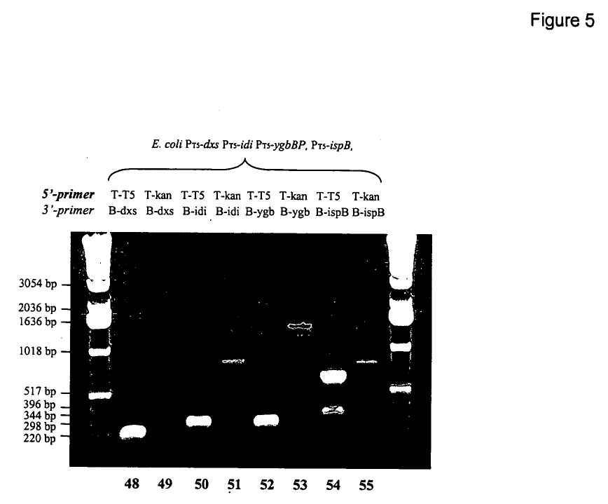

For the E. coli P_{T5}-dxs P_{T5}-idi P_{T5}-ygbBP P_{T5}-ispB strain the correct integration of the phage P_{T5} promoter in the front of dxs, idi, ygbBP, and ispB genes, and elimination of the kanamycin selectable marker were confirmed by PCR analysis. A colony of the E. coli P_{T5}-dxs P_{T5}-idi P_{T5}-ygbBP P_{T5}-ispB was tested by PCR with different combination of specific primer pairs, T-T5 and B-dxs, T-kan and B-dxs, T-T5 and B-idi, T-kan and B-idi, T-T5 and B-ygb, T-kan and B-ygb, T-T5 and B-ispB (5′-AGTACAGCMTCATCGGACGAATACG-3′; SEQ ID NO:62), and T-kan and B-ispB. Test primers were chosen to amplify regions located either in the kanamycin selectable marker or the P_{T5} promoter and the downstream region of the chromosomal integration site (FIG. 3). The PCR reaction was performed as described in Example 1. The PCR results indicated the elimination of the kanamycin selectable marker from the E. coli chromosome (FIG. 5, lane 49, 51, 53, and 55). The chromosomal integration of the P_{T5} promoter upstream of the dxs, idi, ygbBP and ispB genes was confirmed based on the expected sizes of PCR products, 229 bp, 274 bp, 296 bp, and 318 bp, respectively (FIG. 5, lane 48, 50, 52, and 54).

### Example 19

### Transformation of pDCQ108 into E. coli P_{T5}-dxs P_{T5}-idi P_{T5}-ygbBP P_{T5}-IspB Strain

The low copy number plasmid pPCB15 (containing the β-carotene synthesis genes Pantoea crtEXYIB) used as a reporter plasmid for monitoring β-carotene production in E. coli P_{T5}-dxs P_{T5}-idi P_{T5}-ygbBP P_{T5}-ispB was replaced with the medium copy number plasmid PDCQ108 (ATCC PTA-4823) containing β-carotene synthesis genes Pantoea crtEXYIB. The plasmid pPCB15 was eliminated form the E. coli P_{T5}-dxs P_{T5}-idi P_{T5}-ygbBP P_{T5}-ispB strain by streaking on LB plate, incubating at 37° C. for 2 d, and picking up a white-colored colony.

The plasmid pDCQ108 (tet^{R}) was transformed into E. coli P_{T5}-dxs P_{T5}-idi P_{T5}-ygbBP P_{T5}-ispB strain (white colony lacking a carotenoid reporter plasmid). Electro-transformation was performed as described in Example 1. Transformants were selected on 25 μg/mL of tetracycline LB plates at 37° C. The resultant transformants were the E. coli P_{T5}-dxs P_{T5}-idi P_{T5}-ygbBP P_{T5}-ispB strain carrying pDCQ108.

### Example 20

### Measurement of β-Carotene Production in E. coli Strains with Chromosomal Integrations

β-carotene production of the 9 chromosomally engineered E. coli strains, E. coli pPCB15 P_{T5}-ispAdxs P_{T5}-idi, E. coli pPCB15 P_{T5}-ispAdxs P_{T5}-dxs(16a), E. coli pPCB15 P_{T5}-ispAdxs P_{T5}-dxs(16a) P_{T5}-lytB(16a), E. coli μpPCB15 P_{T5}-ispAdxs P_{T5}-dxs(16a) P_{T5}-lytB(16a) P_{T5}-idi, E. coli pPCB15 P_{T5}-dxs P_{T5}-idi, E. coli pPCB15 P_{T5}-dxs P_{T5}-idi P_{T5}-ygbBP, E. coli pPCB15 P_{T5}-dxs P_{T5}-idi P_{T5}-ygbBP P_{T5}-lytB(16a), E. coli pPCB15 P_{T5}-dxs P_{T5}-idi P_{T5}-ygbBP yjeR::Tn5, and E. coli pDCQ108 P_{T5}-dxs P_{T5}-idi P_{T5}-ygbBP P_{T5}-ispB was quantified by the following spectrophotometric method. The quantitative analysis of β-carotene production was achieved by measuring the spectra of β-carotene's characteristic λₘₐₓ peaks at 425, 450 and 478 nm. The 8 chromosomally-engineered E. coli control strains were grown in 5 mL LB containing 25 μg/mL of chloramphenicol at 37° C. for 24 h, and then harvested by centrifugation at 4000 rpm for 10 min. The β-carotene pigment was extracted by resuspending cell pellet in 1 mL of acetone with vortexing for 1 min and then rocking the sample for 1 h at room temperature. Following centrifugation at 4000 rpm for 10 min, the absorption spectrum of the acetone layer containing β-carotene was measured at 450 nm using an Ultrospec 3000 spectrophotometer (Amersham Biosciences, Piscataway, N.J.). The production of β-carotene in E. coli pPCB15 P_{T5}-ispAdxs P_{T5}-idi and E. coli pPCB15 P_{T5}-ispAdxs P_{T5}-dxs(16a) was approximately 3.5-fold and 4.3-fold higher than that of the control strain, E. coli pPCB15, respectively (FIG. 11). Additional stacking of P_{T5}-lytB(16a) and P_{T5}-idi in E. coli pPCB15 P_{T5}-ispAdxs P_{T5}-dxs(16a) P_{T5}-lytB(16a) and E. coli pPCB15 P_{T5}-ispAdxs P_{T5}-dxs(16a) P_{T5}-lytB(16a) P_{T5}-idi didn't increase the production of β-carotene significantly. The production of β-carotene in E. coli pPCB15 P_{T5}-dxs P_{T5}-idi was approximately 4.4-fold higher than that of the E. coli pPCB15 control strain. Additional stacking of P_{T5}-ygbBP and P_{T5}-lytB(16a) in E. coli pPCB15 P_{T5}-dxs P_{T5}-idi P_{T5}-ygbBP and E. coli pPCB15 P_{T5}-dxs P_{T5}-idi P_{T5}-ygbBP increased production of β-carotene 41% and 45%, respectively compared to that of E. coli pPCB15 P_{T5}-dxs P_{T5}-idi (FIG. 11). The production of β-carotene in the E. coli pPCB15 P_{T5}-dxs P_{T5}-idi P_{T5}-ygbBP yjeR::Tn5, was approximately 19-fold higher than that of the E. coli pPCB15 control strain. The E. coli PDCQ108 P_{T5}-dxs P_{T5}-idi P_{T5}-ygbBP P_{T5}-ispB strain showed the best titer of β-carotene production, approximately 30-fold higher than the E. coli pPCB15 control strain.

### Example 21

### Determination of β-Carotene Content in E. coli P_{T5}-dxs P_{T5}-idi P_{T5}-ygbBP yjeR::Tn5 and E. coli P_{T5}-dxs P_{T5}-idi P_{T5}-ygbBP P_{T5}-ispB

Example 20 demonstrated that the E. coli pPCB15 P_{T5}-dxs P_{T5}-idi P_{T5}-ygbBP yjeR::Tn5 (ATCC PTA4807) and E. coli pDCQ108 P_{T5}-dxs P_{T5}-idi P_{T5}-ygbBP P_{T5}-ispB (ATCC PTA-4823) strains in this invention produces high levels of β-carotene, showing deep orange colored colony on LB plate. The content of β-carotene in the E. coli pPCB15 P_{T5}-dxs P_{T5}-idi P_{T5}-ygbBP yjeR::Tn5 and E. coli pDCQ108 P_{T5}-dxs P_{T5}-idi P_{T5}-ygbBP P_{T5}-ispB strains also was quantified by HPLC analysis. The E. coli pPCB15 control, E. coli pCPB15 P_{T5}-dxs P_{T5}-idi P_{T5}-ygbBP yjeR::Tn5 and E. coli pDCQ108 P_{T5}-dxs P_{T5}-idi P_{T5}-ygbBP P_{T5}-ispB strains were grown in 50 mL LB containing 25 μg/mL of chloramphenicol at 37° C. for 24 h with 250 rpm agitation. Twenty mL of the culture cells was filtered on 37 mm diameter cellulose filter (0.2 μm) (Millipore, Bedford, Mass.) that was pre-weighted after drying at 95° C. oven for 24 h. After washing with 10 mL of sterile water, the cells on the pre-weighted filter were completely dried at 95° C. oven for 24 h until its weight did not change. The dry cell weight was determined by subtracting the weight of filter itself from the total weight.

Twenty mL of the culture cells was harvested by centrifugation at 4000 rpm for 10 min for carotenoid extraction and analysis. The β-carotene pigment was extracted as described in Example 20. The carotene extract obtained was analyzed for the β-carotene content by a high performance liquid chromatography (HPLC). A 125×4 mm RP8 (5 μm particles) column (Hewlett-Packard, San Fernando, Calif.) was used for HPLC analysis of β-carotene. The flow rate was 1 mL/min and the solvent program was as follows: 0-11.5 min linear gradient from 40% water/60% methanol to 100% methanol, 11.5-20 min 100% methanol, 20-30 min 40% water/60% methanol. Detection of β-carotene was measured by absorption at 450 nm and quantitative analysis was carried out by comparing an area of the peak of β-carotene to a known β-carotene standard (Sigma, Saint Louis, Mo.).

E. coli pPCB15 P_{T5}-dxs P_{T5}-idi P_{T5}-ygbBP yjeR::Tn5 and E. coli pDCQ108 P_{T5} dxs P_{T5}-idi P_{T5}-ygbBP P_{T5}-ispB strains produced 3.8 mg of β-carotene per gram of dry cell weight (3,800 ppm) and 6.0 mg of β-carotene/g of dry cell weight (6,000 ppm) β-carotene, respectively, while E. coli pPCB15 control strain produces 0.2 mg of β-carotene/g of dry cell weight (200 ppm) (Table 10). The HPLC analysis for the β-carotene content also showed that the chromosomally engineered E. coli pPCB15 P_{T5}-dxs P_{T5}-idi P_{T5}-ygbBP yjeR::Tn5 and E. coli pDCQ108 P_{T5}-dxs P_{T5}-idi P_{T5}-ygbBP P_{T5}-ispB strains produced β-carotene 19-fold and 30-fold higher than the control strain, respectively.

It has been speculated that the limits for carotenoid production in non-carotenogenic host such as E. coli had been reached at the level of around 1.5 mg/g cell dry weight (1,500 ppm) due to overload of the membranes and blocking of membrane functionality (Albrecht et al., supra). The present method has solved the stated problem by making modifications to the E. coli chromosome allowing β-carotene production of 6 mg per g dry weight (6,000 ppm), an increase of 30-fold over initial levels in E. coli pDCQ108 P_{T5}-dxs P_{T5}-idi P_{T5}-ygbBP P_{T5}-ispB.

TABLE 10
β-carotene Production

| Strain | β-Carotene (mg/g dcw¹) |
|---|---|
| E. coli MG1655 pPCB15² | 0.2 |
| E. coli MG1655 pPCB15² | 3.8 |
| P_{T5}-dxs P_{T5}-idi P_{T5}-ygbBP yjeR::Tn5 | |
| E. coli MG1655 pDCQ108³ | 6.0 |
| P_{T5}-dxs P_{T5}-idi P_{T5}-ygbBP P_{T5}-ispB | |

¹Dry Cell Weight
²pPCB15 contains the carotenoid biosynthesis gene cluster (crtEXYIB) from Pantoea Stewartii (ATCC no. 8199).
³pDCQ108 contains the carotenoid biosynthesis gene cluster (crtEXYIB) from Pantoea Stewartii (ATCC no. 8199).

What is claimed is:

1. A carotenoid overproducing bacteria comprising the genes encoding a functional carotenoid enzymatic biosynthetic pathway wherein the dxs, idi and ygbBP genes are overexpressed and wherein the yjeR gene is down regulated.

2. A carotenoid overproducing bacteria comprising the genes encoding a functional carotenoid enzymatic biosynthetic pathway wherein the dxs, idi, ygbBP and ispB genes are overexpressed.

3. The carotenoid overproducing bacteria of claim 1 or 2 wherein the lytB and dxr gene is optionally overexpressed. ispB lytB and dxr yjeR

4. The carotenoid overproducing bacteria of claim 1 or 2 wherein the carotenoid enzymatic biosynthetic pathway consists of the genes dxs, dxr, ygpP, ychB, ygbB, lytB, idi, ispA, ispB crtE, crtB, crtI, and crtY.

5. The carotenoid overproducing bacteria of claim 4 wherein the carotenoid enzymatic biosynthetic pathway optionally additionally comprises the crtZ and crtW genes.

6. The carotenoid overproducing bacteria of any of claims 1-5 wherein the bacteria is selected from the group consisting Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Paracoccus, Escherichia, Bacillus, Myxococcus, Salmonella, Yersinia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus.

7. The carotenoid overproducing bacteria of claim 6 wherein the bactera is E. coli.

8. The carotenoid overproducing bacteria of claims 1-3 wherein the dxs, dxr, ygpP, ychB, ygbB, lytB, idi, ispA, ispB are derived from a Methylomonas sp.

9. The carotenoid overproducing bacteria of any of claims 1-3 wherein the dxs, idi, ispB and ygbBP genes are under the control of a strong promoter.

10. The carotenoid overproducing bacteria of claim 9 wherein the strong promoter is selected from the group consisting of lac, ara, tet, trp, λP_{L}, λP_{R}, T7, tac, P_{T5}, and trc.

11. The carotenoid overproducing bacteria of any of claims 1-3 wherein the dxs, idi, ispB and ygbBP genes are integrated in multicopy in the bacterial chromosome.

12. The carotenoid overproducing bacteria of any of claims 1-3 wherein the dxs, idi, ispB and ygbBP genes are present in multicopy in the bacteria on one or more plasmids.

13. The carotenoid overproducing bacteria of of claim 7 wherein the yjeR gene is down regulated by gene disruption.

14. The carotenoid overproducing bacteria of claim 13 wherein the disrupted yjeR gene has the nucleotide sequence as set forth in SEQ ID NO:63.

15. The carotenoid overproducing bacteria of either of any of claims 1-3 wherein the dxs, idi, ispB ygbBP and lytB genes are chromosomally integrated into the host cell genome.

16. A carotenoid overproducing bacteria selected from the group consisting of: a strain having the ATCC identification number PTA-4807 and a strain having the ATCC identification number PTA-4823.

17. A method for the production of a carotenoid comprising:
a) growing the carotenoid overproducing bacteria of any of claims 1-5, the bacteria overexpressing at least one gene selected from the group consisting of dxs, idi ygbBP, ispB, lytB, dxr, wherein yjeR is optionally downregulated, for a time sufficient to produce a carotenoid; and
b) optionally recovering the carotenoid from the carotenoid overproducing bacteria of step (a).

18. A method according to claim 17 wherein the carotenoid is selected from the group consisting of antheraxanthin, adonixanthin, astaxanthin, canthaxanthin, capsorubrin, β-cryptoxanthin, didehydrolycopene, didehydrolycopene, β-carotene, ζ-carotene, δ-carotene, γ-carotene, keto-γ-carotene, ψ-carotene, ε-carotene, β,ψ-carotene, torulene, echinenone, gamma-carotene, zeta-carotene, alpha-cryptoxanthin, diatoxanthin, 7,8-didehydroastaxanthin, fucoxanthin, fucoxanthinol, isorenieratene, β-isorenieratene lactucaxanthin, lutein, lycopene, neoxanthin, neurosporene, hydroxyneurosporene, peridinin, phytoene, rhodopin, rhodopin glucoside, siphonaxanthin, spheroidene, spheroidenone, spirilloxanthin, uriolide, uriolide acetate, violaxanthin, zeaxanthin-β-diglucoside, zeaxanthin, and C30-carotenoids.

19. A method according to claim 18 wherein the carotenoid is produced at a level of at least about 6 mg per gram dry cell weight.

20. A method according to claim 18 wherein the bacteria is selected from the group consisting Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Paracoccus, Escherichia, Bacillus, Myxococcus, Salmonella, Yersinia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus.

21. A method according to claim 20 wherein the bacteria is E. coli.

22. A method according to claim 17 wherein the dxs, idi, ygbBP, ispB and lytB genes are under the control of a promoter selected from the group consisting of lac, ara, tet, trp, λP_{L}, λP_{R}, T7, tac, P_{T5}, and trc.

23. A method according to claim 17 wherein the dxs, idi, ispB, ygbBP and lytB genes are integrated in multicopy in the bacterial chromosome.

24. A method according to claim 17 wherein the dxs, idi, ispB, ygbBP and lytB genes are in multicopy in the bacteria on one or more plasmids.

25. A method according to claim 17 wherein the yjeR gene is down regulated by gene disruption.

26. A method according to claim 25 wherein the disrupted yjeR gene has the nucleotide sequence as set forth in SEQ ID NO:63.

27. A method according to claim 17 wherein the dxs, idi ispB, ygbBP and lytB genes are chromosomally integrated into the host cell genome.


SEQUENCE LISTING (66 sequences)

SEQ ID NO: 1 - DNA, 912 bp, Pantoea stewartii
  misc_feature (1)..(3): ttg alternative start codon used to encode methionine
ttgacggtctgcgcaaaaaaacacgttcaccttactggcatttcggctgagcagttgctggctgatatcgatagccgccttgatcagttactgccggttcagggtgagcgggattgtgtgggtgccgcgatgcgtgaaggcacgctggcaccgggcaaacgtattcgtccgatgctgctgttattaacagcgcgcgatcttggctgtgcgatcagtcacgggggattactggatttagcctgcgcggttgaaatggtgcatgctgcctcgctgattctggatgatatgccctgcatggacgatgcgcagatgcgtcgggggcgtcccaccattcacacgcagtacggtgaacatgtggcgattctggcggcggtcgctttactcagcaaagcgtttggggtgattgccgaggctgaaggtctgacgccgatagccaaaactcgcgcggtgtcggagctgtccactgcgattggcatgcagggtctggttcagggccagtttaaggacctctcggaaggcgataaaccccgcagcgccgatgccatactgctaaccaatcagtttaaaaccagcacgctgttttgcgcgtcaacgcaaatggcgtccattgcggccaacgcgtcctgcgaagcgcgtgagaacctgcatcgtttctcgctcgatctcggccaggcctttcagttgcttgacgatcttaccgatggcatgaccgataccggcaaagacatcaatcaggatgcaggtaaatcaacgctggtcaatttattaggctcaggcgcggtcgaagaacgcctgcgacagcatttgcgcctggccagtgaacacctttccgcggcatgccaaaacggccattccaccacccaactttttattcaggcctggtttgacaaaaaactcgctgccgtcagttaa

SEQ ID NO: 2 - protein, 303 aa, Pantoea stewartii
MTVCAKKHVHLTGISAEQLLADIDSRLDQLLPVQGERDCVGAAMREGTLAPGKRIRPMLLLLTARDLGCAISHGGLLDLACAVEMVHAASLILDDMPCMDDAQMRRGRPTIHTQYGEHVAILAAVALLSKAFGVIAEAEGLTPIAKTRAVSELSTAIGMQGLVQGQFKDLSEGDKPRSADAILLTNQFKTSTLFCASTQMASIAANASCEARENLHRFSLDLGQAFQLLDDLTDGMTDTGKDINQDAGKSTLVNLLGSGAVEERLRQHLRLASEHLSAACQNGHSTTQLFIQAWFDKKLAAVS

SEQ ID NO: 3 - DNA, 1296 bp, Pantoea stewartii
  CDS (1)..(1296)
atgagccattttgcggtgatcgcaccgccctttttcagccatgttcgcgctctgcaaaaccttgctcaggaattagtggcccgcggtcatcgtgttacgttttttcagcaacatgactgcaaagcgctggtaacgggcagcgatatcggattccagaccgtcggactgcaaacgcatcctcccggttccttatcgcacctgctgcacctggccgcgcacccactcggaccctcgatgttacgactgatcaatgaaatggcacgtaccagcgatatgctttgccgggaactgcccgccgcttttcatgcgttgcagatagagggcgtgatcgttgatcaaatggagccggcaggtgcagtagtcgcagaagcgtcaggtctgccgtttgtttcggtggcctgcgcgctgccgctcaaccgcgaaccgggtttgcctctggcggtgatgcctttcgagtacggcaccagcgatgcggctcgggaacgctataccaccagcgaaaaaatttatgactggctgatgcgacgtcacgatcgtgtgatcgcgcatcatgcatgcagaatgggtttagccccgcgtgaaaaactgcatcattgtttttctccactggcacaaatcagccagttgatccccgaactggattttccccgcaaagcgctgccagactgctttcatgcggttggaccgttacggcaaccccaggggacgccggggtcatcaacttcttattttccgtccccggacaaaccccgtatttttgcctcgctgggcaccctgcagggacatcgttatggcctgttcaggaccatcgccaaagcctgcgaagaggtggatgcgcagttactgttggcacactgtggcggcctctcagccacgcaggcaggtgaactggcccggggcggggacattcaggttgtggattttgccgatcaatccgcagcactttcacaggcacagttgacaatcacacatggtgggatgaatacggtactggacgctattgcttcccgcacaccgctactggcgctgccgctggcatttgatcaacctggcgtggcatcacgaattgtttatcatggcatcggcaagcgtgcgtctcggtttactaccagccatgcgctggcgcggcagattcgatcgctgctgactaacaccgattacccgcagcgtatgacaaaaattcaggccgcattgcgtctggcaggcggcacaccagccgccgccgatattgttgaacaggcgatgcggacctgtcagccagtactcagtgggcaggattatgcaaccgcactatga

SEQ ID NO: 4 - protein, 431 aa, Pantoea stewartii
MSHFAVIAPPFFSHVRALQNLAQELVARGHRVTFFQQHDCKALVTGSDIGFQTVGLQTHPPGSLSHLLHLAAHPLGPSMLRLINEMARTSDMLCRELPAAFHALQIEGVIVDQMEPAGAVVAEASGLPFVSVACALPLNREPGLPLAVMPFEYGTSDAARERYTTSEKIYDWLMRRHDRVIAHHACRMGLAPREKLHHCFSPLAQISQLIPELDFPRKALPDCFHAVGPLRQPQGTPGSSTSYFPSPDKPRIFASLGTLQGHRYGLFRTIAKACEEVDAQLLLAHCGGLSATQAGELARGGDIQVVDFADQSAALSQAQLTITHGGMNTVLDAIASRTPLLALPLAFDQPGVASRIVYHGIGKRASRFTTSHALARQIRSLLTNTDYPQRMTKIQAALRLAGGTPAAADIVEQAMRTCQPVLSGQDYATAL

SEQ ID NO: 5 - DNA, 1149 bp, Pantoea stewartii
  CDS (1)..(1149)
atgcaaccgcactatgatctcattctggtcggtgccggtctggctaatggccttatcgcgctccggcttcagcaacagcatccggatatgcggatcttgcttattgaggcgggtcctgaggcgggagggaaccatacctggtcctttcacgaagaggatttaacgctgaatcagcatcgctggatagcgccgcttgtggtccatcactggcccgactaccaggttcgtttcccccaacgccgtcgccatgtgaacagtggctactactgcgtgacctcccggcatttcgccgggatactccggcaacagtttggacaacatttatggctgcataccgcggtttcagccgttcatgctgaatcggtccagttagcggatggccggattattcatgccagtacagtgatcgacggacggggttacacgcctgattctgcactacgcgtaggattccaggcatttatcggtcaggagtggcaactgagcgcgccgcatggtttatcgtcaccgattatcatggatgcgacggtcgatcagcaaaatggctaccgctttgtttataccctgccgctttccgcaaccgcactgctgatcgaagacacacactacattgacaaggctaatcttcaggccgaacgggcgcgtcagaacattcgcgattatgctgcgcgacagggttggccgttacagacgttgctgcgggaagaacagggtgcattgcccattacgttaacgggcgataatcgtcagttttggcaacagcaaccgcaagcctgtagcggattacgcgccgggctgtttcatccgacaaccggctactccctaccgctcgcggtggcgctggccgatcgtctcagcgcgctggatgtgtttacctcttcctctgttcaccagacgattgctcactttgcccagcaacgttggcagcaacaggggtttttccgcatgctgaatcgcatgttgtttttagccggaccggccgagtcacgctggcgtgtgatgcagcgtttctatggcttacccgaggatttgattgcccgcttttatgcgggaaaactcaccgtgaccgatcggctacgcattctgagcggcaagccgcccgttcccgttttcgcggcattgcaggcaattatgacgactcatcgttga

SEQ ID NO: 6 - protein, 382 aa, Pantoea stewartii
MQPHYDLILVGAGLANGLIALRLQQQHPDMRILLIEAGPEAGGNHTWSFHEEDLTLNQHRWIAPLVVHHWPDYQVRFPQRRRHVNSGYYCVTSRHFAGILRQQFGQHLWLHTAVSAVHAESVQLADGRIIHASTVIDGRGYTPDSALRVGFQAFIGQEWQLSAPHGLSSPIIMDATVDQQNGYRFVYTLPLSATALLIEDTHYIDKANLQAERARQNIRDYAARQGWPLQTLLREEQGALPITLTGDNRQFWQQQPQACSGLRAGLFHPTTGYSLPLAVALADRLSALDVFTSSSVHQTIAHFAQQRWQQQGFFRMLNRMLFLAGPAESRWRVMQRFYGLPEDLIARFYAGKLTVTDRLRILSGKPPVPVFAALQAIMTTHR

SEQ ID NO: 7 - DNA, 1479 bp, Pantoea stewartii
  CDS (1)..(1479)
atgaaaccaactacggtaattggtgcgggctttggtggcctggcactggcaattcgtttacaggccgcaggtattcctgttttgctgcttgagcagcgcgacaagccgggtggccgggcttatgtttatcaggagcagggctttacttttgatgcaggccctaccgttatcaccgatcccagcgcgattgaagaactgtttgctctggccggtaaacagcttaaggattacgtcgagctgttgccggtcacgccgttttatcgcctgtgctgggagtccggcaaggtcttcaattacgataacgaccaggcccagttagaagcgcagatacagcagtttaatccgcgcgatgttgcgggttatcgagcgttccttgactattcgcgtgccgtattcaatgagggctatctgaagctcggcactgtgccttttttatcgttcaaagacatgcttcgggccgcgccccagttggcaaagctgcaggcatggcgcagcgtttacagtaaagttgccggctacattgaggatgagcatcttcggcaggcgttttcttttcactcgctcttagtgggggggaatccgtttgcaacctcgtccatttatacgctgattcacgcgttagaacgggaatggggcgtctggtttccacgcggtggaaccggtgcgctggtcaatggcatgatcaagctgtttcaggatctgggcggcgaagtcgtgcttaacgcccgggtcagtcatatggaaaccgttggggacaagattcaggccgtgcagttggaagacggcagacggtttgaaacctgcgcggtggcgtcgaacgctgatgttgtacatacctatcgcgatctgctgtctcagcatcccgcagccgctaagcaggcgaaaaaactgcaatccaagcgtatgagtaactcactgtttgtactctattttggtctcaaccatcatcacgatcaactcgcccatcataccgtctgttttgggccacgctaccgtgaactgattcacgaaatttttaaccatgatggtctggctgaggatttttcgctttatttacacgcaccttgtgtcacggatccgtcactggcaccggaagggtgcggcagctattatgtgctggcgcctgttccacacttaggcacggcgaacctcgactgggcggtagaaggaccccgactgcgcgatcgtatttttgactaccttgagcaacattacatgcctggcttgcgaagccagttggtgacgcaccgtatgtttacgccgttcgatttccgcgacgagctcaatgcctggcaaggttcggccttctcggttgaacctattctgacccagagcgcctggttccgaccacataaccgcgataagcacattgataatctttatctggttggcgcaggcacccatcctggcgcgggcattcccggcgtaatcggctcggcgaaggcgacggcaggcttaatgctggaggacctgatttga

SEQ ID NO: 8 - protein, 492 aa, Pantoea stewartii
MKPTTVIGAGFGGLALAIRLQAAGIPVLLLEQRDKPGGRAYVYQEQGFTFDAGPTVITDPSAIEELFALAGKQLKDYVELLPVTPFYRLCWESGKVFNYDNDQAQLEAQIQQFNPRDVAGYRAFLDYSRAVFNEGYLKLGTVPFLSFKDMLRAAPQLAKLQAWRSVYSKVAGYIEDEHLRQAFSFHSLLVGGNPFATSSIYTLIHALEREWGVWFPRGGTGALVNGMIKLFQDLGGEVVLNARVSHMETVGDKIQAVQLEDGRRFETCAVASNADVVHTYRDLLSQHPAAAKQAKKLQSKRMSNSLFVLYFGLNHHHDQLAHHTVCFGPRYRELIHEIFNHDGLAEDFSLYLHAPCVTDPSLAPEGCGSYYVLAPVPHLGTANLDWAVEGPRLRDRIFDYLEQHYMPGLRSQLVTHRMFTPFDFRDELNAWQGSAFSVEPILTQSAWFRPHNRDKHIDNLYLVGAGTHPGAGIPGVIGSAKATAGLMLEDLI

SEQ ID NO: 9 - DNA, 891 bp, Pantoea stewartii
  CDS (1)..(891)
atggcggttggctcgaaaagctttgcgactgcatcgacgcttttcgacgccaaaacccgtcgcagcgtgctgatgctttacgcatggtgccgccactgcgacgacgtcattgacgatcaaacactgggctttcatgccgaccagccctcttcgcagatgcctgagcagcgcctgcagcagcttgaaatgaaaacgcgtcaggcctacgccggttcgcaaatgcacgagcccgcttttgccgcgtttcaggaggtcgcgatggcgcatgatatcgctcccgcctacgcgttcgaccatctggaaggttttgccatggatgtgcgcgaaacgcgctacctgacactggacgatacgctgcgttattgctatcacgtcgccggtgttgtgggcctgatgatggcgcaaattatgggcgttcgcgataacgccacgctcgatcgcgcctgcgatctcgggctggctttccagttgaccaacattgcgcgtgatattgtcgacgatgctcaggtgggccgctgttatctgcctgaaagctggctggaagaggaaggactgacgaaagcgaattatgctgcgccagaaaaccggcaggccttaagccgtatcgccgggcgactggtacgggaagcggaaccctattacgtatcatcaatggccggtctggcacaattacccttacgctcggcctgggccatcgcgacagcgaagcaggtgtaccgtaaaattggcgtgaaagttgaacaggccggtaagcaggcctgggatcatcgccagtccacgtccaccgccgaaaaattaacgcttttgctgacggcatccggtcaggcagttacttcccggatgaagacgtatccaccccgtcctgctcatctctggcagcgcccgatctag

SEQ ID NO: 10 - protein, 296 aa, Pantoea stewartii
MAVGSKSFATASTLFDAKTRRSVLMLYAWCRHCDDVIDDQTLGFHADQPSSQMPEQRLQQLEMKTRQAYAGSQMHEPAFAAFQEVAMAHDIAPAYAFDHLEGFAMDVRETRYLTLDDTLRYCYHVAGVVGLMMAQIMGVRDNATLDRACDLGLAFQLTNIARDIVDDAQVGRCYLPESWLEEEGLTKANYAAPENRQALSRIAGRLVREAEPYYVSSMAGLAQLPLRSAWAIATAKQVYRKIGVKVEQAGKQAWDHRQSTSTAEKLTLLLTASGQAVTSRMKTYPPRPAHLWQRPI

SEQ ID NO: 11 - DNA, 528 bp, Pantoea stewartii
  CDS (1)..(528)
atgttgtggatttggaatgccctgatcgtgtttgtcaccgtggtcggcatggaagtggttgctgcactggcacataaatacatcatgcacggctggggttggggctggcatctttcacatcatgaaccgcgtaaaggcgcatttgaagttaacgatctctatgccgtggtattcgccattgtgtcgattgccctgatttacttcggcagtacaggaatctggccgctccagtggattggtgcaggcatgaccgcttatggtttactgtattttatggtccacgacggactggtacaccagcgctggccgttccgctacataccgcgcaaaggctacctgaaacggttatacatggcccaccgtatgcatcatgctgtaaggggaaaagagggctgcgtgtcctttggttttctgtacgcgccaccgttatctaaacttcaggcgacgctgagagaaaggcatgcggctagatcgggcgctgccagagatgagcaggacggggtggatacgtcttcatccgggaagtaa

SEQ ID NO: 12 - protein, 175 aa, Pantoea stewartii
MLWIWNALIVFVTVVGMEVVAALAHKYIMHGWGWGWHLSHHEPRKGAFEVNDLYAVVFAIVSIALIYFGSTGIWPLQWIGAGMTAYGLLYFMVHDGLVHQRWPFRYIPRKGYLKRLYMAHRMHHAVRGKEGCVSFGFLYAPPLSKLQATLRERHAARSGAARDEQDGVDTSSSGK

SEQ ID NO: 13 - DNA, 1860 bp, Methylomonas 16a
  CDS (1)..(1860)
atggctctttccaaagacttccctctactcaattccatccacaccccagcggacatacgcgcgctgtccaaggaccagctccagcaactggctgacgaggtgcgcggctatctgacccacacggtcagcatttccggcggccattttgcggccggcctcggcaccgtggaactgaccgtggccttgcattatgtgttcaatacccccgtcgatcagttggtctgggacgtgggccatcaggcctatccgcacaagattctgaccggtcgcaaggagcgcatgccgaccattcgcaccctgggcggggtgtcagcctttccggcgcgggacgagagcgaatacgatgccttcggcgtcggccattccagcacctcgatcagcgcggcactgggcatggccattgcgtcgcagctgcgcggcgaagacaagaagatggtagccatcatcggcgacggttccatcaccggcggcatggcctatgaggcgatgaatcatgccggcgatgtgaatgccaacctgctggtgatcttgaacgacaacgatatgtcgatctcgccgccggtcggggcgatgaacaattatctgaccaaggtgttgtcgagcaagttttattcgtcggtgcgggaagagagcaagaaagctctggccaagatgccgtcggtgtgggaactggcgcgcaagaccgaggaacacgtgaagggcatgatcgtgcccggtaccttgttcgaggaattgggcttcaattatttcggcccgatcgacggccatgatgtcgagatgctggtgtcgaccctggaaaatctgaaggatttgaccgggccggtattcctgcatgtggtgaccaagaagggcaaaggctatgcgccagccgagaaagacccgttggcctaccatggcgtgccggctttcgatccgaccaaggatttcctgcccaaggcggcgccgtcgccgcatccgacctataccgaggtgttcggccgctggctgtgcgacatggcggctcaagacgagcgcttgctgggcatcacgccggcgatgcgcgaaggctctggtttggtggaattctcacagaaatttccgaatcgctatttcgatgtcgccatcgccgagcagcatgcggtgaccttggccgccggccaggcctgccagggcgccaagccggtggtggcgatttattccaccttcctgcaacgcggttacgatcagttgatccacgacgtggccttgcagaacttagatatgctctttgcactggatcgtgccggcttggtcggcccggatggaccgacccatgctggcgcctttgattacagctacatgcgctgtattccgaacatgctgatcatggctccagccgacgagaacgagtgcaggcagatgctgaccaccggcttccaacaccatggcccggcttcggtgcgctatccgcgcggcaaagggcccggggcggcaatcgatccgaccctgaccgcgctggagatcggcaaggccgaagtcagacaccacggcagccgcatcgccattctggcctggggcagcatggtcacgcctgccgtcgaagccggcaagcagctgggcgcgacggtggtgaacatgcgtttcgtcaagccgttcgatcaagccttggtgctggaattggccaggacgcacgatgtgttcgtcaccgtcgaggaaaacgtcatcgccggcggcgctggcagtgcgatcaacaccttcctgcaggcgcagaaggtgctgatgccggtctgcaacatcggcctgcccgaccgcttcgtcgagcaaggtagtcgcgaggaattgctcagcctggtcggcctcgacagcaagggcatcttcgccaccatcgaacagttttgcgct

SEQ ID NO: 14 - protein, 620 aa, Methylomonas 16a
MALSKDFPLLNSIHTPADIRALSKDQLQQLADEVRGYLTHTVSISGGHFAAGLGTVELTVALHYVFNTPVDQLVWDVGHQAYPHKILTGRKERMPTIRTLGGVSAFPARDESEYDAFGVGHSSTSISAALGMAIASQLRGEDKKMVAIIGDGSITGGMAYEAMNHAGDVNANLLVILNDNDMSISPPVGAMNNYLTKVLSSKFYSSVREESKKALAKMPSVWELARKTEEHVKGMIVPGTLFEELGFNYFGPIDGHDVEMLVSTLENLKDLTGPVFLHVVTKKGKGYAPAEKDPLAYHGVPAFDPTKDFLPKAAPSPHPTYTEVFGRWLCDMAAQDERLLGITPAMREGSGLVEFSQKFPNRYFDVAIAEQHAVTLAAGQACQGAKPVVAIYSTFLQRGYDQLIHDVALQNLDMLFALDRAGLVGPDGPTHAGAFDYSYMRCIPNMLIMAPADENECRQMLTTGFQHHGPASVRYPRGKGPGAAIDPTLTALEIGKAEVRHHGSRIAILAWGSMVTPAVEAGKQLGATVVNMRFVKPFDQALVLELARTHDVFVTVEENVIAGGAGSAINTFLQAQKVLMPVCNIGLPDRFVEQGSREELLSLVGLDSKGIFATIEQFCA

SEQ ID NO: 15 - DNA, 982 bp, Methylomonas 16a
  CDS (22)..(975)
cccagtaaaacactcaagaatatgcaaatcgtactcgcaaacccccgtggattctgtgccggcgtggaccgggccattgaaattgtcgatcaagccatcgaagcctttggtgcgccgatttatgtgcggcacgaggtggtgcataaccgcaccgtggtcgatggactgaaacaaaaaggtgcggtgttcatcgaggaactaagcgatgtgccggtgggttcctacttgattttcagcgcgcacggcgtatccaaggaggtgcaacaggaagccgaggagcgccagttgacggtattcgatgcgacttgtccgctggtgaccaaagtgcacatgcaggttgccaagcatgccaaacagggccgagaagtgattttgatcggccacgccggtcatccggaagtggaaggcacgatgggccagtatgaaaaatgcaccgaaggcggcggcatttatctggtcgaaactccggaagacgtacgcaatttgaaagtcaacaatcccaatgatctggcctatgtgacgcagacgaccttgtcgatgaccgacaccaaggtcatggtggatgcgttacgcgaacaatttccgtccattaaggagcaaaaaaaggacgatatttgttacgcgacgcaaaaccgtcaggatgcggtgcatgatctggccaagatttccgacctgattctggttgtcggctctcccaatagttcgaattccaaccgtttgcgtgaaatcgccgtgcaactcggtaaacccgcttatttgatcgatacttaccaggatttgaagcaagattggctggagggaattgaagtagtcggggttaccgcgggcgcttcggcgccggaagtgttggtgcaggaagtgatcgatcaactgaaggcatggggcggcgaaaccacttcggtcagagaaaacagcggcatcgaggaaaaggtagtcttttcgattcccaaggagttgaaaaaacatatgcaagcgtgatcaa

SEQ ID NO: 16 - protein, 318 aa, Methylomonas 16a
MQIVLANPRGFCAGVDRAIEIVDQAIEAFGAPIYVRHEVVHNRTVVDGLKQKGAVFIEELSDVPVGSYLIFSAHGVSKEVQQEAEERQLTVFDATCPLVTKVHMQVAKHAKQGREVILIGHAGHPEVEGTMGQYEKCTEGGGIYLVETPEDVRNLKVNNPNDLAYVTQTTLSMTDTKVMVDALREQFPSIKEQKKDDICYATQNRQDAVHDLAKISDLILVVGSPNSSNSNRLREIAVQLGKPAYLIDTYQDLKQDWLEGIEVVGVTAGASAPEVLVQEVIDQLKAWGGETTSVRENSGIEEKVVFSIPKELKKHMQA

SEQ ID NO: 17 - DNA, 1254 bp, Methylomonas 16a
  CDS (73)..(1254)
ggtggacagcatcattgcggcggcaccgtttttctatgccggtatcgtgctgatcggacggagcgtattcgaatgaaaggtatttgcatattgggcgctaccggttcgatcggtgtcagcacgctggatgtcgttgccaggcatccggataaatatcaagtcgttgcgctgaccgccaacggcaatatcgacgcattgtatgaacaatgcctggcccaccatccggagtatgcggtggtggtcatggaaagcaaggtagcagagttcaaacagcgcattgccgcttcgccggtagcggatatcaaggtcttgtcgggtagcgaggccttgcaacaggtggccacgctggaaaacgtcgatacggtgatggcggctatcgtcggcgcggccggattgttgccgaccttggccgcggccaaggccggcaaaaccgtgctgttggccaacaaggaagccttggtgatgtcgggacaaatcttcatgcaggccgtcagcgattccggcgctgtgttgctgccgatagacagcgagcacaacgccatctttcagtgcatgccggcgggttatacgccaggccatacagccaaacaggcgcgccgcattttattgaccgcttccggtggcccatttcgacggacgccgatagaaacgttgtccagcgtcacgccggatcaggccgttgcccatcctaaatgggacatggggcgcaagatttcggtcgattccgccaccatgatgaacaaaggtctcgaactgatcgaagcctgcttgttgttcaacatggagcccgaccagattgaagtcgtcattcatccgcagagcatcattcattcgatggtggactatgtcgatggttcggttttggcgcagatgggtaatcccgacatgcgcacgccgatagcgcacgcgatggcctggccggaacgctttgactctggtgtggcgccgctggatattttcgaagtagggcacatggatttcgaaaaacccgacttgaaacggtttccttgtctgagattggcttatgaagccatcaagtctggtggaattatgccaacggtattgaacgcagccaatgaaattgctgtcgaagcgtttttaaatgaagaagtcaaattcactgacatcgcggtcatcatcgagcgcagcatggcccagtttaaaccggacgatgccggcagcctcgaattggttttgcaggccgatcaagatgcgcgcgaggtggctagagacatcatcaagaccttggtagct

SEQ ID NO: 18 - protein, 394 aa, Methylomonas 16a
MKGICILGATGSIGVSTLDVVARHPDKYQVVALTANGNIDALYEQCLAHHPEYAVVVMESKVAEFKQRIAASPVADIKVLSGSEALQQVATLENVDTVMAAIVGAAGLLPTLAAAKAGKTVLLANKEALVMSGQIFMQAVSDSGAVLLPIDSEHNAIFQCMPAGYTPGHTAKQARRILLTASGGPFRRTPIETLSSVTPDQAVAHPKWDMGRKISVDSATMMNKGLELIEACLLFNMEPDQIEVVIHPQSIIHSMVDYVDGSVLAQMGNPDMRTPIAHAMAWPERFDSGVAPLDIFEVGHMDFEKPDLKRFPCLRLAYEAIKSGGIMPTVLNAANEIAVEAFLNEEVKFTDIAVIIERSMAQFKPDDAGSLELVLQADQDAREVARDIIKTLVA

SEQ ID NO: 19 - DNA, 25 bp, Artificial sequence
  Primer #1 for amplification of crt gene cluster
atgacggtctgcgcaaaaaaacacg

SEQ ID NO: 20 - DNA, 28 bp, Artificial sequence
  Primer #2 for amplification of crt gene cluster
gagaaattatgttgtggatttggaatgc

SEQ ID NO: 21 - DNA, 61 bp, Artificial sequence
  Primer 5′kan(dxs)
tggaagcgctagcggactacatcatccagcgtaataaataacgtcttgagcgattgtgtag

SEQ ID NO: 22 - DNA, 65 bp, Artificial sequence
  Primer 5′kan(idi)
tctgatgcgcaagctgaagaaaaatgagcatggagaataatatgacgtcttgagcgattgtgtag

SEQ ID NO: 23 - DNA, 65 bp, Artificial sequence
  Primer 5′kan(ygbBP)
gacgcgtcgaagcgcgcacagtctgcggggcaaaacaatcgataacgtcttgagcgattgtgtag

SEQ ID NO: 24 - DNA, 60 bp, Artificial sequence
  Primer 5′kan(ispAdxs)
accatgacggggcgaaaaatattgagagtcagacattcatgtgtaggctggagctgcttc

SEQ ID NO: 25 - DNA, 64 bp, Artificial sequence
  Primer 3′kan
gaagacgaaagggcctcgtgatacgcctatttttataggttatatgaatatcctccttagttcc

SEQ ID NO: 26 - DNA, 50 bp, Artificial sequence
  Primer 5′-T5
ctaaggaggatattcatataacctataaaaataggcgtatcacgaggccc

SEQ ID NO: 27 - DNA, 70 bp, Artificial sequence
  Primer 3′-T5(dxs)
ggagtcgaccagtgccagggtcgggtatttggcaatatcaaaactcatagttaatttctcctctttaatg

SEQ ID NO: 28 - DNA, 68 bp, Artificial sequence
  Primer 3′-T5(idi)
tgggaactccctgtgcattcaataaaatgacgtgttccgtttgcatagttaatttctcctctttaatg

SEQ ID NO: 29 - DNA, 68 bp, Artificial sequence
  Primer 3′-T5(ygbBP)
cggccgccggaaccacggcgcaaacatccaaatgagtggttgccatagttaatttctcctctttaatg

SEQ ID NO: 30 - DNA, 62 bp, Artificial sequence
  Primer 3′-T5(ispAdxs)
cctgcttaacgcaggcttcgagttgctgcggaaagtccatagttaatttctcctctttaatg

SEQ ID NO: 31 - DNA, 65 bp, Artificial sequence
  Primer 5′-kanT5(ispB)
accataaaccctaagttgcctttgttcacagtaaggtaatcggggcgtcttgagcgattgtgtag

SEQ ID NO: 32 - DNA, 67 bp, Artificial sequence
  Primer 3′-kanT5(ispB)
cgccatatcttgcgcggttaactcattgattttttctaaattcatagttaatttctcctctttaatg

SEQ ID NO: 33 - DNA, 156 bp, Artificial sequence
  Phage T5 promoter sequence
ctataaaaataggcgtatcacgaggccctttcgtcttcacctcgagaaatcataaaaaatttatttgctttgtgagcggataacaattataatagattcaattgtgagcggataacaatttcacacagaattcattaaagaggagaaattaactca

SEQ ID NO: 34 - DNA, 65 bp, Artificial sequence
  Primer 5′-kanT5(dxs16a)
cactaacgcccgcacattgctgcgggctttttgattcatttcgcacgtcttgagcgattgtgtag

SEQ ID NO: 35 - DNA, 65 bp, Artificial sequence
  Primer 5′-kanT5(dxr16a)
taaagggctaagagtagtgtgctcttagcccttaattacgtttcccgtcttgagcgattgtgtag

SEQ ID NO: 36 - DNA, 65 bp, Artificial sequence
  Primer 5′-kanT5(lytB16a)
ctacaactggcgagatgcatagcgagtataatttgtattttgcgtcgtcttgagcgattgtgtag

SEQ ID NO: 37 - DNA, 51 bp, Artificial sequence
  Primer 3′-kanT5(dxs16a)
agtagagggaagtctttggaaagagccatagttaatttctcctctttaatg

SEQ ID NO: 38 - DNA, 51 bp, Artificial sequence
  Primer 3′-kanT5(dxr16a)
acggtgccgccgcaatgatgctgtccaccagttaatttctcctctttaatg

SEQ ID NO: 39 - DNA, 51 bp, Artificial sequence
  Primer 3′-kanT5(lytB16a)
ccacgggggtttgcgagtacgatttgcatagttaatttctcctctttaatg

SEQ ID NO: 40 - DNA, 55 bp, Artificial sequence
  Primer 5′-(dxs16a)
acagaattcattaaagaggagaaattaactatggctctttccaaagacttccctc

SEQ ID NO: 41 - DNA, 55 bp, Artificial sequence
  Primer 5′-(dxr16a)
acagaattcattaaagaggagaaattaactggtggacagcatcattgcggcggca

SEQ ID NO: 42 - DNA, 55 bp, Artificial sequence
  Primer 5′-(lytB16a)
acagaattcattaaagaggagaaattaactatgcaaatcgtactcgcaaaccccc

SEQ ID NO: 43 - DNA, 68 bp, Artificial sequence
  Primer 3′-(dxs16a)
aggagcgaagtgattatcagtatgctgttcatatagcctcgaattatcaagcgcaaaactgttcgatg

SEQ ID NO: 44 - DNA, 67 bp, Artificial sequence
  Primer 3′-(dxr16a)
ggcattttcactctggcaatgcgcataaacgctttcaaagtcctgttaagctaccaaggtcttgatg

SEQ ID NO: 45 - DNA, 68 bp, Artificial sequence
  Primer 3′-(lytB16a)
agtggcggacgggcaaacaagggtaacataggatcaatgagggttattgatcacgcttgcatatgttt

SEQ ID NO: 46 - DNA, 25 bp, Artificial sequence
  Primer T-kan
accggatatcaccacttatctgctc

SEQ ID NO: 47 - DNA, 25 bp, Artificial sequence
  Primer B-ispA
cctaataatgcgccatactgcatgg

SEQ ID NO: 48 - DNA, 32 bp, Artificial sequence
  Primer T-T5
taacctataaaaataggcgtatcacgaggccc

SEQ ID NO: 49 - DNA, 25 bp, Artificial sequence
  Primer B-idi
tcatgctgacctggtgaaggaatcc

SEQ ID NO: 50 - DNA, 25 bp, Artificial sequence
  Primer B-dxs(16a)
gcgatattgtatgtctgattcagga

SEQ ID NO: 51 - DNA, 25 bp, Artificial sequence
  Primer B-lytB(16a)
tccactggatgcgggaagctggcag

SEQ ID NO: 52 - DNA, 26 bp, Artificial sequence
  Primer B-dxs
tggcaacagtcgtagctcctgggtgg

SEQ ID NO: 53 - DNA, 25 bp, Artificial sequence
  Primer B-ygb
ccagcagcgcatgcaccgagtgttc

SEQ ID NO: 54 - DNA, 21 bp, Artificial sequence
  Primer Tn5PCRF
gctgagttgaaggatcagatc

SEQ ID NO: 55 - DNA, 21 bp, Artificial sequence
  Primer Tn5PCRR
cgagcaagacgtttcccgttg

SEQ ID NO: 56 - DNA, 25 bp, Artificial sequence
  Primer Kan-2 FP-1
acctacaacaaagctctcatcaacc

SEQ ID NO: 57 - DNA, 25 bp, Artificial sequence
  Primer Kan-2 RP-1
gcaatgtaacatcagagattttgag

SEQ ID NO: 58 - DNA, 20 bp, Artificial sequence
  Primer Y15_F
ggatcgatcttgagatgacc

SEQ ID NO: 59 - DNA, 24 bp, Artificial sequence
  Primer Y15_R
gctttcgtaattttcgcatttctg

SEQ ID NO: 60 - DNA, 25 bp, Artificial sequence
  Primer T-Tn5yjeR
gcaatgtaacatcagagattttgag

SEQ ID NO: 61 - DNA, 24 bp, Artificial sequence
  Primer B-yjeR
gctttcgtaattttcgcatttctg

SEQ ID NO: 62 - DNA, 26 bp, Artificial sequence
  Primer B-ispB
agtacagcaatcatcggacgaatacg

SEQ ID NO: 63 - DNA, 1845 bp, Artificial sequence
  Sequence yjeR::Tn5 mutant gene (transposon disrupted yjeR)
atgggcaaaacatctatgatacacgcaattgtggatcaatatagtcactgtgaatgggtggaaaatagcatgagtgccaatgaaaacaacctgatttggatcgatcttgagatgaccggtctggatcccgagcgcgatcgcattattgagattgccacgctggtgaccgatgccaacctgaatattctggcagaagggccgaccattgcagtacaccagtctgatgaacagctggcgctgatggatgactggaacgtgcgcacccataccgccagcgggctggtagagcgcgtgaaagcgagcacgatgggcgatcgggaagctgaactggcaacgctcgaatttttaaaacagtgggtgcctgcgggaaaatcgccgatttgcggtaacagcatcggtcaggaccgtcgtttcctgtttaaatacatgccggagctggaagcctacttccactaccgttatctcgatgtcagcaccctgaaagagctggcgcgccgctggaagccggaaattctggatggttttaccaagcaggggacgcatcaggcgatggatgatatccgtgaatcggtggcggagctggcttactacctgtctcttatacacatctcaaccctgaagcttgcatgcctgcaggtcgactctagaggatccccgccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaattcatcgatgatggttgagatgtgtataagagacaggcttactaccgcgagcattttatcaagctgtaa

SEQ ID NO: 64 - DNA, 8609 bp, Artificial sequence
  Plasmid pPCB15
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatatacactccgctagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccaaacgaattcgcccttttgacggtctgcgcaaaaaaacacgttcaccttactggcatttcggctgagcagttgctggctgatatcgatagccgccttgatcagttactgccggttcagggtgagcgggattgtgtgggtgccgcgatgcgtgaaggcacgctggcaccgggcaaacgtattcgtccgatgctgctgttattaacagcgcgcgatcttggctgtgcgatcagtcacgggggattactggatttagcctgcgcggttgaaatggtgcatgctgcctcgctgattctggatgatatgccctgcatggacgatgcgcagatgcgtcgggggcgtcccaccattcacacgcagtacggtgaacatgtggcgattctggcggcggtcgctttactcagcaaagcgtttggggtgattgccgaggctgaaggtctgacgccgatagccaaaactcgcgcggtgtcggagctgtccactgcgattggcatgcagggtctggttcagggccagtttaaggacctctcggaaggcgataaaccccgcagcgccgatgccatactgctaaccaatcagtttaaaaccagcacgctgttttgcgcgtcaacgcaaatggcgtccattgcggccaacgcgtcctgcgaagcgcgtgagaacctgcatcgtttctcgctcgatctcggccaggcctttcagttgcttgacgatcttaccgatggcatgaccgataccggcaaagacatcaatcaggatgcaggtaaatcaacgctggtcaatttattaggctcaggcgcggtcgaagaacgcctgcgacagcatttgcgcctggccagtgaacacctttccgcggcatgccaaaacggccattccaccacccaactttttattcaggcctggtttgacaaaaaactcgctgccgtcagttaaggatgctgcatgagccattttgcggtgatcgcaccgccctttttcagccatgttcgcgctctgcaaaaccttgctcaggaattagtggcccgcggtcatcgtgttacgttttttcagcaacatgactgcaaagcgctggtaacgggcagcgatatcggattccagaccgtcggactgcaaacgcatcctcccggttccttatcgcacctgctgcacctggccgcgcacccactcggaccctcgatgttacgactgatcaatgaaatggcacgtaccagcgatatgctttgccgggaactgcccgccgcttttcatgcgttgcagatagagggcgtgatcgttgatcaaatggagccggcaggtgcagtagtcgcagaagcgtcaggtctgccgtttgtttcggtggcctgcgcgctgccgctcaaccgcgaaccgggtttgcctctggcggtgatgcctttcgagtacggcaccagcgatgcggctcgggaacgctataccaccagcgaaaaaatttatgactggctgatgcgacgtcacgatcgtgtgatcgcgcatcatgcatgcagaatgggtttagccccgcgtgaaaaactgcatcattgtttttctccactggcacaaatcagccagttgatccccgaactggattttccccgcaaagcgctgccagactgctttcatgcggttggaccgttacggcaaccccaggggacgccggggtcatcaacttcttattttccgtccccggacaaaccccgtatttttgcctcgctgggcaccctgcagggacatcgttatggcctgttcaggaccatcgccaaagcctgcgaagaggtggatgcgcagttactgttggcacactgtggcggcctctcagccacgcaggcaggtgaactggcccggggcggggacattcaggttgtggattttgccgatcaatccgcagcactttcacaggcacagttgacaatcacacatggtgggatgaatacggtactggacgctattgcttcccgcacaccgctactggcgctgccgctggcatttgatcaacctggcgtggcatcacgaattgtttatcatggcatcggcaagcgtgcgtctcggtttactaccagccatgcgctggcgcggcagattcgatcgctgctgactaacaccgattacccgcagcgtatgacaaaaattcaggccgcattgcgtctggcaggcggcacaccagccgccgccgatattgttgaacaggcgatgcggacctgtcagccagtactcagtgggcaggattatgcaaccgcactatgatctcattctggtcggtgccggtctggctaatggccttatcgcgctccggcttcagcaacagcatccggatatgcggatcttgcttattgaggcgggtcctgaggcgggagggaaccatacctggtcctttcacgaagaggatttaacgctgaatcagcatcgctggatagcgccgcttgtggtccatcactggcccgactaccaggttcgtttcccccaacgccgtcgccatgtgaacagtggctactactgcgtgacctcccggcatttcgccgggatactccggcaacagtttggacaacatttatggctgcataccgcggtttcagccgttcatgctgaatcggtccagttagcggatggccggattattcatgccagtacagtgatcgacggacggggttacacgcctgattctgcactacgcgtaggattccaggcatttatcggtcaggagtggcaactgagcgcgccgcatggtttatcgtcaccgattatcatggatgcgacggtcgatcagcaaaatggctaccgctttgtttataccctgccgctttccgcaaccgcactgctgatcgaagacacacactacattgacaaggctaatcttcaggccgaacgggcgcgtcagaacattcgcgattatgctgcgcgacagggttggccgttacagacgttgctgcgggaagaacagggtgcattgcccattacgttaacgggcgataatcgtcagttttggcaacagcaaccgcaagcctgtagcggattacgcgccgggctgtttcatccgacaaccggctactccctaccgctcgcggtggcgctggccgatcgtctcagcgcgctggatgtgtttacctcttcctctgttcaccagacgattgctcactttgcccagcaacgttggcagcaacaggggtttttccgcatgctgaatcgcatgttgtttttagccggaccggccgagtcacgctggcgtgtgatgcagcgtttctatggcttacccgaggatttgattgcccgcttttatgcgggaaaactcaccgtgaccgatcggctacgcattctgagcggcaagccgcccgttcccgttttcgcggcattgcaggcaattatgacgactcatcgttgaagagcgactacatgaaaccaactacggtaattggtgcgggctttggtggcctggcactggcaattcgtttacaggccgcaggtattcctgttttgctgcttgagcagcgcgacaagccgggtggccgggcttatgtttatcaggagcagggctttacttttgatgcaggccctaccgttatcaccgatcccagcgcgattgaagaactgtttgctctggccggtaaacagcttaaggattacgtcgagctgttgccggtcacgccgttttatcgcctgtgctgggagtccggcaaggtcttcaattacgataacgaccaggcccagttagaagcgcagatacagcagtttaatccgcgcgatgttgcgggttatcgagcgttccttgactattcgcgtgccgtattcaatgagggctatctgaagctcggcactgtgccttttttatcgttcaaagacatgcttcgggccgcgccccagttggcaaagctgcaggcatggcgcagcgtttacagtaaagttgccggctacattgaggatgagcatcttcggcaggcgttttcttttcactcgctcttagtgggggggaatccgtttgcaacctcgtccatttatacgctgattcacgcgttagaacgggaatggggcgtctggtttccacgcggtggaaccggtgcgctggtcaatggcatgatcaagctgtttcaggatctgggcggcgaagtcgtgcttaacgcccgggtcagtcatatggaaaccgttggggacaagattcaggccgtgcagttggaagacggcagacggtttgaaacctgcgcggtggcgtcgaacgctgatgttgtacatacctatcgcgatctgctgtctcagcatcccgcagccgctaagcaggcgaaaaaactgcaatccaagcgtatgagtaactcactgtttgtactctattttggtctcaaccatcatcacgatcaactcgcccatcataccgtctgttttgggccacgctaccgtgaactgattcacgaaatttttaaccatgatggtctggctgaggatttttcgctttatttacacgcaccttgtgtcacggatccgtcactggcaccggaagggtgcggcagctattatgtgctggcgcctgttccacacttaggcacggcgaacctcgactgggcggtagaaggaccccgactgcgcgatcgtatttttgactaccttgagcaacattacatgcctggcttgcgaagccagttggtgacgcaccgtatgtttacgccgttcgatttccgcgacgagctcaatgcctggcaaggttcggccttctcggttgaacctattctgacccagagcgcctggttccgaccacataaccgcgataagcacattgataatctttatctggttggcgcaggcacccatcctggcgcgggcattcccggcgtaatcggctcggcgaaggcgacggcaggcttaatgctggaggacctgatttgacgaatacgtcattactgaatcatgccgtcgaaaccatggcggttggctcgaaaagctttgcgactgcatcgacgcttttcgacgccaaaacccgtcgcagcgtgctgatgctttacgcatggtgccgccactgcgacgacgtcattgacgatcaaacactgggctttcatgccgaccagccctcttcgcagatgcctgagcagcgcctgcagcagcttgaaatgaaaacgcgtcaggcctacgccggttcgcaaatgcacgagcccgcttttgccgcgtttcaggaggtcgcgatggcgcatgatatcgctcccgcctacgcgttcgaccatctggaaggttttgccatggatgtgcgcgaaacgcgctacctgacactggacgatacgctgcgttattgctatcacgtcgccggtgttgtgggcctgatgatggcgcaaattatgggcgttcgcgataacgccacgctcgatcgcgcctgcgatctcgggctggctttccagttgaccaacattgcgcgtgatattgtcgacgatgctcaggtgggccgctgttatctgcctgaaagctggctggaagaggaaggactgacgaaagcgaattatgctgcgccagaaaaccggcaggccttaagccgtatcgccgggcgactggtacgggaagcggaaccctattacgtatcatcaatggccggtctggcacaattacccttacgctcggcctgggccatcgcgacagcgaagcaggtgtaccgtaaaattggcgtgaaagttgaacaggccggtaagcaggcctgggatcatcgccagtccacgtccaccgccgaaaaattaacgcttttgctgacggcatccggtcaggcagttacttcccggatgaagacgtatccaccccgtcctgctcatctctggcagcgcccgatctagccgcatgcctttctctcagcgtcgcctgaagtttagataacggtggcgcgtacagaaaaccaaaggacacgcagccctcttttccccttacagcatgatgcatacggtgggccatgtataaccgtttcaggtagcctttgcgcggtatgtagcggaacggccagcgctggtgtaccagtccgtcgtggaccataaaatacagtaaaccataagcggtcatgcctgcaccaatccactggagcggccagattcctgtactgccgaagtaaatcagggcaatcgacacaatggcgaataccacggcatagagatcgttaacttcaaatgcgcctttacgcggttcatgatgtgaaagatgccagccccaaccccagccgtgcatgatgtatttatgtgccagtgcagcaaccacttccatgccgaccacggtgacaaacacgatcagggcattccaaatccacaacataatttctcaagggcgaattcgcggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattt

SEQ ID NO: 65 - DNA, 6329 bp, Artificial sequence
  Plasmid pKD46
catcgatttattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctctaaggaggttataaaaaatggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgaatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatgacgcatcctcacgataatatccgggtaggcgcaatcactttcgtctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaatccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 66 - DNA, 3423 bp, Artificial sequence
  Plasmid pSUH5
agattgcagcattacacgtcttgagcgattgtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcactataaaaataggcgtatcacgaggccctttcgtcttcacctcgagaaatcataaaaaatttatttgctttgtgagcggataacaattataatagattcaattgtgagcggataacaatttcacacagaattcattaaagaggagaaattaactcatatggaccatggctaattcccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgcggatcttgcggccgcaaaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgcatcgatggccccccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgc